(19) United States
(12) Patent Application Publication
(10) Pub. No.: US 2022/0322709 A1
(43) Pub. Date: Oct. 13, 2022

(54) METHODS AND COMPOSITIONS FOR IMPROVED TASTE QUALITY

(71) Applicant: ALMENDRA PTE. LTD., Singapore (SG)

(72) Inventors: Grant E. DUBOIS, Peoria, AZ (US); William Robert HASTINGS, Lithia, FL (US)

(21) Appl. No.: 17/709,866

(22) Filed: Mar. 31, 2022

Related U.S. Application Data
(60) Provisional application No. 63/169,175, filed on Mar. 31, 2021.

(51) Int. Cl. A23L 2/60; A61K 9/00; A61K 47/12; A61K 47/02

(52) U.S. Cl. CPC A23L 2/60; A61K 9/0095; A61K 9/0056; A61K 47/12; A61K 47/02

(57) ABSTRACT

In various aspects, the present disclosure relates to food products, beverage products, and pharmaceutical or medicinal products comprising a taste modulator component. The taste modulator component improves key properties, including bitter off-taste and mouthfeel characteristics. In further aspects, the present disclosure pertains to methods of modulating bitter taste in a food product, beverage product, or medicament. This abstract is intended as a scanning tool for purposes of searching in the particular art and is not intended to be limiting of the present disclosure.

### CROSS-REFERENCE TO RELATED APPLICATIONS

This Application claims the benefit of U.S. Provisional Application No. 63/169,175, filed on Mar. 31, 2021, which is incorporated herein by reference in its entirety.

### FIELD OF INVENTION

The present disclosure relates to taste modulator compositions comprising one or more taste modulator components as disclosed herein. The taste modulator component improves key properties associated with edible liquids and foodstuffs, including reduction or elimination of bitter off taste.

### BACKGROUND

The taste system provides sensory information about the chemical composition of the external world. Taste transduction is one of the most sophisticated forms of chemical-triggered sensation in animals, and is found throughout the animal kingdom, from simple metazoans to the most complex of vertebrates. Mammals are believed to have five basic taste modalities: sweet, bitter, sour, salty, and umami (the taste of monosodium glutamate).

Many food and beverage products are associated with undesirable bitterness due to either intrinsic characteristics of various components—natural or added—in the product, including sweeteners, processing aids, and the like. Bitterness in terms of both the intensity of the bitterness and the time of linger in the mouth are strongly unappealing characteristics for many consumers and can limit consumer acceptance of such products.

Moreover, many pharmaceutical formulations and medicinal products, e.g., liquid or buccal formulations, are associated with bitter taste properties due to the nature of the active ingredient and/or various excipients, fillers, and the like. A bitter pharmaceutical product can result in poor patient compliance with using such a pharmaceutical product, thereby leading to poorer clinical outcomes.

Despite advances in compositions and methods for foods, beverages, medicaments and other products, there is a scarcity of taste modulator compositions with suitably low cost for widespread use and likelihood for consumer acceptance. These needs and other needs are satisfied by the present disclosure.

### SUMMARY

In accordance with the purpose(s) of the present disclosure, as embodied and broadly described herein, the disclosure, in one aspect, relates to food products, beverage products, and pharmaceutical or medicinal products comprising a taste modulator component. The taste modulator component improves key properties, including bitter off-taste and mouthfeel characteristics. In further aspects, the disclosed taste modulator composition can further comprise an additional Calcium Sensing Receptor (CaSR) modulator.

Disclosed herein are products comprising: a taste modulator composition comprising of a first taste modulator component comprising a first salt having a first cation Mg²⁺ and a first anion; a second taste modulator component consisting essentially of a second salt having a second cation Ca²⁺ and a second anion; and optionally a third taste modulator component consisting essentially of a third salt having a third cation selected from K⁺ and Na⁺ and a third anion; wherein the first taste modulator component is at a concentration of from about 0.1 mM to about 10 mM; wherein the second taste modulator component is at a concentration of from about 0.1 mM to about 10 mM; wherein the third taste modulator component, when present, is at a concentration of from about 0.1 mM to about 25 mM; wherein the product is a food product, a beverage product, or a medicament; wherein the product is associated with a bitter taste in the absence of the taste modulator composition; wherein the product comprising the taste modulator composition has a bitterness that is lower compared to a baseline product; wherein the baseline product consists essentially of the same components as the product without the taste modulator component; and wherein the bitterness is determined using a sensory panel study.

Also disclosed herein are methods for inhibiting bitterness in a product, the method comprising: adding a taste modulator composition to a product; wherein the taste modulator composition comprises a first taste modulator component consisting essentially of a first salt having a first cation Mg²⁺ and a first anion; a second taste modulator component consisting essentially of a second salt having a second cation Ca²⁺ and a second anion; and optionally a third taste modulator component consisting essentially of a third salt having a third cation selected from K⁺ and Na⁺ and a third anion; wherein the first taste modulator component is at a concentration of from about 0.1 mM to about 10 mM; wherein the second taste modulator component is at a concentration of from about 0.1 mM to about 10 mM; wherein the third taste modulator component, when present, is at a concentration of from about 0.1 mM to about 25 mM; wherein the product is a food product, a beverage product, or a medicament; wherein the product is associated with a bitter taste in the absence of adding the taste modulator composition; wherein the product comprising the taste modulator composition has a bitterness that is lower compared to a baseline product; wherein the baseline product consists essentially of the same components as the product without the taste modulator component; and wherein the bitterness is determined using a sensory panel study.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described aspects are usable in all aspects of the disclosure taught herein. Furthermore, the individual features of the dependent claims, as well as all optional and preferred features and modifications of the described aspects are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows chemical structures for representative Stevia-derived compounds as indicated (Rebaudioside A, Rebaudioside B, and Rebaudioside C).

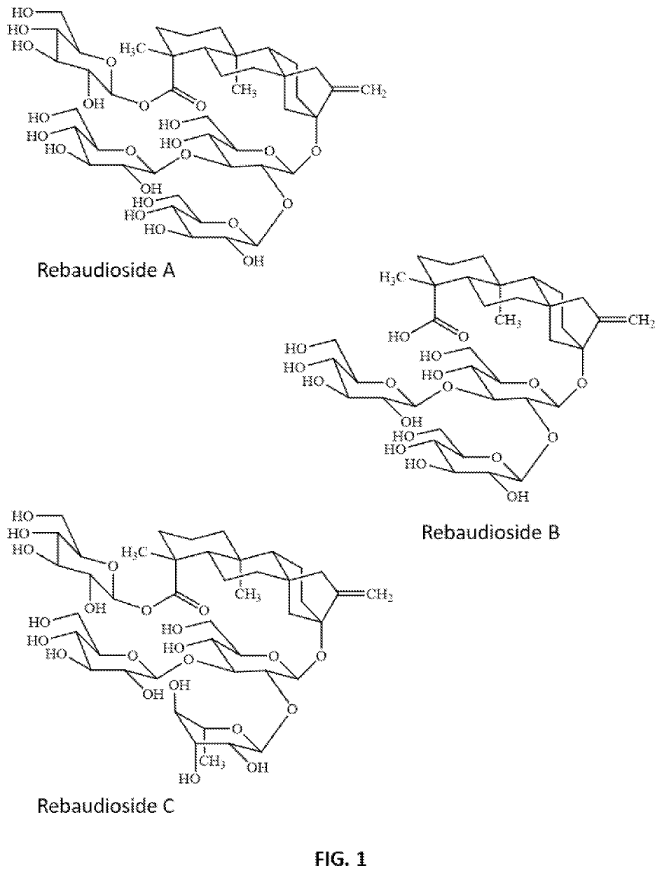

FIG. 2 shows chemical structures for representative Stevia-derived compounds as indicated (Rebaudioside D, Rebaudioside E, and Rebaudioside F).

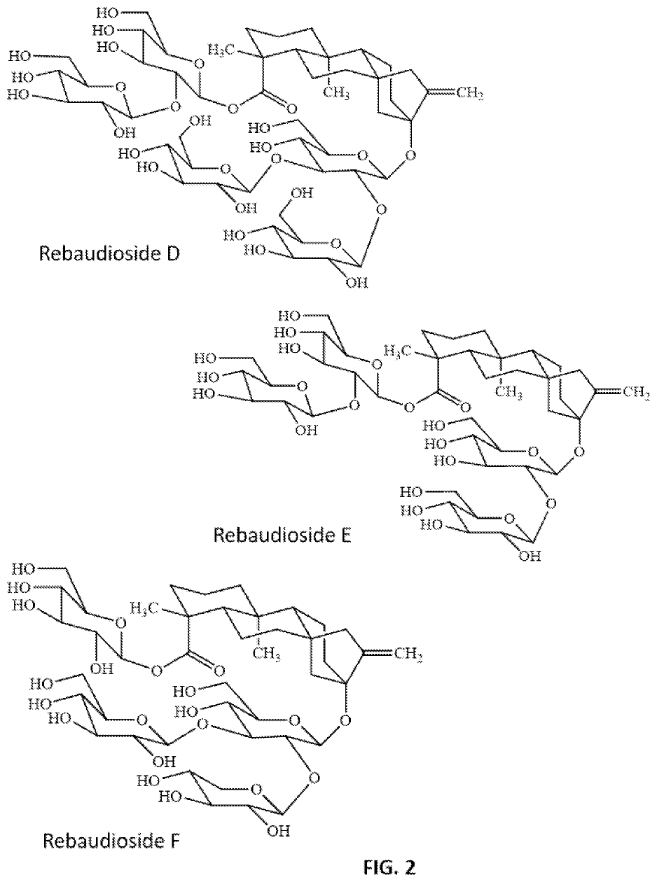

FIG. 3 shows chemical structures for representative Stevia-derived compounds as indicated (Rebaudioside M and Rebaudioside N).

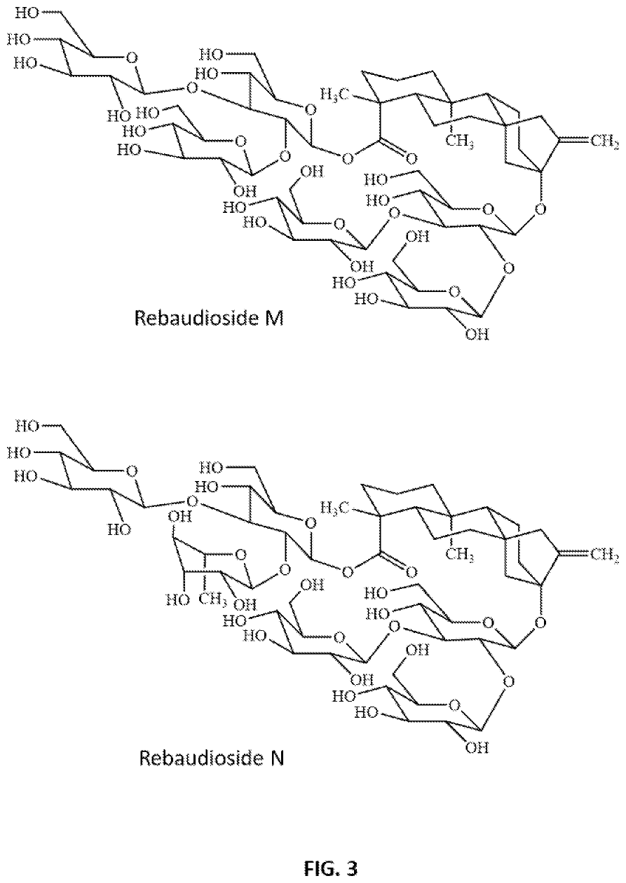

FIG. 4 shows chemical structures for representative Stevia-derived compounds as indicated (Steviol, Steviolmonoside, Steviolbioside, and Stevioside).

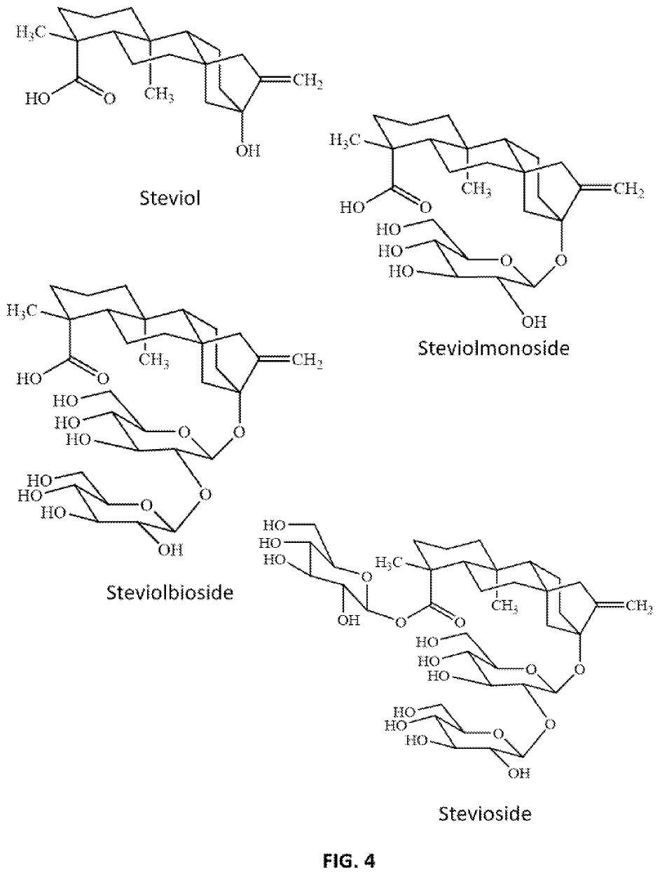

FIG. 5 shows chemical structures for representative Stevia-derived compounds as indicated (Rubusoside and Dulcoside A).

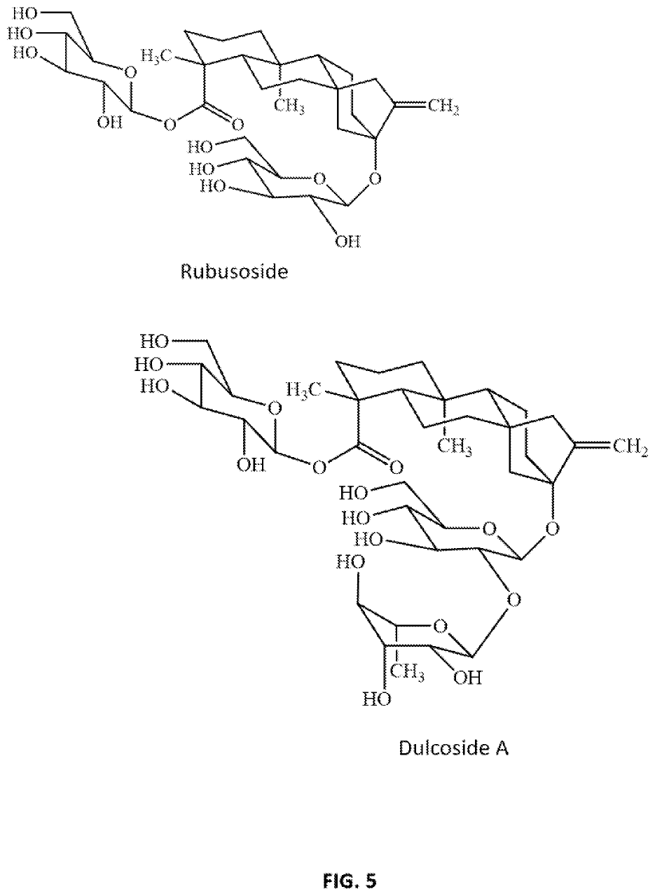

FIG. 6 shows representative data for the effect of a disclosed taste modulator composition on sensory qualities, e.g., bitterness, with a tabletop Stevia sweetener preparation.

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

Many modifications and other aspects disclosed herein will come to mind to one skilled in the art to which the disclosed compositions and methods pertain having the benefit of the teachings presented in the foregoing descriptions and the associated drawings. Therefore, it is to be understood that the disclosure is not to be limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual aspects described and illustrated herein has discrete components and features which may be readily separated from or combined with the features of any of the other several aspects without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosure. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Additionally, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a,” “an” and “the” include plural referents unless the context clearly dictates otherwise. Thus, for example, reference to “flavorant or flavor,” “a sweetening agent,” “a cation,” or “a taste modulator,” including, but not limited to, two or more such flavorant or flavors, sweetening agents, cations, or taste modulators, including combinations of sweetening agents, cations, and taste modulators, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, “Stevia sweetener,” “Stevia-derived sweetener,” and “Stevia rebaudiana-derived sweetener” can be used interchangeably. It is understood that a Stevia sweetener can refer to an extract, concentrate, juice, or other preparation obtained from leaves and/or other plant structures (e.g., fruits, seeds, stems or fleshy plant parts) of a plant in the genus Stevia, in some cases from the Stevia rebaudiana plant; or a mixture of one or more purified or partially purified component or compound from a plant in the genus Stevia, in some cases from the Stevia rebaudiana plant, such as steviol glycosides, stevioside, rebaudioside A, rebaudioside B, rebaudioside C, rebaudioside F, rebaudioside F, dulcoside A, steviolbioside, rubusoside, as well as other steviol glycosides found in a plant in the genus Stevia, in some cases from the Stevia rebaudiana plant, and mixtures thereof; glucosylated steviol glucosides; and combinations, mixtures, and kits comprising.

As used herein, the term “steviol glycoside(s)” refers to glycosides of steviol, including, but not limited to, naturally occurring steviol glycosides, e.g. Rebaudioside A, Rebaudioside B, Rebaudioside C, Rebaudioside D, Rebaudioside E, Rebaudioside F, Rebaudioside G, Rebaudioside H, Rebaudioside I, Rebaudioside J, Rebaudioside K, Rebaudioside L, Rebaudioside M (also referred to as Rebaudioside X), Rebaudioside N, Rebaudioside O, Stevioside, Steviolbioside, Dulcoside A, Rubusoside, etc., or synthetic steviol glycosides, e.g. enzymatically glucosylated steviol glycosides and combinations thereof.

As used herein, “monk fruit sweetener,” “monk fruit-derived sweetener,” “luo han guo sweetener,” luo han guo-derived sweetener,” and “Siraitia grosvenorii-derived sweetener” can be used interchangeably. It is understood that a monk fruit sweetener can refer to an extract, concentrate, juice, or other preparation obtained from leaves and/or other plant structures (e.g., fruits, seeds, stems or fleshy plant parts) of a plant in the genus Siraitia, in some cases from the Siraitia grosvenorii plant; or a mixture of one or more purified or partially purified component or compound from a plant in the genus Siraitia, in some cases from the Siraitia grosvenorii plant, such as mogroside I, mogroside II, mogroside Ill, mogroside IV (esgoside), neomogroside, 11-oxo-mogroside V, mogroside VI, mogroside V, and siamenoside I, as well as other mogrosides and triterpene glycosides found in a plant in the genus Siraitia, in some cases from the Siraitia grosvenorii plant, and mixtures thereof; glucosylated mogrosides; and combinations, mixtures, and kits comprising any of the foregoing.

As used herein, the terms “high potency sweetener,” “high-potency sweetener,” and “non-nutritive sweetener,” are terms that can be used interchangeably, refers to a sweetening agent that can be synthetic or natural in origin, with a sweetness potency greater than sucrose, e.g., a sweetness potency that can be about 2-fold-15,000-fold greater than sucrose. Such sweeteners are essentially non-caloric and used widely in manufacturing of diet and reduced calorie food. In general, non-nutritive sweeteners do not affect the blood glucose level and provide little or no nutritive value. Non-limiting examples of synthetic non-nutritive sweeteners include sucralose, potassium acesulfame, aspartame, alitame, saccharin, neohesperidin dihydrochalcone and synthetic analogues, cyclamate, neotame, dulcin, suosan, N—[N-[3-(3-hydroxy-4-methoxyphenyl)propyl]-L-α-aspartyl]-L-phenylalanine 1-methyl ester, N—[N-[3-(3-hydroxy-4-methoxyphenyl)-3-methylbutyl]-L-α-aspartyl]-L-phenylalanine 1-methyl ester, N—[N-[3-(3-methoxy-4-hydroxyphenyl)propyl]-L-α-aspartyl]-L-phenylalanine 1-methyl ester, salts thereof, and the like. Additional examples of synthetic non-nutritive sweeteners are described herein below. Non-limiting examples of natural non-nutritive sweeteners include Stevioside, Rebaudioside A, Rebaudioside B, Rebaudioside C, Rebaudioside D, Rebaudioside E, Rebaudioside F, Rebaudioside M, Steviolbioside, Dulcoside A, Rubusoside, mogrosides, brazzein, neohesperidin dihydrochalcone (NHDC), glycyrrhizic acid and its salts, thaumatin, perillartine, hernandulcin, mukuroziosides, baiyunoside, phlomisoside-1, dimethyl-hexahydrofluorene-dicarboxylic acid, abrusosides, periandrin, carnosiflosides, cyclocarioside, pterocaryosides, polypodoside A, brazzein, hernandulcin, phillodulcin, glycyphyllin, phlorizin, trilobatin, dihydroflavonol, dihydroquercetin-3-acetate, neoastilibin, trans-cinnamaldehyde, monatin and its salts, selligueain A, hematoxylin, monellin, osladin, pterocaryoside A, pterocaryoside B, mabinlin, pentadin, miraculin, curculin, neoculin, chlorogenic acid, cynarin, siamenoside and others. Additional examples of natural non-nutritive sweeteners are described herein below. It should be noted that non-nutritive sweeteners can be derived from the modification of natural non-nutritive sweeteners, for example, by fermentation, enzymatic treatment, or derivatization.

A “flavor” herein refers to the perception of taste and/or smell in a subject, which include sweet, sour, salty, bitter, umami, and others. The subject may be a human or an animal.

A “flavoring agent” herein refers to a compound or a biologically acceptable salt thereof that induces a flavor or taste in an animal or a human.

A “flavor modifier” herein refers to a compound or biologically acceptable salt thereof that modulates, including enhancing or potentiating, and inducing, the tastes and/or smell of a natural or synthetic flavoring agent in an animal or a human.

A “flavor enhancer” herein refers to a compound or biologically acceptable salt thereof that enhances and/or multiplies the tastes or smell of a natural or synthetic flavoring agent, or a comestible composition comprising the flavor enhancer.

As used herein, “flavors with modifying properties” or “FMP” can be used interchangeably, and refer to those generally recognized as safe (GRAS) ingredients that enhance, subdue or otherwise affect other flavors without themselves being sweeteners or flavorings. The Flavor and Extracts Manufacturing Association (FEMA) has developed a protocol published in the November 2013 issue of Food Technology.

As used herein, the term “FEMA GRAS” means that an ingredient has been designated as generally recognized as safe by an independent Flavor Expert Panel for use in flavors, e.g., see Expert Panel, Toxicology, Decision Tree, Consumption Ratio, and Chart 486—FEMA GRAS Lists Numbers Included, FDA GRAS, Bulk Flavor Labeling Statement.

As used herein, “taste” refers to a sensation caused by activation of target taste receptor cells in the taste buds of a subject. Taste can be selected from the group consisting of sweet, sour, salt, bitter, and umami. A taste can be elicited in a subject by a “tastant,” which can be a synthetic tastant, a tastant prepared from a natural source (i.e., a natural tastant), or combinations thereof.

As used herein, the terms “modulates” or “modifies” refers to an increase or decrease in the amount, quality or effect of a particular activity of a receptor and/or an increase or decrease in the expression, activity or function of a receptor. “Modulators,” as used herein, refer to any inhibitory or activating compounds identified using in silico, in vitro and/or in vivo assays for, e.g., agonists, antagonists and their homologs, including fragments, variants and mimetics.

“Inducers,” “activators” or “agonists,” as used herein, refer to modulating compounds that increase, induce, stimulate, open, activate, facilitate, enhance activation, sensitize or upregulate a receptor or pathway of interest.

The terms “polypeptide,” “peptide,” “amino acid sequence” and “protein,” used interchangeably herein, refer to a molecule formed from the linking of at least two amino acids.

The link between one amino acid residue and the next is an amide bond and is sometimes referred to as a peptide bond. The terms can apply to amino acid polymers in which one or more amino acid residue is an artificial chemical mimetic of a corresponding naturally occurring amino acid, as well as to naturally occurring amino acid polymers and non-naturally occurring amino acid polymers.

The term “amino acid,” as used herein, refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate and O-phosphoserine. Amino acid analogs and derivatives can refer to compounds that have the same basic chemical structure as a naturally occurring amino acid, i.e., a carbon that is bound to a hydrogen, a carboxyl group, an amino group and an R group, e.g., homoserine, norleucine, methionine sulfoxide and methionine sulfone. Such analogs can have modified R groups (e.g., norleucine) or modified peptide backbones, but retain the same basic chemical structure as a naturally occurring amino acid. Amino acid mimetics means chemical compounds that have a structure that is different from the general chemical structure of an amino acid, but that functions in a manner similar to a naturally occurring amino acid.

As used herein, the term “effective amount” refers to an amount that is sufficient to achieve the desired modification of a physical property of the composition or material. For example, an “effective amount” of a disclosed taste modulator composition or a sweetening agent refers to an amount that is sufficient to achieve the desired improvement in the property modulated by the formulation component, e.g. achieving the desired level of sweetness, sweetness appearance time, sweetness linger, sweetness desensitization, body/mouthfeel, sourness, saltiness, bitterness, or astringency. The specific level in terms of wt % in a composition required as an effective amount will depend upon a variety of factors including the amount and type of sweetener, amount and type of taste modulator, amount and type of salts and/or cations, and end use of the product made using the composition.

As used herein, the terms “optional” or “optionally” means that the subsequently described event or circumstance can or cannot occur, and that the description includes instances where said event or circumstance occurs and instances where it does not.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia.

For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as pain, cancer, and/or cardiovascular disease. The effect can be therapeutic in terms of a partial or complete cure of a disease, condition, symptom or adverse effect attributed to the disease, disorder, or condition. The term “treatment” as used herein can include any treatment of a clinical condition in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1-to-C 6 alkyl esters and C 5-to-C 7 cycloalkyl esters, although C 1-to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1-to-C 6 alkyl amines and secondary C 1-to-C 6 dialkyl amines. In the case of secondary amines, the amine can also be in the form of a 5- or 6-membered heterocycle containing one nitrogen atom. Amides derived from ammonia, C 1-to-C 3 alkyl primary amides and C 1-to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, a “medicament” comprises a pharmaceutical composition comprising a dosage of a therapeutic agent in a therapeutically effective amount (or prophylactically effective amount) for administration to a subject to treat a clinical condition.

Unless otherwise specified, temperatures referred to herein are based on atmospheric pressure (i.e. one atmosphere).

### Taste Modulator Compositions

In various aspects, the present disclosure, relates to taste modulator compositions comprising one or more taste modulator components. The taste modulator component improves key properties associated with edible liquids and foodstuffs, including overall taste response; mitigation of various flavor profile issues, e.g., bitter taste, including bitterness intensity and/or bitterness linger; improves desensitization/adaptation profile issues; and improves body/mouthfeel characteristics.

In various aspects, a composition comprising a disclosed taste modulator composition has one or more of the following characteristics modified as indicated when compared to the same composition without the disclosed taste modulator composition: bitter taste, including bitterness intensity and/or bitterness linger, sweetness intensity maxima enhancement, sweetness onset time acceleration, sweetness linger attenuation, sweetness desensitization attenuation, and/or mouthfeel enhancement. That is, a composition comprising a disclosed taste modulator composition has modified one of the foregoing characteristics as compared to essentially the same composition except with the same flavorant or flavor at the same concentration with the disclosed taste modulator composition omitted as determined in a sensory panel study as described herein.

In various aspects, one or more of these characteristics in a composition comprising a disclosed taste modulator composition, when determined in a sensory panel study as described herein, is improved by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%; or any range encompassed by the foregoing values; or any combination of the foregoing values. In various aspects, one or more of these characteristics in a composition comprising a disclosed taste modulator composition with a flavorant or flavor is improved, when determined in a sensory panel study as described herein, is improved by about 1.1-fold, about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 2.6-fold, about 2.7-fold, about 2.8-fold, about 2.9-fold, about 3-fold, about 3.1-fold, about 3.2-fold, about 3.3-fold, about 3.4-fold, about 3.5-fold, about 3.6-fold, about 3.7-fold, about 3.8-fold, about 3.9-fold, about 4-fold, about 4.1-fold, about 4.2-fold, about 4.3-fold, about 4.4-fold, about 4.5-fold, about 4.6-fold, about 4.7-fold, about 4.8-fold, about 4.9-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, about 20-fold, about 21-fold, about 22-fold, about 23-fold, about 24-fold, about 25-fold, about 26-fold, about 27-fold, about 28-fold, about 29-fold, about 30-fold, about 31-fold, about 32-fold, about 33-fold, about 34-fold, about 35-fold, about 36-fold, about 37-fold, about 38-fold, about 39-fold, about 40-fold, about 41-fold, about 42-fold, about 43-fold, about 44-fold, about 45-fold, about 46-fold, about 47-fold, about 48-fold, about 49-fold, about 50-fold, about 51-fold, about 52-fold, about 53-fold, about 54-fold, about 55-fold, about 56-fold, about 57-fold, about 58-fold, about 59-fold, about 60-fold, about 61-fold, about 62-fold, about 63-fold, about 64-fold, about 65-fold, about 66-fold, about 67-fold, about 68-fold, about 69-fold, about 70-fold, about 71-fold, about 72-fold, about 73-fold, about 74-fold, about 75-fold, about 76-fold, about 77-fold, about 78-fold, about 79-fold, about 80-fold, about 81-fold, about 82-fold, about 83-fold, about 84-fold, about 85-fold, about 86-fold, about 87-fold, about 88-fold, about 89-fold, about 90-fold, about 91-fold, about 92-fold, about 93-fold, about 94-fold, about 95-fold, about 96-fold, about 97-fold, about 98-fold, about 99-fold, about 100-fold; or any range encompassed by the foregoing values; or any combination of the foregoing values.

In various aspects, a composition comprising a disclosed taste modulator composition has enhanced mouthfeel compared to the same composition without the disclosed taste modulator composition. That is, a composition comprising a disclosed taste modulator composition has modified mouthfeel, specifically enhanced mouthfeel, as compared to essentially the same composition with the disclosed taste modulator composition omitted as determined in a sensory panel study as described herein.

In various aspects, a composition comprising a disclosed taste modulator composition has modulated bitter taste, including bitterness intensity and/or bitterness linger, compared to the same composition without the disclosed taste modulator composition. That is, a composition comprising a disclosed taste modulator composition has modified bitter taste, specifically modulated bitter taste, including bitterness intensity and/or bitterness linger, as compared to essentially the same composition with the disclosed taste modulator composition omitted as determined in a sensory panel study as described herein.

In various aspects, the bitter taste or bitterness profile in a composition comprising a disclosed taste modulator composition is modulated, e.g., decreased, diminished, or inhibited, when determined in a sensory panel study as described herein, is improved by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%; or any range encompassed by the foregoing values; or any combination of the foregoing values. In various aspects, one or more of these characteristics in a composition comprising a disclosed taste modulator composition is improved, including mitigation or diminution of bitter taste, when determined in a sensory panel study as described herein, is improved by about 1.1-fold, about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 2.6-fold, about 2.7-fold, about 2.8-fold, about 2.9-fold, about 3-fold, about 3.1-fold, about 3.2-fold, about 3.3-fold, about 3.4-fold, about 3.5-fold, about 3.6-fold, about 3.7-fold, about 3.8-fold, about 3.9-fold, about 4-fold, about 4.1-fold, about 4.2-fold, about 4.3-fold, about 4.4-fold, about 4.5-fold, about 4.6-fold, about 4.7-fold, about 4.8-fold, about 4.9-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, about 20-fold, about 21-fold, about 22-fold, about 23-fold, about 24-fold, about 25-fold, about 26-fold, about 27-fold, about 28-fold, about 29-fold, about 30-fold, about 31-fold, about 32-fold, about 33-fold, about 34-fold, about 35-fold, about 36-fold, about 37-fold, about 38-fold, about 39-fold, about 40-fold, about 41-fold, about 42-fold, about 43-fold, about 44-fold, about 45-fold, about 46-fold, about 47-fold, about 48-fold, about 49-fold, about 50-fold, about 51-fold, about 52-fold, about 53-fold, about 54-fold, about 55-fold, about 56-fold, about 57-fold, about 58-fold, about 59-fold, about 60-fold, about 61-fold, about 62-fold, about 63-fold, about 64-fold, about 65-fold, about 66-fold, about 67-fold, about 68-fold, about 69-fold, about 70-fold, about 71-fold, about 72-fold, about 73-fold, about 74-fold, about 75-fold, about 76-fold, about 77-fold, about 78-fold, about 79-fold, about 80-fold, about 81-fold, about 82-fold, about 83-fold, about 84-fold, about 85-fold, about 86-fold, about 87-fold, about 88-fold, about 89-fold, about 90-fold, about 91-fold, about 92-fold, about 93-fold, about 94-fold, about 95-fold, about 96-fold, about 97-fold, about 98-fold, about 99-fold, about 100-fold; or any range encompassed by the foregoing values; or any combination of the foregoing values. In the foregoing it is understood that the improvement in bitter taste or bitterness profile of a composition comprising a disclosed taste modulator composition compared to essentially the same composition except without the disclosed taste modulator composition when assessed in a sensory panel study as described herein.

In a further aspect, the disclosed taste modulator compositions comprise a first taste modulator component comprising a first salt having a first cation selected from Na⁺, K⁺, Ca²⁺, and Mg²⁺. The taste modulator compositions can optionally further comprise one or more additional taste modulator components, e.g., a second taste modulator component comprising a second salt having a second cation selected from Na⁺, K⁺, Ca²⁺, and Mg²⁺; a third taste modulator component comprising a third salt having a third cation selected from Na⁺, K⁺, Ca²⁺, and Mg²⁺; and a fourth taste modulator component comprising a fourth salt having a fourth cation selected from Na⁺, K⁺, Ca²⁺, and Mg²⁺. In some instances, the additional taste modulator components each comprise a different cation selected from Na⁺, K⁺, Ca²⁺, and Mg²⁺.

In various aspects, a disclosed taste modulator composition comprises a first salt having a first cation independently selected from Na⁺, K⁺, Ca²⁺, and Mg²⁺; optionally a second salt having a second cation independently selected from Na⁺, K⁺, Ca²⁺, and Mg²⁺; optionally a third salt having a third cation independently selected from Na⁺, K⁺, Ca²⁺, and Mg²⁺; and optionally a fourth salt having a fourth cation independently selected from Na⁺, K⁺, Ca²⁺, and Mg²⁺; provided that first cation, second cation, third cation, and fourth cation are not the same.

In a further aspect, a disclosed modulator composition comprises a first salt having a first cation and a first anion, such that the first cation is selected from Na⁺, K⁺, Ca²⁺, and Mg²⁺; and a first anion is selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₈O₄⁻²), hydrogen adipate C₆H₉O₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof. In a specific aspect, the first anion comprises citrate (C₆H₅O₇⁻³), chloride (Cl⁻), phosphate (PO₄⁻³), carbonate (CO₃⁻²), and combinations thereof. Alternatively, in an aspect, the first anion comprises citrate (C₆H₅O₇⁻³) or the first anion comprises chloride (Cl⁻).

In a further aspect, a disclosed taste modulator composition comprises a first salt having a first cation and a first anion; optionally a second salt having a second cation and a second anion; optionally a third salt having a third cation and a third anion; and optionally a fourth salt having a fourth cation and a fourth anion; the first cation is independently selected from Na⁺, K⁺, Ca²⁺, and Mg²⁺; the second cation is independently selected from Na⁺, K⁺, Ca²⁺, and Mg²⁺; the third cation is independently selected from Na⁺, K⁺, Ca²⁺, and Mg²⁺; and the fourth cation is independently selected from Na⁺, K⁺, Ca²⁺, and Mg²⁺; the first anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₃O₄⁻²), hydrogen adipate C₆H₉O₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻²), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; the second anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₈O₄⁻²), hydrogen adipate C₆H₉O₄⁻¹), lactate (C₃H₅O₃⁻¹), tartrate (C₄H₄O₅²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; the third anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₈O₄⁻²), hydrogen adipate C₆H₉O₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; and the fourth anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₈O₄⁻²), hydrogen adipate C₆H₉O₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof. In some instances, the first cation, the second cation, the third cation, and the fourth cation are not the same. In other instances, some or all of the first cation, the second cation, the third cation, and the fourth cation can be the same, provided that the first anion, the second anion, the third anion, and the fourth anion are not the same.

In a further aspect, a disclosed taste modulator composition comprises a first salt having a first cation and a first anion; a second salt having a second cation and a second anion; optionally a third salt having a third cation and a third anion; and optionally a fourth salt having a fourth cation and a fourth anion; the first cation is independently selected from Na⁺, K⁺, Ca²⁺, and Mg²⁺; the second cation is independently selected from Na⁺, K⁺, Ca²⁺, and Mg²⁺; the third cation is independently selected from Na⁺, K⁺, Ca²⁺, and Mg²⁺; and the fourth cation is independently selected from Na⁺, K⁺, Ca²⁺, and Mg²⁺; the first anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₄⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₈O₄⁻²), hydrogen adipate C₆H₉O₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; the second anion is independently selected from gluconate (C₆H₁₁O₇⁻¹), citrate (CH₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₈O₄⁻²), hydrogen adipate C₆H₉O₄⁻¹), lactate (C₃H₅O₃⁻¹), tartrate (C₄H₄O₅⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; the third anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₆⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₈O₄⁻²), hydrogen adipate C₆H₉O₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; and the fourth anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₈O₄⁻²), hydrogen adipate C₆H₉O₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof. In some instances, the first cation, the second cation, the third cation, and the fourth cation are not the same. In other instances, some or all of the first cation, the second cation, the third cation, and the fourth cation can be the same, provided that the first anion, the second anion, the third anion, and the fourth anion are not the same.

In a further aspect, a disclosed taste modulator composition comprises a first salt having a first cation and a first anion; a second salt having a second cation and a second anion; a third salt having a third cation and a third anion; and optionally a fourth salt having a fourth cation and a fourth anion; the first cation is independently selected from Na⁺, K⁺, Ca²⁺, and Mg²⁺; the second cation is independently selected from Na⁺, K⁺, Ca²⁺, and Mg²⁺; the third cation is independently selected from Na⁺, K⁺, Ca²⁺, and Mg²⁺; and the fourth cation is independently selected from Na⁺, K⁺, Ca²⁺, and Mg²⁺; the first anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₈O₄⁻²), hydrogen adipate C₆H₉O₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; the second anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₈O₄⁻²), hydrogen adipate C₆H₉O₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; the third anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₈O₄⁻²), hydrogen adipate C₆H₉O₄⁻¹), lactate (C₃H₅O₃⁻¹), tartrate (C₄H₄O₅⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; and the fourth anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₈O₄⁻²), hydrogen adipate C₆H₉O₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof. In some instances, the first cation, the second cation, the third cation, and the fourth cation are not the same. In other instances, some or all of the first cation, the second cation, the third cation, and the fourth cation can be the same, provided that the first anion, the second anion, the third anion, and the fourth anion are not the same.

In a further aspect, a disclosed taste modulator composition comprises a first salt having a first cation and a first anion; a second salt having a second cation and a second anion; a third salt having a third cation and a third anion; the first cation is independently selected from Na⁺, K⁺, Ca²⁺, and Mg²⁺; the second cation is independently selected from Na⁺, K⁺, Ca²⁺, and Mg²⁺; the third cation is independently selected from Na⁺, K⁺, Ca²⁺, and Mg²⁺; the first anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₈O₄⁻²), hydrogen adipate C₆H₉O₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; the second anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₆H₆O₄⁻²), hydrogen glutarate (C₅H₇O₄⁻¹), adipate C₆H₃O₄⁻²), hydrogen adipate C₆HO₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; and the third anion is independently selected from gluconate (CH₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₃O₄⁻²), hydrogen adipate C₆HO₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof. In some instances, the first cation, the second cation, the third cation, and the fourth cation are not the same. In other instances, some or all of the first cation, the second cation, the third cation, and the fourth cation can be the same, provided that the first anion, the second anion, the third anion, and the fourth anion are not the same.

In a further aspect, a disclosed taste modulator composition comprises a first salt having a first cation and a first anion; a second salt having a second cation and a second anion; a third salt having a third cation and a third anion; the first cation is K⁺; the second cation is Mg²⁺; the third cation is Ca²⁺; the first anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₆H₀O₄⁻²), hydrogen glutarate (C₅H₇O₄⁻¹), adipate C₆H₃O₄⁻²), hydrogen adipate C₆H₉O₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; the second anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₃O₄⁻²), hydrogen adipate C₆H₉O₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; and the third anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₈O₄⁻²), hydrogen adipate C₆H₉O₄⁻¹), lactate (C₃H₅O₃⁻¹), tartrate (C₄H₄O₅⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof. In some instances, the first cation, the second cation, the third cation, and the fourth cation are not the same. In other instances, some or all of the first cation, the second cation, the third cation, and the fourth cation can be the same, provided that the first anion, the second anion, the third anion, and the fourth anion are not the same.

In a further aspect, a disclosed taste modulator composition comprises a first salt having a first cation and a first anion; a second salt having a second cation and a second anion; a third salt having a third cation and a third anion; the first cation is K⁺; the second cation is Mg²⁺; the third cation is Ca²⁺; the first anion is independently selected from citrate (C₆H₅O₇⁻³) or conjugate acid form thereof, chloride (Cl⁻), sulfate (SO₄⁻²), or bisulfate (HSO₄⁻¹), or combinations thereof; the second anion is independently selected from citrate (C₆H₅O₇⁻³) or conjugate acid form thereof, chloride (Cl⁻), sulfate (SO₄⁻²), or bisulfate (HSO₄⁻¹), or combinations thereof; and the third anion is independently selected from citrate (C₆H₅O₇⁻³) or conjugate acid form thereof, chloride (Cl⁻), sulfate (SO₄⁻²), or bisulfate (HSO₄⁻¹), or combinations thereof. In some instances, the first cation, the second cation, the third cation, and the fourth cation are not the same. In other instances, some or all of the first cation, the second cation, the third cation, and the fourth cation can be the same, provided that the first anion, the second anion, the third anion, and the fourth anion are not the same.

In various aspects, a disclosed taste modulator composition comprises a first taste modulator component comprising a first salt having a first cation selected from Na⁺, K⁺, Ca²⁺, and Mg²⁺ and a first anion; and a second modulator component comprising a second salt having a second cation selected from Na⁺, K⁺, Ca²⁺, and Mg²⁺ and a second anion; such that the first taste modulator component is present at a concentration of from about 0 mM to about 25 mM if the first cation is Na⁺ or K⁺, or at a concentration of from about 0 mM to about 10 mM if the first cation is Ca²⁺ or Mg²⁺; and such that the second taste modulator component is present at a concentration of from about 0 mM to about 25 mM if the second cation is Na⁺ or K⁺, or at a concentration of from about 0 mM to about 10 mM if the second cation is Ca²⁺ or Mg².

In various aspects, a disclosed taste modulator composition comprises a first taste modulator component comprising a first salt having a first cation selected from Na⁺, K⁺, Ca²⁺, and Mg²⁺ and a first anion; and a second modulator component comprising a second salt having a second cation selected from Na⁺, K⁺, Ca²⁺, and Mg²⁺ and a second anion; such that the first taste modulator component is present at a concentration of from about 0 mM to about 25 mM if the first cation is Na⁺ or K⁺, or at a concentration of from about 0 mM to about 5 mM if the first cation is Ca²⁺ or Mg²⁺; and such that the second taste modulator component is present at a concentration of from about 0 mM to about 25 mM if the second cation is Na⁺ or K⁺, or at a concentration of from about 0 mM to about 10 mM if the second cation is Ca²⁺ or Mg²⁺ or K⁺, or at a concentration of from about 0 mM to about 10 mM if the third cation is Ca²⁺ or Mg²⁺.

In various aspects, a disclosed taste modulator composition comprises a first taste modulator component comprising a first salt having a first cation selected from Na⁺, K⁺, Ca²⁺, and Mg²⁺ and a first anion; and a second modulator component comprising a second salt having a second cation selected from Na⁺, K⁺, Ca²⁺, and Mg²⁺ and a second anion; such that the first taste modulator component is present at a concentration of from about 0 mM to about 10 mM if the first cation is Na⁺ or K⁺, or at a concentration of from about 0 mM to about 5 mM if the first cation is Ca²⁺ or Mg²⁺; and such that the second taste modulator component is present at a concentration of from about 0 mM to about 10 mM if the second cation is Na⁺ or K⁺, or at a concentration of from about 0 mM to about 5 mM if the second cation is Ca²⁺ or Mg²⁺.

In various aspects, a disclosed taste modulator composition comprises a first taste modulator component comprising a first salt having a first cation selected from Na⁺, K⁺, Ca²⁺, and Mg²⁺ and a first anion; a second modulator component comprising a second salt having a second cation selected from Na⁺, K⁺, Ca²⁺, and Mg²⁺ and a second anion; such that the first taste modulator component is present at a concentration of from about 0.1 mM to about 25 mM if the first cation is Na⁺ or K⁺, or at a concentration of from about 0.1 mM to about 10 mM if the first cation is Ca²⁺ or Mg²⁺; and such that the second taste modulator component is present at a concentration of from about 0.1 mM to about 25 mM if the second cation is Na⁺ or K⁺, or at a concentration of from about 0.1 mM to about 10 mM if the second cation is Ca²⁺ or Mg²⁺ or Mg²⁺.

In various aspects, a disclosed taste modulator composition comprises a first taste modulator component comprising a first salt having a first cation selected from Na⁺, K⁺, Ca²⁺, and Mg²⁺ and a first anion; a second modulator component comprising a second salt having a second cation selected from Na⁺, K⁺, Ca²⁺, and Mg²⁺ and a second anion; such that the first taste modulator component is present at a concentration of from about 0.1 mM to about 25 mM if the first cation is Na⁺ or K⁺, or at a concentration of from about 0.1 mM to about 5 mM if the first cation is Ca²⁺ or Mg²⁺; and such that the second taste modulator component is present at a concentration of from about 0.1 mM to about 25 mM if the second cation is Na⁺ or K⁺, or at a concentration of from about 0.1 mM to about 10 mM if the second cation is Ca²⁺ or Mg²⁺.

In various aspects, a disclosed taste modulator composition comprises a first taste modulator component comprising a first salt having a first cation selected from Na⁺, K⁺, Ca²⁺, and Mg²⁺ and a first anion; a second modulator component comprising a second salt having a second cation selected from Na⁺, K⁺, Ca²⁺, and Mg²⁺ and a second anion; such that the first taste modulator component is present at a concentration of from about 0.1 mM to about 10 mM if the first cation is Na⁺ or K⁺, or at a concentration of from about 0.1 mM to about 5 mM if the first cation is Ca²⁺ or Mg²⁺; and such that the second taste modulator component is present at a concentration of from about 0.1 mM to about 10 mM if the second cation is Na⁺ or K⁺, or at a concentration of from about 0.1 mM to about 5 mM if the second cation is Ca²⁺ or Mg²⁺.

In various aspects, a disclosed taste modulator composition comprises a first taste modulator component comprising a first salt having a first cation selected from Na⁺, K⁺, Ca²⁺, and Mg²⁺ and a first anion; a second modulator component comprising a second salt having a second cation selected from Na⁺, K⁺, Ca²⁺, and Mg²⁺ and a second anion; such that the first taste modulator component is present at a concentration of from about 1 mM to about 25 mM if the first cation is Na⁺ or K⁺, or at a concentration of from about 1 mM to about 10 mM if the first cation is Ca²⁺ or Mg²⁺; and such that the second taste modulator component is present at a concentration of from about 1 mM to about 25 mM if the second cation is Na⁺ or K⁺, or at a concentration of from about 1 mM to about 10 mM if the second cation is Ca²⁺ or Mg²⁺.

In various aspects, a disclosed taste modulator composition comprises a first taste modulator component comprising a first salt having a first cation selected from Na⁺, K⁺, Ca²⁺, and Mg²⁺ and a first anion; a second modulator component comprising a second salt having a second cation selected from Na⁺, K⁺, Ca²⁺, and Mg²⁺ and a second anion; such that the first taste modulator component is present at a concentration of from about 1 mM to about 25 mM if the first cation is Na⁺ or K⁺, or at a concentration of from about 1 mM to about 5 mM if the first cation is Ca²⁺ or Mg²⁺; and such that the second taste modulator component is present at a concentration of from about 1 mM to about 25 mM if the second cation is Na⁺ or K⁺, or at a concentration of from about 1 mM to about 10 mM if the second cation is Ca²⁺ or Mg²⁺.

In various aspects, a disclosed taste modulator composition comprises a first taste modulator component comprising a first salt having a first cation selected from Na⁺, K⁺, Ca²⁺, and Mg²⁺ and a first anion; a second modulator component comprising a second salt having a second cation selected from Na⁺, K⁺, Ca²⁺, and Mg²⁺ and a second anion; such that the first taste modulator component is present at a concentration of from about 1 mM to about 10 mM if the first cation is Na⁺ or K⁺, or at a concentration of from about 1 mM to about 5 mM if the first cation is Ca²⁺ or Mg²⁺; and such that the second taste modulator component is present at a concentration of from about 1 mM to about 10 mM if the second cation is Na⁺ or K⁺, or at a concentration of from about 1 mM to about 5 mM if the second cation is Ca²⁺ or Mg²⁺.

In various aspects, a disclosed taste modulator composition comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0 mM to about 25 mM if the first cation is Na⁺ or K⁺, or at a concentration of from about 0 mM to about 10 mM if the first cation is Ca²⁺ or Mg²⁺; such that the second taste modulator component is present at a concentration of from about 0 mM to about 25 mM if the second cation is Na⁺ or K⁺, or at a concentration of from about 0 mM to about 10 mM if the second cation is Ca²⁺ or Mg²⁺; and such that the third taste modulator component is present at a concentration of from about 0 mM to about 25 mM if the third cation is Na⁺ or K⁺, or at a concentration of from about 0 mM to about 10 mM if the third cation is Ca²⁺ or Mg²⁺.

In various aspects, a disclosed taste modulator composition comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0 mM to about 25 mM if the first cation is Na⁺ or K⁺, or at a concentration of from about 0 mM to about 5 mM if the first cation is Ca²⁺ or Mg²⁺; such that the second taste modulator component is present at a concentration of from about 0 mM to about 25 mM if the second cation is Na⁺ or K⁺, or at a concentration of from about 0 mM to about 10 mM if the second cation is Ca²⁺ or Mg²⁺; and such that the third taste modulator component is present at a concentration of from about 0 mM to about 25 mM if the third cation is Na⁺ or K⁺, or at a concentration of from about 0 mM to about 10 mM if the third cation is Ca²⁺ or Mg²⁺.

In various aspects, a disclosed taste modulator composition comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0 mM to about 10 mM if the first cation is Na⁺ or K⁺, or at a concentration of from about 0 mM to about 5 mM if the first cation is Ca²⁺ or Mg²⁺; such that the second taste modulator component is present at a concentration of from about 0 mM to about 10 mM if the second cation is Na⁺ or K⁺, or at a concentration of from about 0 mM to about 5 mM if the second cation is Ca²⁺ or Mg²⁺; and such that the third taste modulator component is present at a concentration of from about 0 mM to about 10 mM if the third cation is Na⁺ or K⁺, or at a concentration of from about 0 mM to about 5 mM if the third cation is Ca²⁺ or Mg²⁺.

In various aspects, a disclosed taste modulator composition comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0.1 mM to about 25 mM if the first cation is Na⁺ or K⁺, or at a concentration of from about 0.1 mM to about 10 mM if the first cation is Ca²⁺ or Mg²⁺; such that the second taste modulator component is present at a concentration of from about 0.1 mM to about 25 mM if the second cation is Na⁺ or K⁺, or at a concentration of from about 0.1 mM to about 10 mM if the second cation is Ca²⁺ or Mg²⁺; and such that the third taste modulator component is present at a concentration of from about 0.1 mM to about 25 mM if the third cation is Na⁺ or K⁺, or at a concentration of from about 0.1 mM to about 10 mM if the third cation is Ca²⁺ or Mg²⁺.

In various aspects, a disclosed taste modulator composition comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0.1 mM to about 25 mM if the first cation is Na⁺ or K⁺, or at a concentration of from about 0.1 mM to about 5 mM if the first cation is Ca²⁺ or Mg²⁺; such that the second taste modulator component is present at a concentration of from about 0.1 mM to about 25 mM if the second cation is Na⁺ or K⁺, or at a concentration of from about 0.1 mM to about 10 mM if the second cation is Ca²⁺ or Mg²⁺; and such that the third taste modulator component is present at a concentration of from about 0.1 mM to about 25 mM if the third cation is Na⁺ or K⁺, or at a concentration of from about 0.1 mM to about 10 mM if the third cation is Ca²⁺ or Mg²⁺.

In various aspects, a disclosed taste modulator composition comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0.1 mM to about 10 mM if the first cation is Na⁺ or K⁺, or at a concentration of from about 0.1 mM to about 5 mM if the first cation is Ca²⁺ or Mg²⁺; such that the second taste modulator component is present at a concentration of from about 0.1 mM to about 10 mM if the second cation is Na⁺ or K⁺, or at a concentration of from about 0.1 mM to about 5 mM if the second cation is Ca²⁺ or Mg²⁺; and such that the third taste modulator component is present at a concentration of from about 0.1 mM to about 10 mM if the third cation is Na⁺ or K⁺, or at a concentration of from about 0.1 mM to about 5 mM if the third cation is Ca²⁺ or Mg²⁺.

In various aspects, a disclosed taste modulator composition comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 1 mM to about 25 mM if the first cation is Na⁺ or K⁺, or at a concentration of from about 1 mM to about 10 mM if the first cation is Ca²⁺ or Mg²⁺; such that the second taste modulator component is present at a concentration of from about 1 mM to about 25 mM if the second cation is Na⁺ or K⁺, or at a concentration of from about 1 mM to about 10 mM if the second cation is Ca²⁺ or Mg²⁺; and such that the third taste modulator component is present at a concentration of from about 1 mM to about 25 mM if the third cation is Na⁺ or K⁺, or at a concentration of from about 1 mM to about 10 mM if the third cation is Ca²⁺ or Mg²⁺.

In various aspects, a disclosed taste modulator composition comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 1 mM to about 25 mM if the first cation is Na⁺ or K⁺, or at a concentration of from about 1 mM to about 5 mM if the first cation is Ca²⁺ or Mg²⁺; such that the second taste modulator component is present at a concentration of from about 1 mM to about 25 mM if the second cation is Na⁺ or K⁺, or at a concentration of from about 1 mM to about 10 mM if the second cation is Ca²⁺ or Mg²⁺; and such that the third taste modulator component is present at a concentration of from about 1 mM to about 25 mM if the third cation is Na⁺ or K⁺, or at a concentration of from about 1 mM to about 10 mM if the third cation is Ca²⁺ or Mg²⁺.

In various aspects, a disclosed taste modulator composition comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 1 mM to about 10 mM if the first cation is Na⁺ or K⁺, or at a concentration of from about 1 mM to about 5 mM if the first cation is Ca²⁺ or Mg²⁺; such that the second taste modulator component is present at a concentration of from about 1 mM to about 10 mM if the second cation is Na⁺ or K⁺, or at a concentration of from about 1 mM to about 5 mM if the second cation is Ca²⁺ or Mg²⁺; and such that the third taste modulator component is present at a concentration of from about 1 mM to about 10 mM if the third cation is Na⁺ or K⁺, or at a concentration of from about 1 mM to about 5 mM if the third cation is Ca²⁺ or Mg²⁺.

In various aspects, a disclosed taste modulator composition comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0 mM to about 25 mM; and such that each of the second taste modulator component and the third modulator component are independently present at a concentration of from about 0 mM to about 10 mM.

In various aspects, a disclosed taste modulator composition comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0 mM to about 25 mM; and such that each of the second taste modulator component and the third modulator component are independently present at a concentration of from about 0 mM to about 5 mM.

In various aspects, a disclosed taste modulator composition comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0 mM to about 10 mM; and such that each of the second taste modulator component and the third modulator component are independently present at a concentration of from about 0 mM to about 5 mM.

In various aspects, a disclosed taste modulator composition comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0.1 mM to about 25 mM; and such that each of the second taste modulator component and the third modulator component are independently present at a concentration of from about 0.1 mM to about 10 mM.

In various aspects, a disclosed taste modulator composition comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0.1 mM to about 25 mM; and such that each of the second taste modulator component and the third modulator component are independently present at a concentration of from about 0.1 mM to about 5 mM.

In various aspects, a disclosed taste modulator composition comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0.1 mM to about 10 mM; and such that each of the second taste modulator component and the third modulator component are independently present at a concentration of from about 0.1 mM to about 5 mM.

In various aspects, a disclosed taste modulator composition comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 1 mM to about 25 mM; and such that each of the second taste modulator component and the third modulator component are independently present at a concentration of from about 1 mM to about 10 mM.

In various aspects, a disclosed taste modulator composition comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 1 mM to about 25 mM; and such that each of the second taste modulator component and the third modulator component are independently present at a concentration of from about 1 mM to about 5 mM.

In various aspects, a disclosed taste modulator composition comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 1 mM to about 10 mM; and such that each of the second taste modulator component and the third modulator component are independently present at a concentration of from about 1 mM to about 5 mM.

In various aspects, a disclosed taste modulator composition comprises a first taste modulator component comprising a first salt having a first cation selected from Na⁺, K⁺, Ca²⁺, and Mg²⁺ and a first anion selected from citrate (C₆H₅O₇⁻³), chloride (Cl⁻), phosphate (PO₄⁻³), carbonate (CO₃⁻²), sulfate (SO₄⁻²), and combinations thereof; a second modulator component comprising a second salt having a second cation selected from Na⁺, K⁺, Ca²⁺, and Mg²⁺ and a second anion selected from citrate (C₆H₅O₇⁻³), chloride (Cl⁻), phosphate (PO₄⁻³), carbonate (CO₃⁻²), sulfate (SO₄⁻²), and combinations thereof; and a third modulator component comprising a third salt having a third cation selected from Na⁺, K⁺, Ca²⁺, and Mg²⁺ and a third anion selected from citrate (C₆H₅O₇⁻³), chloride (Cl⁻), phosphate (PO₄⁻³), carbonate (CO₃⁻²), sulfate (SO₄⁻²), and combinations thereof; such that the first taste modulator component is present at a concentration of from about 0 mM to about 10 mM; and such that each of the second taste modulator component and the third modulator component are independently present at a concentration of from about 0 mM to about 5 mM.

In various aspects, a disclosed taste modulator composition comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0.1 mM to about 10 mM; and such that each of the second taste modulator component and the third modulator component are independently present at a concentration of from about 0.1 mM to about 5 mM.

In various aspects, a disclosed taste modulator composition comprises a first taste modulator component comprising a first salt having a first cation selected from Na⁺, K⁺, Ca²⁺, and Mg²⁺ and a first anion selected from citrate (C₆H₅O₇⁻³), chloride (Cl⁻), phosphate (PO₄⁻³), carbonate (CO₃⁻²), sulfate (SO₄⁻²), and combinations thereof; a second modulator component comprising a second salt having a second cation selected from Na⁺, K⁺, Ca²⁺, and Mg²⁺ and a second anion selected from citrate (C₆H₅O₇⁻³), chloride (Cl⁻), phosphate (PO₄⁻³), carbonate (CO₃⁻²), sulfate (SO₄⁻²), and combinations thereof; and a third modulator component comprising a third salt having a third cation selected from Na⁺, K⁺, Ca²⁺, and Mg²⁺ and a third anion selected from citrate (C₆H₅O₇⁻³), chloride (Cl⁻), phosphate (PO₄⁻³), carbonate (CO₃⁻²), sulfate (SO₄⁻²), and combinations thereof; such that the first taste modulator component is present at a concentration of from about 0.1 mM to about 10 mM; and such that each of the second taste modulator component and the third modulator component are independently present at a concentration of from about 0.1 mM to about 5 mM.

In various aspects, a disclosed taste modulator composition comprises a first taste modulator component comprising a first salt having a first cation comprising K⁺; a second modulator component comprising a second salt having a second cation comprising Mg²⁺; and a third modulator component comprising a third salt having a third cation comprising Ca²⁺; such that the first taste modulator component is present at a concentration of from about 0 mM to about 10 mM; and such that each of the second taste modulator component and the third modulator component are independently present at a concentration of from about 0 mM to about 5 mM.

In various aspects, a disclosed taste modulator composition comprises a first taste modulator component comprising a first salt having a first cation comprising K⁺ and a first anion selected from citrate (C₆H₅O₇⁻³), chloride (Cl⁻), phosphate (PO₄⁻³), carbonate (CO₃⁻²), sulfate (SO₄⁻²), and combinations thereof; a second modulator component comprising a second salt having a second cation comprising Mg²⁺ and a second anion selected from citrate (C₆H₅O₇⁻³), chloride (Cl⁻), phosphate (PO₄⁻³), carbonate (CO₃⁻²), sulfate (SO₄⁻²), and combinations thereof; and a third modulator component comprising a third salt having a third cation comprising Ca²⁺ and a third anion selected from citrate (CH₅O₇⁻³), chloride (Cl⁻), phosphate (PO₄⁻³), carbonate (CO₃⁻²), sulfate (SO₄⁻²), and combinations thereof; such that the first taste modulator component is present at a concentration of from about 0 mM to about 10 mM; and such that each of the second taste modulator component and the third modulator component are independently present at a concentration of from about 0 mM to about 5 mM.

In various aspects, a disclosed taste modulator composition comprises a first taste modulator component comprising a first salt having a first cation comprising K⁺; a second modulator component comprising a second salt having a second cation comprising Mg²⁺; and a third modulator component comprising a third salt having a third cation comprising Ca²⁺; such that the first taste modulator component is present at a concentration of from about 0.1 mM to about 10 mM; and such that each of the second taste modulator component and the third modulator component are independently present at a concentration of from about 0.1 mM to about 5 mM.

In various aspects, a disclosed taste modulator composition comprises a first taste modulator component comprising a first salt having a first cation comprising K⁺ and a first anion selected from citrate (C₆H₅O₇⁻³), chloride (Cl⁻), phosphate (PO₄⁻³), carbonate (CO₃⁻²), sulfate (SO₄⁻²), and combinations thereof; a second modulator component comprising a second salt having a second cation comprising Mg²⁺ and a second anion selected from citrate (C₆H₅O₇⁻³), chloride (Cl⁻), phosphate (PO₄⁻³), carbonate (CO₃⁻²), sulfate (SO₄⁻²), and combinations thereof; and a third modulator component comprising a third salt having a third cation comprising Ca²⁺ and a third anion selected from citrate (C₆H₅O₇⁻³), chloride (Cl⁻), phosphate (PO₄⁻³), carbonate (CO₃⁻²), sulfate (SO₄⁻²), and combinations thereof; such that the first taste modulator component is present at a concentration of from about 0.1 mM to about 10 mM; and such that each of the second taste modulator component and the third modulator component are independently present at a concentration of from about 0.1 mM to about 5 mM.

In various aspects, the total concentration of the first modulator component, the second modulator component, the third modulator component, and the fourth modulator component taken together can be from about 0.1 mM to about 30 mM. For example, each of the first modulator component, the second modulator component, the third modulator component, and the fourth modulator component are independently present a concentration of from about 0 mM to about 10 mM, provided that the sum of concentrations of the first modulator component, the second modulator component, the third modulator component, and the fourth modulator component is less than about 30 mM. In a further aspect, the total concentration of the first modulator component, the second modulator component, the third modulator component, and the fourth modulator component taken together can be from about 0.1 mM to about 30 mM, about 0.2 mM to about 30 mM, about 0.3 mM to about 30 mM, about 0.4 mM to about 30 mM, about 0.5 mM to about 30 mM, about 0.6 mM to about 30 mM, about 0.7 mM to about 30 mM, about 0.8 mM to about 30 mM, about 0.9 mM to about 30 mM, about 1.0 mM to about 30 mM, about 0.1 mM to about 25 mM, about 0.2 mM to about 25 mM, about 0.3 mM to about 25 mM, about 0.4 mM to about 25 mM, about 0.5 mM to about 25 mM, about 0.6 mM to about 25 mM, about 0.7 mM to about 25 mM, about 0.8 mM to about 25 mM, about 0.9 mM to about 25 mM, about 1.0 mM to about 25 mM, about 0.1 mM to about 20 mM, about 0.2 mM to about 20 mM, about 0.3 mM to about 20 mM, about 0.4 mM to about 20 mM, about 0.5 mM to about 20 mM, about 0.6 mM to about 20 mM, about 0.7 mM to about 20 mM, about 0.8 mM to about 20 mM, about 0.9 mM to about 20 mM, about 1.0 mM to about 20 mM, about 0.1 mM to about 15 mM, about 0.2 mM to about 15 mM, about 0.3 mM to about 15 mM, about 0.4 mM to about 15 mM, about 0.5 mM to about 15 mM, about 0.6 mM to about 15 mM, about 0.7 mM to about 15 mM, about 0.8 mM to about 15 mM, about 0.9 mM to about 15 mM, about 1.0 mM to about 15 mM, about 0.1 mM to about 10 mM, about 0.2 mM to about 10 mM, about 0.3 mM to about 10 mM, about 0.4 mM to about 10 mM, about 0.5 mM to about 10 mM, about 0.6 mM to about 10 mM, about 0.7 mM to about 10 mM, about 0.8 mM to about 10 mM, about 0.9 mM to about 10 mM, about 1.0 mM to about 10 mM; or a concentration or set of concentrations within the foregoing concentration ranges; or a sub-range of any of the foregoing concentration ranges.

In a further aspect, the concentration of the first modulator component can be from about 0.1 mM to about 30 mM, about 0.2 mM to about 30 mM, about 0.3 mM to about 30 mM, about 0.4 mM to about 30 mM, about 0.5 mM to about 30 mM, about 0.6 mM to about 30 mM, about 0.7 mM to about 30 mM, about 0.8 mM to about 30 mM, about 0.9 mM to about 30 mM, about 1.0 mM to about 30 mM, about 0.1 mM to about 25 mM, about 0.2 mM to about 25 mM, about 0.3 mM to about 25 mM, about 0.4 mM to about 25 mM, about 0.5 mM to about 25 mM, about 0.6 mM to about 25 mM, about 0.7 mM to about 25 mM, about 0.8 mM to about 25 mM, about 0.9 mM to about 25 mM, about 1.0 mM to about 25 mM, about 0.1 mM to about 20 mM, about 0.2 mM to about 20 mM, about 0.3 mM to about 20 mM, about 0.4 mM to about 20 mM, about 0.5 mM to about 20 mM, about 0.6 mM to about 20 mM, about 0.7 mM to about 20 mM, about 0.8 mM to about 20 mM, about 0.9 mM to about 20 mM, about 1.0 mM to about 20 mM, about 0.1 mM to about 15 mM, about 0.2 mM to about 15 mM, about 0.3 mM to about 15 mM, about 0.4 mM to about 15 mM, about 0.5 mM to about 15 mM, about 0.6 mM to about 15 mM, about 0.7 mM to about 15 mM, about 0.8 mM to about 15 mM, about 0.9 mM to about 15 mM, about 1.0 mM to about 15 mM, about 0.1 mM to about 10 mM, about 0.2 mM to about 10 mM, about 0.3 mM to about 10 mM, about 0.4 mM to about 10 mM, about 0.5 mM to about 10 mM, about 0.6 mM to about 10 mM, about 0.7 mM to about 10 mM, about 0.8 mM to about 10 mM, about 0.9 mM to about 10 mM, about 1.0 mM to about 10 mM, about 0.1 mM to about 9 mM, about 0.2 mM to about 9 mM, about 0.3 mM to about 9 mM, about 0.4 mM to about 9 mM, about 0.5 mM to about 9 mM, about 0.6 mM to about 9 mM, about 0.7 mM to about 9 mM, about 0.8 mM to about 9 mM, about 0.9 mM to about 9 mM, about 1.0 mM to about 9 mM, about 0.1 mM to about 8 mM, about 0.2 mM to about 8 mM, about 0.3 mM to about 8 mM, about 0.4 mM to about 8 mM, about 0.5 mM to about 8 mM, about 0.6 mM to about 8 mM, about 0.7 mM to about 8 mM, about 0.8 mM to about 8 mM, about 0.9 mM to about 8 mM, about 1.0 mM to about 8 mM, about 0.1 mM to about 7 mM, about 0.2 mM to about 7 mM, about 0.3 mM to about 7 mM, about 0.4 mM to about 7 mM, about 0.5 mM to about 7 mM, about 0.6 mM to about 7 mM, about 0.7 mM to about 7 mM, about 0.8 mM to about 7 mM, about 0.9 mM to about 7 mM, about 1.0 mM to about 7 mM, about 0.1 mM to about 6 mM, about 0.2 mM to about 6 mM, about 0.3 mM to about 6 mM, about 0.4 mM to about 6 mM, about 0.5 mM to about 6 mM, about 0.6 mM to about 6 mM, about 0.7 mM to about 6 mM, about 0.8 mM to about 6 mM, about 0.9 mM to about 6 mM, about 1.0 mM to about 6 mM, about 0.1 mM to about 5 mM, about 0.2 mM to about 5 mM, about 0.3 mM to about 5 mM, about 0.4 mM to about 5 mM, about 0.5 mM to about 5 mM, about 0.6 mM to about 5 mM, about 0.7 mM to about 5 mM, about 0.8 mM to about 5 mM, about 0.9 mM to about 5 mM, about 1.0 mM to about 5 mM, about 0.1 mM to about 4 mM, about 0.2 mM to about 4 mM, about 0.3 mM to about 4 mM, about 0.4 mM to about 4 mM, about 0.5 mM to about 4 mM, about 0.6 mM to about 4 mM, about 0.7 mM to about 4 mM, about 0.8 mM to about 4 mM, about 0.9 mM to about 4 mM, about 1.0 mM to about 4 mM, about 0.1 mM to about 3 mM, about 0.2 mM to about 3 mM, about 0.3 mM to about 3 mM, about 0.4 mM to about 3 mM, about 0.5 mM to about 3 mM, about 0.6 mM to about 3 mM, about 0.7 mM to about 3 mM, about 0.8 mM to about 3 mM, about 0.9 mM to about 3 mM, about 1.0 mM to about 3 mM; or a concentration or set of concentrations within the foregoing concentration ranges; or a sub-range of any of the foregoing concentration ranges.

In a further aspect, the concentration of the second modulator component can be from about 0.1 mM to about 30 mM, about 0.2 mM to about 30 mM, about 0.3 mM to about 30 mM, about 0.4 mM to about 30 mM, about 0.5 mM to about 30 mM, about 0.6 mM to about 30 mM, about 0.7 mM to about 30 mM, about 0.8 mM to about 30 mM, about 0.9 mM to about 30 mM, about 1.0 mM to about 30 mM, about 0.1 mM to about 25 mM, about 0.2 mM to about 25 mM, about 0.3 mM to about 25 mM, about 0.4 mM to about 25 mM, about 0.5 mM to about 25 mM, about 0.6 mM to about 25 mM, about 0.7 mM to about 25 mM, about 0.8 mM to about 25 mM, about 0.9 mM to about 25 mM, about 1.0 mM to about 25 mM, about 0.1 mM to about 20 mM, about 0.2 mM to about 20 mM, about 0.3 mM to about 20 mM, about 0.4 mM to about 20 mM, about 0.5 mM to about 20 mM, about 0.6 mM to about 20 mM, about 0.7 mM to about 20 mM, about 0.8 mM to about 20 mM, about 0.9 mM to about 20 mM, about 1.0 mM to about 20 mM, about 0.1 mM to about 15 mM, about 0.2 mM to about 15 mM, about 0.3 mM to about 15 mM, about 0.4 mM to about 15 mM, about 0.5 mM to about 15 mM, about 0.6 mM to about 15 mM, about 0.7 mM to about 15 mM, about 0.8 mM to about 15 mM, about 0.9 mM to about 15 mM, about 1.0 mM to about 15 mM, about 0.1 mM to about 10 mM, about 0.2 mM to about 10 mM, about 0.3 mM to about 10 mM, about 0.4 mM to about 10 mM, about 0.5 mM to about 10 mM, about 0.6 mM to about 10 mM, about 0.7 mM to about 10 mM, about 0.8 mM to about 10 mM, about 0.9 mM to about 10 mM, about 1.0 mM to about 10 mM, about 0.1 mM to about 9 mM, about 0.2 mM to about 9 mM, about 0.3 mM to about 9 mM, about 0.4 mM to about 9 mM, about 0.5 mM to about 9 mM, about 0.6 mM to about 9 mM, about 0.7 mM to about 9 mM, about 0.8 mM to about 9 mM, about 0.9 mM to about 9 mM, about 1.0 mM to about 9 mM, about 0.1 mM to about 8 mM, about 0.2 mM to about 8 mM, about 0.3 mM to about 8 mM, about 0.4 mM to about 8 mM, about 0.5 mM to about 8 mM, about 0.6 mM to about 8 mM, about 0.7 mM to about 8 mM, about 0.8 mM to about 8 mM, about 0.9 mM to about 8 mM, about 1.0 mM to about 8 mM, about 0.1 mM to about 7 mM, about 0.2 mM to about 7 mM, about 0.3 mM to about 7 mM, about 0.4 mM to about 7 mM, about 0.5 mM to about 7 mM, about 0.6 mM to about 7 mM, about 0.7 mM to about 7 mM, about 0.8 mM to about 7 mM, about 0.9 mM to about 7 mM, about 1.0 mM to about 7 mM, about 0.1 mM to about 6 mM, about 0.2 mM to about 6 mM, about 0.3 mM to about 6 mM, about 0.4 mM to about 6 mM, about 0.5 mM to about 6 mM, about 0.6 mM to about 6 mM, about 0.7 mM to about 6 mM, about 0.8 mM to about 6 mM, about 0.9 mM to about 6 mM, about 1.0 mM to about 6 mM, about 0.1 mM to about 5 mM, about 0.2 mM to about 5 mM, about 0.3 mM to about 5 mM, about 0.4 mM to about 5 mM, about 0.5 mM to about 5 mM, about 0.6 mM to about 5 mM, about 0.7 mM to about 5 mM, about 0.8 mM to about 5 mM, about 0.9 mM to about 5 mM, about 1.0 mM to about 5 mM, about 0.1 mM to about 4 mM, about 0.2 mM to about 4 mM, about 0.3 mM to about 4 mM, about 0.4 mM to about 4 mM, about 0.5 mM to about 4 mM, about 0.6 mM to about 4 mM, about 0.7 mM to about 4 mM, about 0.8 mM to about 4 mM, about 0.9 mM to about 4 mM, about 1.0 mM to about 4 mM, about 0.1 mM to about 3 mM, about 0.2 mM to about 3 mM, about 0.3 mM to about 3 mM, about 0.4 mM to about 3 mM, about 0.5 mM to about 3 mM, about 0.6 mM to about 3 mM, about 0.7 mM to about 3 mM, about 0.8 mM to about 3 mM, about 0.9 mM to about 3 mM, about 1.0 mM to about 3 mM; or a concentration or set of concentrations within the foregoing concentration ranges; or a sub-range of any of the foregoing concentration ranges.

In a further aspect, the concentration of the third modulator component can be from about 0.1 mM to about 30 mM, about 0.2 mM to about 30 mM, about 0.3 mM to about 30 mM, about 0.4 mM to about 30 mM, about 0.5 mM to about 30 mM, about 0.6 mM to about 30 mM, about 0.7 mM to about 30 mM, about 0.8 mM to about 30 mM, about 0.9 mM to about 30 mM, about 1.0 mM to about 30 mM, about 0.1 mM to about 25 mM, about 0.2 mM to about 25 mM, about 0.3 mM to about 25 mM, about 0.4 mM to about 25 mM, about 0.5 mM to about 25 mM, about 0.6 mM to about 25 mM, about 0.7 mM to about 25 mM, about 0.8 mM to about 25 mM, about 0.9 mM to about 25 mM, about 1.0 mM to about 25 mM, about 0.1 mM to about 20 mM, about 0.2 mM to about 20 mM, about 0.3 mM to about 20 mM, about 0.4 mM to about 20 mM, about 0.5 mM to about 20 mM, about 0.6 mM to about 20 mM, about 0.7 mM to about 20 mM, about 0.8 mM to about 20 mM, about 0.9 mM to about 20 mM, about 1.0 mM to about 20 mM, about 0.1 mM to about 15 mM, about 0.2 mM to about 15 mM, about 0.3 mM to about 15 mM, about 0.4 mM to about 15 mM, about 0.5 mM to about 15 mM, about 0.6 mM to about 15 mM, about 0.7 mM to about 15 mM, about 0.8 mM to about 15 mM, about 0.9 mM to about 15 mM, about 1.0 mM to about 15 mM, about 0.1 mM to about 10 mM, about 0.2 mM to about 10 mM, about 0.3 mM to about 10 mM, about 0.4 mM to about 10 mM, about 0.5 mM to about 10 mM, about 0.6 mM to about 10 mM, about 0.7 mM to about 10 mM, about 0.8 mM to about 10 mM, about 0.9 mM to about 10 mM, about 1.0 mM to about 10 mM, about 0.1 mM to about 9 mM, about 0.2 mM to about 9 mM, about 0.3 mM to about 9 mM, about 0.4 mM to about 9 mM, about 0.5 mM to about 9 mM, about 0.6 mM to about 9 mM, about 0.7 mM to about 9 mM, about 0.8 mM to about 9 mM, about 0.9 mM to about 9 mM, about 1.0 mM to about 9 mM, about 0.1 mM to about 8 mM, about 0.2 mM to about 8 mM, about 0.3 mM to about 8 mM, about 0.4 mM to about 8 mM, about 0.5 mM to about 8 mM, about 0.6 mM to about 8 mM, about 0.7 mM to about 8 mM, about 0.8 mM to about 8 mM, about 0.9 mM to about 8 mM, about 1.0 mM to about 8 mM, about 0.1 mM to about 7 mM, about 0.2 mM to about 7 mM, about 0.3 mM to about 7 mM, about 0.4 mM to about 7 mM, about 0.5 mM to about 7 mM, about 0.6 mM to about 7 mM, about 0.7 mM to about 7 mM, about 0.8 mM to about 7 mM, about 0.9 mM to about 7 mM, about 1.0 mM to about 7 mM, about 0.1 mM to about 6 mM, about 0.2 mM to about 6 mM, about 0.3 mM to about 6 mM, about 0.4 mM to about 6 mM, about 0.5 mM to about 6 mM, about 0.6 mM to about 6 mM, about 0.7 mM to about 6 mM, about 0.8 mM to about 6 mM, about 0.9 mM to about 6 mM, about 1.0 mM to about 6 mM, about 0.1 mM to about 5 mM, about 0.2 mM to about 5 mM, about 0.3 mM to about 5 mM, about 0.4 mM to about 5 mM, about 0.5 mM to about 5 mM, about 0.6 mM to about 5 mM, about 0.7 mM to about 5 mM, about 0.8 mM to about 5 mM, about 0.9 mM to about 5 mM, about 1.0 mM to about 5 mM, about 0.1 mM to about 4 mM, about 0.2 mM to about 4 mM, about 0.3 mM to about 4 mM, about 0.4 mM to about 4 mM, about 0.5 mM to about 4 mM, about 0.6 mM to about 4 mM, about 0.7 mM to about 4 mM, about 0.8 mM to about 4 mM, about 0.9 mM to about 4 mM, about 1.0 mM to about 4 mM, about 0.1 mM to about 3 mM, about 0.2 mM to about 3 mM, about 0.3 mM to about 3 mM, about 0.4 mM to about 3 mM, about 0.5 mM to about 3 mM, about 0.6 mM to about 3 mM, about 0.7 mM to about 3 mM, about 0.8 mM to about 3 mM, about 0.9 mM to about 3 mM, about 1.0 mM to about 3 mM; or a concentration or set of concentrations within the foregoing concentration ranges; or a sub-range of any of the foregoing concentration ranges.

The disclosed taste modulator compositions can be used at a suitable pH, e.g. a pH of from about pH 2 to about pH 9. In some instances, it may be desirable, e.g., to optimize a Taste Quality Metric such as sweetness linger, and/or body/mouthfeel, to use a lower pH, such as a pH of from about pH 2 to about pH 5, about pH 2 to about pH 4.5, about pH 2.0 to about pH 4.0, about pH 2.0 to about pH 3.9, about pH 2.0 to about pH 3.8, about pH 2.0 to about pH 3.7, about pH 2.0 to about pH 3.6, about pH 2.0 to about pH 3.5, about pH 2.0 to about pH 3.4, about pH 2.0 to about pH 3.3, about pH 2.0 to about pH 3.2, about pH 2.0 to about pH 3.1, about pH 2.0 to about pH 3.0, about pH 2.1 to about pH 4.0, about pH 2.1 to about pH 3.9, about pH 2.1 to about pH 3.8, about pH 2.1 to about pH 3.7, about pH 2.1 to about pH 3.6, about pH 2.1 to about pH 3.5, about pH 2.1 to about pH 3.4, about pH 2.1 to about pH 3.3, about pH 2.1 to about pH 3.2, about pH 2.1 to about pH 3.1, about pH 2.1 to about pH 3.0, about pH 2.2 to about pH 4.0, about pH 2.2 to about pH 3.9, about pH 2.2 to about pH 3.8, about pH 2.2 to about pH 3.7, about pH 2.2 to about pH 3.6, about pH 2.2 to about pH 3.5, about pH 2.2 to about pH 3.4, about pH 2.2 to about pH 3.3, about pH 2.2 to about pH 3.2, about pH 2.2 to about pH 3.1, about pH 2.2 to about pH 3.0, about pH 2.3 to about pH 4.0, about pH 2.3 to about pH 3.9, about pH 2.3 to about pH 3.8, about pH 2.3 to about pH 3.7, about pH 2.3 to about pH 3.6, about pH 2.3 to about pH 3.5, about pH 2.3 to about pH 3.4, about pH 2.3 to about pH 3.3, about pH 2.3 to about pH 3.2, about pH 2.3 to about pH 3.1, about pH 2.3 to about pH 3.0, about pH 2.4 to about pH 4.0, about pH 2.4 to about pH 3.9, about pH 2.4 to about pH 3.8, about pH 2.4 to about pH 3.7, about pH 2.4 to about pH 3.6, about pH 2.4 to about pH 3.5, about pH 2.4 to about pH 3.4, about pH 2.4 to about pH 3.3, about pH 2.4 to about pH 3.2, about pH 2.4 to about pH 3.1, about pH 2.4 to about pH 3.0, about pH 2.5 to about pH 4.0, about pH 2.5 to about pH 3.9, about pH 2.5 to about pH 3.8, about pH 2.5 to about pH 3.7, about pH 2.5 to about pH 3.6, about pH 2.5 to about pH 3.5, about pH 2.5 to about pH 3.4, about pH 2.5 to about pH 3.3, about pH 2.5 to about pH 3.2, about pH 2.5 to about pH 3.1, about pH 2.5 to about pH 3.0; or any pH value or sub-range within the foregoing ranges.

### Calcium Sensing Receptor (CaSR)

It has been reported that CaSR can be activated by both Ca²⁺ salts and Mg²⁺ salts as well as a large number of other agonists (e.g., see Spurney, R. F., et al. Kidney Int. 1999 May; 55(5):1750-8; and Breitwieser, G. E., et al. Cell Calcium. 2004 March; 35(3):209-16). The CaSR belongs to class C of the seven-transmembrane receptors (G protein-coupled receptor; GPCR). The cloning of the gene for the calcium receptor was reported in 1993 (Nature, 1993 Dec. 9; 366(6455):575-80). The calcium receptor is known to cause various cellular responses through elevation of the intracellular calcium levels, etc., when activated with calcium, etc. The sequence of the human calcium receptor gene is registered with GenBank (Accession No. NM_000388), and is well conserved among many animal species. The “calcium receptor activity” is when binding of a substrate to the calcium receptor activates the guanine nucleotide binding protein and, as a result, transmits one or more signals. Without wishing to be bound by a particular theory, it is possible that the disclosed taste modulator compositions act in part via activation of the calcium sensing receptor (CaSR).

Further, without wishing to be bound by a particular theory, it is possible that the disclosed taste modulator compositions, acting on the CaSR, may be involved in kokumi taste. In a 2012 paper (Maruyama et al., PLoS ONE, 2012, 7(4): e34489) the activity of the CaSR in taste bud cells was found to be associated with a taste which they refer to as “kokumi taste”. In the field of food chemistry and biochemistry, substances having specific tastes have been used for many years. In particular, substances having the five basic tastes, namely, sweet, salty, sour, bitter, and umami (a delicious taste) have been widely used as seasonings. Substances which enhance these basic tastes have also been widely used. One taste that does not fall within these five basic tastes is “kokumi”. Kokumi means a taste that is not one of the five basic tastes. Kokumi is a taste that not only enhances the five basic tastes but also enhances the marginal tastes of the basic tastes, such as thickness, growth (mouthfulness), continuity, and harmony. Several methods for imparting kokumi have been reported so far. Substances that have been reported to impart kokumi include glutathione (e.g., Japanese Patent No. 1464928), heated products of gelatin and tropomyosin (e.g., Japanese Patent Laid-open Publication (KOKAI) No. 10-276709), sulfone group-containing compounds (e.g., Japanese Patent Laid-open Publication (KOKAI) No. 8-289760), a peptide containing the Asn-His sequence (e.g., WO2004/096836), and so forth.

Accordingly, without wishing to be bound by a particular theory, improvement in the Taste Quality metric by the disclosed taste modulator compositions herein is via kokumi taste mediated at least in part via the CaSR.

In various aspects, a suitable additional CaSR modulator, e.g., a CaSR agonist, positive allosteric modulator, or combinations thereof, can be used in the various disclosed compositions.

For example, a suitable additional CaSR modulator can be used to replace one or more of the taste modulator component, e.g., a first salt, a second salt, and the like. Alternatively, a suitable additional CaSR modulator can be an optional additional component in a disclosed taste modulator composition and by addition thereof decrease the amount of the taste modulator component, e.g., a first salt, a second salt, and the like, used compared to a composition that does not comprise a suitable additional CaSR modulator. Although it is understood that a taste modulator component, taste modulator component, e.g., a first salt, a second salt, and the like, may act at or bind to the CaSR, in the present context, “the suitable additional CaSR modulator” is understood to be a salt, compound, or molecular entity other than a first salt, second salt, third salt, or fourth salt as described herein above. That is, a suitable additional CaSR modulator can be any known CaSR agonist, CaSR positive allosteric modulator, or combination thereof—as known to the skilled artisan—that is a disclosed first salt, second salt, third salt, or fourth salt.

Exemplary, but non-limiting suitable additional CaSR modulators include creatine, spermine, spermidine, putrescine, L-glutathione, neomycin, poly-L-arginine, cinacalcet, calindol, an aluminum salt, an iron salt, a gadolinium salt, a zinc salt, a strontium salt, or combinations thereof. Further exemplary, but non-limiting suitable CaSR modulator includes a peptide that is acts at the CaSR such as a di-, tri-, and tetra-peptide such as Glu-Asp, Glu-Glu, Asp-Glu, Thr-Glu, Asp-Glu-Ser, Glu-Gly-Ser, Asp-Asp-Asp-Asp, and others as known to the skilled artisan. The peptide can be a suitable di-peptide up to an octapeptide, such as Lys-Gly-Asp-Glu-Glu-Ser-Leu-Ala. In some aspects, the peptide can be γ-glutamyl peptide that is a di-peptide to an octapeptide, i.e., a peptide of 2-8 amino acids having a γ-glutamyl residue, including, but not limited to γ-Glu-Ala, γ-Glu-Val, γ-Glu-Cys, γ-Glu-Abu-Gly (Abu=alphabutyric acid), γ-Glu-Val-Gly, γ-Glu-Ser, γ-Glu-Thr, γ-Glu-Glu, and others.

Further exemplary, but non-limiting suitable additional CaSR modulator includes a polyamine such as an organic compound having two or more primary amino groups, e.g., putrescine, cadaverine, spermidine, spermine, or combinations thereof. The polyamine can be a branched or cyclic polyamine. In some instances, it may be preferable to use a cyclic polyamine.

Further exemplary, but non-limiting suitable additional CaSR modulator includes an aminoglycoside antibiotics such as amikacin, gentamicin, kanamycin, neomycin, netilmicin, paromomycin, streptomycin, tobramycin, apramycin, or combinations thereof.

Further exemplary, but non-limiting suitable additional CaSR modulator includes an allosterically active L-amino acid such as an aromatic or small aliphatic L-amino acid, such as Phe, Tyr, Trp, Gly, Ala, Val, Leu, lie, or combinations thereof.

The present disclosure further provides methods for optimizing and modifying the disclosed taste modulator compositions by assessing the activity of same to modulate the activity and/or expression of a CaSR. For example, and not by way of limitation, the disclosed taste modulator compositions can act, in part, as agonists of a calcium-sensing receptor. The presently disclosed subject matter provides in silico and in vitro methods for identifying compounds that modulate the activity and/or expression of a CaSR, disclosed above.

In certain aspects, the method can be an in vitro method for assessing the effect of a disclosed taste modulator composition in modulating the activity and/or expression of a CaSR.

The CaSR for use in the presently disclosed methods can include isolated or recombinant calcium-sensing receptors or cells expressing a calcium-sensing receptor, e.g., a CaSR having an amino acid sequence corresponding to a described CaSR, e.g., GenBank Accession No. NM_000388. In certain aspects, the calcium-sensing receptor for use in the disclosed methods can have the amino acid sequence corresponding to a described CaSR, e.g., GenBank Accession No. NM_000388, or a fragment or variant thereof. In certain aspects, the calcium-sensing receptor for use in the disclosed method can have at least 85%, at least 90%, at least 91%, at least 92%, at least 93%, at least 94%, at least 95%, at least 96%, at least 97%, at least 98% or at least 99% identity to the amino acid sequence corresponding to a described CaSR, e.g., GenBank Accession No. NM_000388, or a fragment or variant thereof. In some aspects, the calcium-sensing receptor for use in the disclosed method can have the nucleotide sequence corresponding to a described CaSR, e.g., GenBank Accession No. NM_000388, or a fragment or variant thereof. In certain aspects, the calcium-sensing receptor for use in the presently disclosed subject matter can include a receptor comprising a nucleotide sequence having at least 85%, at least 90%, at least 91%, at least 92%, at least 93%, at least 94%, at least 95%, at least 96%, at least 97%, at least 98% or at least 99% identity to a nucleic acid sequence corresponding to a described CaSR, e.g., GenBank Accession No. NM_000388, or a fragment or variant thereof.

In certain aspects, the method for assessing the effect of a disclosed taste modulator composition that modulates the activity and/or expression of a calcium-sensing receptor comprises measuring the biological activity of a calcium-sensing receptor in the absence and/or presence of a taste modulator composition. In certain aspects, the method can include measuring the biological activity of a calcium-sensing receptor in the presence of varying concentrations of a disclosed taste modulator composition. The method can further include identifying the taste modulator compositions that result in a modulation of the activity and/or expression of the calcium-sensing receptor compared to the activity and/or expression of the calcium-sensing receptor in the absence of the taste modulator composition.

In certain aspects, a disclosed taste modulator composition can increase the biological activity of a calcium-sensing receptor by at least about 5%, 10%, 15%, 20%, 25%, 30%, 35%, 40%, 45%, 50%, 55%, 60%, 65%, 70%, 75%, 80%, 85%, 90%, 95%, 100%, or more, compared to the biological activity of the calcium-sensing receptor when the compound is not present.

In certain aspects, the method can further include analyzing two or more, three or more or four or more components in combination. In certain aspects, the two or more, three or more or four or more components can be from different classes of compounds, e.g., amino acids, disclosed salts, and/or other small chemical compounds. For example, and not by way of limitation, the method can include analyzing the effect of one or more taste modulator compositions on the biological activity and/or expression of a calcium-sensing receptor in the presence of one or more additional components. In certain aspects, the method for identifying compounds activity and/or expression of a calcium-sensing receptor comprises analyzing the effect of a taste modulator composition on the biological activity and/or expression of a calcium-sensing receptor in the presence of one or more salts of a disclosed taste modulator composition, e.g., a calcium salt.

In certain aspects, the method for assessing the effect of a disclosed taste modulator composition that modulates the activity and/or expression of a calcium-sensing receptor comprises determining whether a disclosed taste modulator composition modulates the receptor directly, for example, as an agonist. In certain aspects, the method comprises determining whether a disclosed taste modulator composition indirectly modulates the activity of the receptor (e.g., as an allosteric modulator), for example, by enhancing or decreasing the effect of other compounds on activating or inhibiting receptor activity.

In certain aspects, the method for assessing the effect of a disclosed taste modulator composition that modulates the activity and/or expression of a calcium-sensing receptor comprises expressing a calcium-sensing receptor in a cell line and measuring the biological activity of the receptor in the presence and/or absence of a taste modulator composition. The method can further comprise identifying taste modulator compositions that modulate the activity of the receptor by determining if there is a difference in receptor activation in the presence of a taste modulator composition compared to the activity of the receptor in the absence of the taste modulator composition. In certain aspects, the selectivity of the putative calcium-sensing receptor modulator can be evaluated by comparing its effects on other GPCRs or taste receptors, including, but not limited to umami, fatty acid, sweetener, umami, and bitterant receptors.

Activation of the receptor in the disclosed methods can be detected through the use of a labeling compound and/or agent. In certain aspects, the activity of the calcium-sensing receptor can be determined by the detection of secondary messengers such as, but not limited to, cAMP, cGMP, IP3, DAG or calcium. In certain aspects, the activity of the calcium-sensing receptor can be determined by the detection of the intracellular calcium levels. Monitoring can be by way of luminescence or fluorescence detection, such as by a calcium sensitive fluorescent dye. In certain aspects, the intracellular calcium levels can be determined using a cellular dye, e.g., a fluorescent calcium indicator such as Calcium 4. In certain aspects, the intracellular calcium levels can be determined by measuring the level of calcium binding to a calcium-binding protein, for example, calmodulin. Alternatively and/or additionally, activity of the calcium-sensing receptor can be determined by detection of the phosphorylation, transcript levels and/or protein levels of one or more downstream protein targets of the calcium-sensing receptor.

The cell line used in the disclosed methods can include any cell type that is capable of expressing a calcium-sensing receptor. Non-limiting examples of cells that can be used in the disclosed methods include HeLa cells, Chinese hamster ovary cells (CHO cells), African green monkey kidney cells (COS cells), Xenopus oocytes, HEK-293 cells and murine 3T3 fibroblasts. In certain aspects, the method can include expressing a calcium-sensing receptor in HEK-293 cells. In certain aspects, the method can include expressing a calcium-sensing receptor in COS cells. In certain aspects, the cells constitutively express the calcium-sensing receptor. In another aspect, expression of the CaSR by the cells is inducible.

In certain aspects, the cell expresses a calcium-binding photoprotein, wherein the photoprotein luminesces upon binding calcium. In certain aspects, the calcium binding photoprotein comprises the protein clytin. In certain aspects the clytin is a recombinant clytin. In certain aspects, the clytin comprises an isolated clytin, for example, a clytin isolated from Clytia gregarium. In certain aspects, the calcium-binding photoprotein comprises the protein aequorin, for example, a recombinant aequorin or an isolated aequorin, such as an aequorin isolated from Aequorea victoria. In certain aspects, the calcium-binding photoprotein comprises the protein obelin, for example, a recombinant obelin or an isolated obelin, such as an obelin isolated from Obelia longissima.

In certain aspects, expression of a calcium-sensing receptor in a cell can be performed by introducing a nucleic acid encoding a calcium-sensing receptor into the cell. For example, and not by way of limitation, a nucleic acid having the nucleotide sequence corresponding to a described CaSR, e.g., GenBank Accession No. NM_000388, or a fragment thereof, can be introduced into a cell. In certain aspects, the introduction of a nucleic acid into a cell can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. 217:599-618 (1993); Cohen et al., Meth. Enzymol. 217:618-644 (1993); Cline, Pharmac. Ther. 29:69-92 (1985), the disclosures of which are hereby incorporated by reference in their entireties) and can be used in accordance with the disclosed subject matter. In certain aspects, the technique can provide for stable transfer of nucleic acid to the cell, so that the nucleic acid is expressible by the cell and inheritable and expressible by its progeny. In certain aspects, the technique can provide for a transient transfer of the nucleic acid to the cell, so that the nucleic acid is expressible by the cell, wherein heritability and expressibility decrease in subsequent generations of the cell's progeny.

In certain aspects, the nucleic acid encoding a calcium-sensing receptor is comprised in a cloning vector, for example, a pcDNA3.1 vector or a pcDNA5 TO vector, that is introduced into the cell.

In certain aspects, the method can include assessing disclosed taste modulator compositions that bind to a calcium-sensing receptor. The method can comprise contacting a calcium-sensing receptor with a disclosed taste modulator composition and measuring binding between the compound and the calcium-sensing receptor. For example, and not by way of limitation, the methods can include providing an isolated or purified calcium-sensing receptor in a cell-free system, and contacting the receptor with a taste modulator composition in the cell-free system to determine if the taste modulator composition binds to the calcium-sensing receptor. In certain aspects, the method can comprise contacting a calcium-sensing receptor expressed on the surface of a cell with a candidate compound and detecting binding of the candidate compound to the calcium-sensing receptor. The binding can be measured directly, e.g., by using a labeled taste modulator composition, or can be measured indirectly. In certain aspects, the detection comprises detecting a physiological event in the cell caused by the binding of the compound to the calcium-sensing receptor, e.g., an increase in the intracellular calcium levels. For example, and not by way of limitation, detection can be performed by way of fluorescence detection, such as a calcium sensitive fluorescent dye, by detection of luminescence, or any other method of detection known in the art.

In certain non-limiting aspects, the in vitro assay comprises cells expressing a calcium-sensing receptor that is native to the cells. Examples of such cells expressing a native calcium-sensing receptor include, for example but not limited to, human taste cells or other suitable mammalian model (e.g., primary taste receptor cells). In certain aspects, the human taste cells expressing a calcium-sensing receptor are isolated from an ethically obtained human tissue, and then cultured in vitro. In certain aspects, the taste receptor cells can be immortalized, for example, such that the cells isolated from a human can be propagated in culture.

In certain aspects, expression of a calcium-sensing receptor in a cell can be induced through gene editing, for example, through use of the CRISPR gene editing system to incorporate a calcium-sensing receptor gene into the genome of a cell, or to edit or modify a calcium-sensing receptor gene native to the cell.

In certain aspects, the in vitro methods of assessing a disclosed taste modulator composition that binds to a calcium-sensing receptor comprises determining whether a taste modulator composition interacts with one or more amino acids of a calcium-sensing receptor interacting domain, as described herein.

In certain aspects, assessing a taste modulator composition identified as modulators of a calcium-sensing receptor can be further tested in other analytical methods including, but not limited to, in vivo assays, to confirm or quantitate their modulating activity.

In certain aspects, methods described herein can comprise determining whether a disclosed taste modulator composition is a kokumi taste enhancing compound, e.g., a calcium-sensing receptor agonist.

In certain aspects, the methods of assessing a disclosed taste modulator composition can comprise comparing the effect of a taste modulator composition to a calcium-sensing receptor agonist. For example, a taste modulator composition that increases the activity of the receptor compared to the activity of the receptor when contacted with a calcium-sensing receptor agonist can be selected as a calcium-sensing receptor modulating compound (e.g., as an agonist).

In certain aspects, the methods of assessing a disclosed taste modulator composition can comprise determining whether a taste modulator composition modulates the activity of the receptor when the receptor is contacted with an agonist, or whether the taste modulator composition can modulate the activity of a positive allosteric modulator (PAM). Taste modulator compositions that increase or decrease the effect of said agonist or PAM on the receptor can be selected as a calcium-sensing receptor modulating compound (e.g., as an allosteric modulator).

In certain aspects, the method can include predicting the three-dimensional structure (3D) of a calcium-sensing receptor and screening the predicted 3D structure with disclosed taste modulator compositions. The method can further include predicting whether the disclosed taste modulator compositions can interact with the binding site of the receptor by analyzing the potential interactions with the taste modulator composition and the amino acids of the receptor. The method can further include identifying a taste modulator composition that can bind to and/or modulate the biological activity of the calcium-sensing receptor by determining whether the 3D structure of the compound fits within the binding site of the 3D structure of the receptor.

In some aspects, the CaSR for use in the disclosed method can have the amino acid sequence corresponding to a described CaSR, e.g., GenBank Accession No. NM_000388, or a fragment or variant thereof. In certain aspects, the calcium-sensing receptor for use in the presently disclosed subject matter can include a receptor comprising an amino acid sequence having at least 85%, at least 90%, at least 91%, at least 92%, at least 93%, at least 94%, at least 95%, at least 96%, at least 97%, at least 98% or at least 99% identity to an amino acid sequence corresponding to a described CaSR, e.g., GenBank Accession No. NM_000388, or a fragment or variant thereof. In certain aspects, the calcium-sensing receptor for use in the disclosed method can have the nucleotide sequence corresponding to a described CaSR, e.g., GenBank Accession No. NM_000388, or a fragment or variant thereof. In certain aspects, the calcium-sensing receptor for use in the presently disclosed subject matter can include a receptor comprising a nucleotide sequence having at least 85%, at least 90%, at least 91%, at least 92%, at least 93%, at least 94%, at least 95%, at least 96%, at least 97%, at least 98% or at least 99% identity to a nucleic acid sequence corresponding to a described CaSR, e.g., GenBank Accession No. NM_000388, or a fragment or variant thereof.

In certain aspects, structural models of a calcium-sensing receptor can be built using crystal structures of other GPCRs as templates for homology modelling. For example, and not by way of limitation, structural models can be generated using the crystal structures of Group C GPCRs. In certain aspects, a structural model of a calcium-sensing receptor can be based on a known or a combination of known crystal structures of GPCRs. (See, e.g., Lee et al., Eur J Pharmacol. 2015 May 14. pii: S0014-2999(15)30012-1, which is incorporated by reference in its entirety herein). In certain aspects, a structural model of a calcium-sensing receptor can be generated based on the crystal structure of an mGluR protein. For example, and not by way of limitation, a structural model of the flytrap domain (VFT) of a calcium-sensing receptor can be generated based on the crystal structure having the protein data base (PDB) ID No. 1EWK. In certain aspects, a structural model of the 7 transmembrane domain (7TM) of a calcium-sensing receptor can be generated based on the crystal structures of mGluR proteins having PDB ID Nos. 4OR2 and 4OO9. FIG. 13 depict structural models of calcium-sensing receptors that can be used in the disclosed in silico methods. Any suitable modeling software known in the art can be used. In certain aspects, the Modeller software package can be used to generate the three-dimensional protein structure.

In some aspects, the in silico methods of assessing a disclosed taste modulator composition that binds to a CaSR comprises determining whether the taste modulator composition interacts with one or more amino acids of a calcium-sensing receptor interacting domain, as described herein.

Taste modulator compositions assessed by the disclosed in silico methods can be further assessed using the in vitro methods disclosed herein.

### Food and Beverage Products

In various aspects, the present disclosure, relates to beverage compositions comprising one or more taste modulator components. The taste modulator component improves key properties associated with the beverage composition, including overall taste response; mitigation of various flavor profile issues, in particular bitter taste or bitterness taste profile; improves desensitization/adaptation profile issues; and improves body/mouthfeel characteristics. In a further aspect, the disclosed taste modulator compositions comprise a first taste modulator component comprising a first salt having a first cation selected from Na⁺, K⁺, Ca²⁺, and Mg²⁺. The beverage compositions can optionally further comprise one or more additional taste modulator components, e.g., a second taste modulator component comprising a second salt having a second cation selected from Na⁺, K⁺, Ca²⁺, and Mg²⁺; a third taste modulator component comprising a third salt having a third cation selected from Na⁺, K⁺, Ca²⁺, and Mg²⁺; and a fourth taste modulator component comprising a fourth salt having a fourth cation selected from Na⁺, K⁺, Ca²⁺, and Mg²⁺. In some instances, the additional taste modulator components each comprise a different cation selected from Na⁺, K⁺, Ca²⁺, and Mg²⁺.

In various aspects, a disclosed beverage or food product comprises a first salt having a first cation independently selected from Na⁺, K⁺, Ca²⁺, and Mg²⁺; optionally a second salt having a second cation independently selected from Na⁺, K⁺, Ca²⁺, and Mg²⁺; optionally a third salt having a third cation independently selected from Na⁺, K⁺, Ca²⁺, and Mg²⁺; and optionally a fourth salt having a fourth cation independently selected from Na⁺, K⁺, Ca²⁺, and Mg²⁺; provided that first cation, second cation, third cation, and fourth cation are not the same.

In a further aspect, a disclosed beverage or food product comprises a first salt having a first cation and a first anion, such that the first cation is selected from Na⁺, K⁺, Ca²⁺, and Mg²⁺; and a first anion is selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₈O₄⁻²), hydrogen adipate C₆H₉O₄⁻¹), lactate (C₃H₅O₃⁻¹), tartrate (C₄H₄O₅⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof. In a specific aspect, the first anion comprises citrate (C₆H₆O₇⁻³), chloride (Cl⁻), phosphate (PO₄⁻³), carbonate (CO₃⁻²), and combinations thereof. Alternatively, in an aspect, the first anion comprises citrate (C₆H₆O₇⁻³) or the first anion comprises chloride (Cl⁻).

In a further aspect, a disclosed beverage or food product comprises a first salt having a first cation and a first anion; optionally a second salt having a second cation and a second anion; optionally a third salt having a third cation and a third anion; and optionally a fourth salt having a fourth cation and a fourth anion; the first cation is independently selected from Na⁺, K⁺, Ca²⁺, and Mg²⁺; the second cation is independently selected from Na⁺, K⁺, Ca²⁺, and Mg²⁺; the third cation is independently selected from Na⁺, K⁺, Ca²⁺, and Mg²⁺; and the fourth cation is independently selected from Na⁺, K⁺, Ca²⁺, and Mg²⁺; the first anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₃O₄⁻²), hydrogen adipate C₆H₉O₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; the second anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₈O₄⁻²), hydrogen adipate C₆H₉O₄⁻¹), lactate (C₃H₅O₃⁻¹), tartrate (C₄H₄O₅⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; the third anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₈O₄⁻²), hydrogen adipate C₆H₉O₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; and the fourth anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₈O₄⁻²), hydrogen adipate C₆H₉O₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof. In some instances, the first cation, the second cation, the third cation, and the fourth cation are not the same. In other instances, some or all of the first cation, the second cation, the third cation, and the fourth cation can be the same, provided that the first anion, the second anion, the third anion, and the fourth anion are not the same.

In a further aspect, a disclosed beverage or food product comprises a first salt having a first cation and a first anion; a second salt having a second cation and a second anion; optionally a third salt having a third cation and a third anion; and optionally a fourth salt having a fourth cation and a fourth anion; the first cation is independently selected from Na⁺, K⁺, Ca²⁺, and Mg²⁺; the second cation is independently selected from Na⁺, K⁺, Ca²⁺, and Mg²⁺; the third cation is independently selected from Na⁺, K⁺, Ca²⁺, and Mg²⁺; and the fourth cation is independently selected from Na⁺, K⁺, Ca²⁺, and Mg²⁺; the first anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₈O₄⁻²), hydrogen adipate C₆H₉O₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; the second anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₈O₄⁻²), hydrogen adipate C₆H₉O₄⁻¹), lactate (C₃H₅O₃⁻¹), tartrate (C₄H₄O₅⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; the third anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₆H₅O₄⁻²), hydrogen glutarate (C₅H₇O₄⁻¹), adipate C₆H₃O₄⁻²), hydrogen adipate C₆H₉O₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; and the fourth anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₃O₄⁻²), hydrogen adipate C₆H₉O₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof. In some instances, the first cation, the second cation, the third cation, and the fourth cation are not the same. In other instances, some or all of the first cation, the second cation, the third cation, and the fourth cation can be the same, provided that the first anion, the second anion, the third anion, and the fourth anion are not the same.

In a further aspect, a disclosed beverage or food product a first salt having a first cation and a first anion; a second salt having a second cation and a second anion; a third salt having a third cation and a third anion; and optionally a fourth salt having a fourth cation and a fourth anion; the first cation is independently selected from Na⁺, K⁺, Ca²⁺, and Mg²⁺; the second cation is independently selected from Na⁺, K⁺, Ca²⁺, and Mg²⁺; the third cation is independently selected from Na⁺, K⁺, Ca²⁺, and Mg²⁺; and the fourth cation is independently selected from Na⁺, K⁺, Ca²⁺, and Mg²⁺; the first anion is independently selected from gluconate (C₆H₁₁O₇⁻¹), citrate (C₆H₅O₇⁻³), hydrogen citrate (C₆H₆O₄⁻²), dihydrogen citrate (C₆H₇O₇⁻¹), malate (C₄H₆O₅²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₆HO₄⁻²), hydrogen glutarate (C₅H₇O₄⁻¹), adipate C₆H₈O₄⁻²), hydrogen adipate C₆H₉O₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; the second anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₃O₄⁻²), hydrogen adipate C₆H₉O₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; the third anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₇⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₃O₄⁻²), hydrogen adipate C₆H₉O₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; and the fourth anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₆H₆O₄⁻²), hydrogen glutarate (C₅H₇O₄⁻¹), adipate C₆H₈O₄⁻²), hydrogen adipate C₆HO₄⁻¹), lactate (C₃H₅O₃⁻¹), tartrate (C₄H₄O₅⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof. In some instances, the first cation, the second cation, the third cation, and the fourth cation are not the same. In other instances, some or all of the first cation, the second cation, the third cation, and the fourth cation can be the same, provided that the first anion, the second anion, the third anion, and the fourth anion are not the same.

In a further aspect, a disclosed beverage or food product comprises a first salt having a first cation and a first anion; a second salt having a second cation and a second anion; a third salt having a third cation and a third anion; the first cation is independently selected from Na⁺, K⁺, Ca²⁺, and Mg²⁺; the second cation is independently selected from Na⁺, K⁺, Ca²⁺, and Mg²⁺; the third cation is independently selected from Na⁺, K⁺, Ca²⁺, and Mg²⁺; the first anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₈O₄⁻²), hydrogen adipate C₆HO₄⁻¹), lactate (C₃H₅O₃⁻¹), tartrate (C₄H₄O₆⁻²), bitartrate (C₄H₅O₆₀⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; the second anion is independently selected from gluconate (C₆H₁₁O₇¹−), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₆H₆O₄⁻²), hydrogen glutarate (C₅H₇O₄⁻¹), adipate C₆H₃O₄⁻²), hydrogen adipate C₆HO₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; and the third anion is independently selected from gluconate (CH₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₃O₄⁻²), hydrogen adipate C₆H₉O₄⁻¹), lactate (C₃H₅O₃⁻¹), tartrate (C₄H₄O₅⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof. In some instances, the first cation, the second cation, the third cation, and the fourth cation are not the same. In other instances, some or all of the first cation, the second cation, the third cation, and the fourth cation can be the same, provided that the first anion, the second anion, the third anion, and the fourth anion are not the same.

In a further aspect, a disclosed beverage or food product comprises a first salt having a first cation and a first anion; a second salt having a second cation and a second anion; a third salt having a third cation and a third anion; the first cation is K⁺; the second cation is Mg²⁺; the third cation is Ca²⁺; the first anion is independently selected from gluconate (C₆H₁₁O₇⁻¹), citrate (C₆H₅O₇⁻⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₈O₄⁻²), hydrogen adipate C₆H₉O₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; the second anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₈O₄⁻²), hydrogen adipate C₆H₉O₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof; and the third anion is independently selected from gluconate (C₆H₁₁O₇⁻¹), citrate (C₆H₅O₇⁻³), hydrogen citrate (C₆H₆O₇⁻²), dihydrogen citrate (C₆H₇O₇⁻¹), malate (C₄H₆O₅⁻²), hydrogen malate (C₄H₇O₅⁻¹), maleate (C₄H₂O₄⁻²), hydrogen maleate (C₄H₃O₄⁻¹), fumarate (C₄H₂O₄⁻²), hydrogen fumarate (C₄H₃O₄⁻¹), succinate (C₄H₄O₄⁻²), hydrogen succinate (C₄H₅O₄⁻¹), glutarate (C₅H₆O₄⁻²), hydrogen glutarate (C₅H₇O₄⁻¹), adipate C₆H₈O₄⁻²), hydrogen adipate C₆H₉O₄⁻¹), lactate (C₃H₅O₃⁻¹), tartrate (C₄H₄O₆⁻²), bitartrate (C₄H₅O₆⁻¹), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻¹), nitrate (NO₃⁻), carbonate (CO₃⁻²), bicarbonate (HCO₃⁻), glycerate (C₃H₅O₄⁻¹), glycolate (C₂H₃O₃⁻¹), or combinations thereof. In some instances, the first cation, the second cation, the third cation, and the fourth cation are not the same. In other instances, some or all of the first cation, the second cation, the third cation, and the fourth cation can be the same, provided that the first anion, the second anion, the third anion, and the fourth anion are not the same.

In a further aspect, a disclosed beverage or food product comprises a first salt having a first cation and a first anion; a second salt having a second cation and a second anion; a third salt having a third cation and a third anion; the first cation is K⁺; the second cation is Mg²⁺; the third cation is Ca²⁺; the first anion is independently selected from citrate (C₆H₅O₇⁻³) or conjugate acid form thereof, chloride (Cl⁻), sulfate (SO₄⁻²), or bisulfate (HSO₄⁻¹), or combinations thereof; the second anion is independently selected from citrate (C₆H₅O₇⁻³) or conjugate acid form thereof, chloride (Cl⁻), sulfate (SO₄⁻²), or bisulfate (HSO₄⁻¹), or combinations thereof; and the third anion is independently selected from citrate (C₆H₅O₇⁻³) or conjugate acid form thereof, chloride (Cl⁻), sulfate (SO₄⁻²), or bisulfate (HSO₄⁻¹), or combinations thereof. In some instances, the first cation, the second cation, the third cation, and the fourth cation are not the same. In other instances, some or all of the first cation, the second cation, the third cation, and the fourth cation can be the same, provided that the first anion, the second anion, the third anion, and the fourth anion are not the same.

In various aspects, a disclosed beverage or food product comprises a first taste modulator component comprising a first salt having a first cation selected from Na⁺, K⁺, Ca²⁺, and Mg²⁺ and a first anion; and a second modulator component comprising a second salt having a second cation selected from Na⁺, K⁺, Ca²⁺, and Mg²⁺ and a second anion; such that the first taste modulator component is present at a concentration of from about 0 mM to about 25 mM if the first cation is Na⁺ or K⁺, or at a concentration of from about 0 mM to about 10 mM if the first cation is Ca²⁺ or Mg²⁺; and such that the second taste modulator component is present at a concentration of from about 0 mM to about 25 mM if the second cation is Na⁺ or K⁺, or at a concentration of from about 0 mM to about 10 mM if the second cation is Ca²⁺ or Mg².

In various aspects, a disclosed beverage or food product comprises a first taste modulator component comprising a first salt having a first cation selected from Na⁺, K⁺, Ca²⁺, and Mg²⁺ and a first anion; and a second modulator component comprising a second salt having a second cation selected from Na⁺, K⁺, Ca²⁺, and Mg²⁺ and a second anion; such that the first taste modulator component is present at a concentration of from about 0 mM to about 25 mM if the first cation is Na⁺ or K⁺, or at a concentration of from about 0 mM to about 5 mM if the first cation is Ca²⁺ or Mg²⁺; and such that the second taste modulator component is present at a concentration of from about 0 mM to about 25 mM if the second cation is Na⁺ or K⁺, or at a concentration of from about 0 mM to about 10 mM if the second cation is Ca²⁺ or Mg²⁺ or K⁺, or at a concentration of from about 0 mM to about 10 mM if the third cation is Ca²⁺ or Mg²⁺.

In various aspects, a disclosed beverage or food product comprises a first taste modulator component comprising a first salt having a first cation selected from Na⁺, K⁺, Ca²⁺, and Mg²⁺ and a first anion; and a second modulator component comprising a second salt having a second cation selected from Na⁺, K⁺, Ca²⁺, and Mg²⁺ and a second anion; such that the first taste modulator component is present at a concentration of from about 0 mM to about 10 mM if the first cation is Na⁺ or K⁺, or at a concentration of from about 0 mM to about 5 mM if the first cation is Ca²⁺ or Mg²⁺; and such that the second taste modulator component is present at a concentration of from about 0 mM to about 10 mM if the second cation is Na⁺ or K⁺, or at a concentration of from about 0 mM to about 5 mM if the second cation is Ca²⁺ or Mg²⁺.

In various aspects, a disclosed beverage or food product comprises a first taste modulator component comprising a first salt having a first cation selected from Na⁺, K⁺, Ca²⁺, and Mg²⁺ and a first anion; a second modulator component comprising a second salt having a second cation selected from Na⁺, K⁺, Ca²⁺, and Mg²⁺ and a second anion; such that the first taste modulator component is present at a concentration of from about 0.1 mM to about 25 mM if the first cation is Na⁺ or K⁺, or at a concentration of from about 0.1 mM to about 10 mM if the first cation is Ca²⁺ or Mg²⁺; and such that the second taste modulator component is present at a concentration of from about 0.1 mM to about 25 mM if the second cation is Na⁺ or K⁺, or at a concentration of from about 0.1 mM to about 10 mM if the second cation is Ca²⁺ or Mg²⁺ or Mg²⁺.

In various aspects, a disclosed beverage or food product comprises a first taste modulator component comprising a first salt having a first cation selected from Na⁺, K⁺, Ca²⁺, and Mg²⁺ and a first anion; a second modulator component comprising a second salt having a second cation selected from Na⁺, K⁺, Ca²⁺, and Mg²⁺ and a second anion; such that the first taste modulator component is present at a concentration of from about 0.1 mM to about 25 mM if the first cation is Na⁺ or K⁺, or at a concentration of from about 0.1 mM to about 5 mM if the first cation is Ca²⁺ or Mg²⁺; and such that the second taste modulator component is present at a concentration of from about 0.1 mM to about 25 mM if the second cation is Na⁺ or K⁺, or at a concentration of from about 0.1 mM to about 10 mM if the second cation is Ca²⁺ or Mg²⁺.

In various aspects, a disclosed beverage or food product comprises a first taste modulator component comprising a first salt having a first cation selected from Na⁺, K⁺, Ca²⁺, and Mg²⁺ and a first anion; a second modulator component comprising a second salt having a second cation selected from Na⁺, K⁺, Ca²⁺, and Mg²⁺ and a second anion; such that the first taste modulator component is present at a concentration of from about 0.1 mM to about 10 mM if the first cation is Na⁺ or K⁺, or at a concentration of from about 0.1 mM to about 5 mM if the first cation is Ca²⁺ or Mg²⁺; and such that the second taste modulator component is present at a concentration of from about 0.1 mM to about 10 mM if the second cation is Na⁺ or K⁺, or at a concentration of from about 0.1 mM to about 5 mM if the second cation is Ca²⁺ or Mg²⁺.

In various aspects, a disclosed beverage or food product comprises a first taste modulator component comprising a first salt having a first cation selected from Na⁺, K⁺, Ca²⁺, and Mg²⁺ and a first anion; a second modulator component comprising a second salt having a second cation selected from Na⁺, K⁺, Ca²⁺, and Mg²⁺ and a second anion; such that the first taste modulator component is present at a concentration of from about 1 mM to about 25 mM if the first cation is Na⁺ or K⁺, or at a concentration of from about 1 mM to about 10 mM if the first cation is Ca²⁺ or Mg²⁺; and such that the second taste modulator component is present at a concentration of from about 1 mM to about 25 mM if the second cation is Na⁺ or K⁺, or at a concentration of from about 1 mM to about 10 mM if the second cation is Ca²⁺ or Mg²⁺.

In various aspects, a disclosed beverage or food product comprises a first taste modulator component comprising a first salt having a first cation selected from Na⁺, K⁺, Ca²⁺, and Mg²⁺ and a first anion; a second modulator component comprising a second salt having a second cation selected from Na⁺, K⁺, Ca²⁺, and Mg²⁺ and a second anion; such that the first taste modulator component is present at a concentration of from about 1 mM to about 25 mM if the first cation is Na⁺ or K⁺, or at a concentration of from about 1 mM to about 5 mM if the first cation is Ca²⁺ or Mg²⁺; and such that the second taste modulator component is present at a concentration of from about 1 mM to about 25 mM if the second cation is Na⁺ or K⁺, or at a concentration of from about 1 mM to about 10 mM if the second cation is Ca²⁺ or Mg²⁺.

In various aspects, a disclosed beverage or food product comprises a first taste modulator component comprising a first salt having a first cation selected from Na⁺, K⁺, Ca²⁺, and Mg²⁺ and a first anion; a second modulator component comprising a second salt having a second cation selected from Na⁺, K⁺, Ca²⁺, and Mg²⁺ and a second anion; such that the first taste modulator component is present at a concentration of from about 1 mM to about 10 mM if the first cation is Na⁺ or K⁺, or at a concentration of from about 1 mM to about 5 mM if the first cation is Ca²⁺ or Mg²⁺; and such that the second taste modulator component is present at a concentration of from about 1 mM to about 10 mM if the second cation is Na⁺ or K⁺, or at a concentration of from about 1 mM to about 5 mM if the second cation is Ca²⁺ or Mg²⁺.

In various aspects, a disclosed beverage or food product comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0 mM to about 25 mM if the first cation is Na⁺ or K⁺, or at a concentration of from about 0 mM to about 10 mM if the first cation is Ca²⁺ or Mg²⁺; such that the second taste modulator component is present at a concentration of from about 0 mM to about 25 mM if the second cation is Na⁺ or K⁺, or at a concentration of from about 0 mM to about 10 mM if the second cation is Ca²⁺ or Mg²⁺; and such that the third taste modulator component is present at a concentration of from about 0 mM to about 25 mM if the third cation is Na⁺ or K⁺, or at a concentration of from about 0 mM to about 10 mM if the third cation is Ca²⁺ or Mg²⁺.

In various aspects, a disclosed beverage or food product comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0 mM to about 25 mM if the first cation is Na⁺ or K⁺, or at a concentration of from about 0 mM to about 5 mM if the first cation is Ca²⁺ or Mg²⁺; such that the second taste modulator component is present at a concentration of from about 0 mM to about 25 mM if the second cation is Na⁺ or K⁺, or at a concentration of from about 0 mM to about 10 mM if the second cation is Ca²⁺ or Mg²⁺; and such that the third taste modulator component is present at a concentration of from about 0 mM to about 25 mM if the third cation is Na⁺ or K⁺, or at a concentration of from about 0 mM to about 10 mM if the third cation is Ca²⁺ or Mg²⁺.

In various aspects, a disclosed beverage or food product comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0 mM to about 10 mM if the first cation is Na⁺ or K⁺, or at a concentration of from about 0 mM to about 5 mM if the first cation is Ca²⁺ or Mg²⁺; such that the second taste modulator component is present at a concentration of from about 0 mM to about 10 mM if the second cation is Na⁺ or K⁺, or at a concentration of from about 0 mM to about 5 mM if the second cation is Ca²⁺ or Mg²⁺; and such that the third taste modulator component is present at a concentration of from about 0 mM to about 10 mM if the third cation is Na⁺ or K⁺, or at a concentration of from about 0 mM to about 5 mM if the third cation is Ca²⁺ or Mg²⁺.

In various aspects, a disclosed beverage or food product comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0.1 mM to about 25 mM if the first cation is Na⁺ or K⁺, or at a concentration of from about 0.1 mM to about 10 mM if the first cation is Ca²⁺ or Mg²⁺; such that the second taste modulator component is present at a concentration of from about 0.1 mM to about 25 mM if the second cation is Na⁺ or K⁺, or at a concentration of from about 0.1 mM to about 10 mM if the second cation is Ca²⁺ or Mg²⁺; and such that the third taste modulator component is present at a concentration of from about 0.1 mM to about 25 mM if the third cation is Na⁺ or K⁺, or at a concentration of from about 0.1 mM to about 10 mM if the third cation is Ca²⁺ or Mg²⁺.

In various aspects, a disclosed beverage or food product comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0.1 mM to about 25 mM if the first cation is Na⁺ or K⁺, or at a concentration of from about 0.1 mM to about 5 mM if the first cation is Ca²⁺ or Mg²⁺; such that the second taste modulator component is present at a concentration of from about 0.1 mM to about 25 mM if the second cation is Na⁺ or K⁺, or at a concentration of from about 0.1 mM to about 10 mM if the second cation is Ca²⁺ or Mg²⁺; and such that the third taste modulator component is present at a concentration of from about 0.1 mM to about 25 mM if the third cation is Na⁺ or K⁺, or at a concentration of from about 0.1 mM to about 10 mM if the third cation is Ca²⁺ or Mg²⁺.

In various aspects, a disclosed beverage or food product comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0.1 mM to about 10 mM if the first cation is Na⁺ or K⁺, or at a concentration of from about 0.1 mM to about 5 mM if the first cation is Ca²⁺ or Mg²⁺; such that the second taste modulator component is present at a concentration of from about 0.1 mM to about 10 mM if the second cation is Na⁺ or K⁺, or at a concentration of from about 0.1 mM to about 5 mM if the second cation is Ca²⁺ or Mg²⁺; and such that the third taste modulator component is present at a concentration of from about 0.1 mM to about 10 mM if the third cation is Na⁺ or K⁺, or at a concentration of from about 0.1 mM to about 5 mM if the third cation is Ca²⁺ or Mg²⁺.

In various aspects, a disclosed beverage or food product comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 1 mM to about 25 mM if the first cation is Na⁺ or K⁺, or at a concentration of from about 1 mM to about 10 mM if the first cation is Ca²⁺ or Mg²⁺; such that the second taste modulator component is present at a concentration of from about 1 mM to about 25 mM if the second cation is Na⁺ or K⁺, or at a concentration of from about 1 mM to about 10 mM if the second cation is Ca²⁺ or Mg²⁺; and such that the third taste modulator component is present at a concentration of from about 1 mM to about 25 mM if the third cation is Na⁺ or K⁺, or at a concentration of from about 1 mM to about 10 mM if the third cation is Ca²⁺ or Mg²⁺.

In various aspects, a disclosed beverage or food product comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 1 mM to about 25 mM if the first cation is Na⁺ or K⁺, or at a concentration of from about 1 mM to about 5 mM if the first cation is Ca²⁺ or Mg²⁺; such that the second taste modulator component is present at a concentration of from about 1 mM to about 25 mM if the second cation is Na⁺ or K⁺, or at a concentration of from about 1 mM to about 10 mM if the second cation is Ca²⁺ or Mg²⁺; and such that the third taste modulator component is present at a concentration of from about 1 mM to about 25 mM if the third cation is Na⁺ or K⁺, or at a concentration of from about 1 mM to about 10 mM if the third cation is Ca²⁺ or Mg²⁺.

In various aspects, a disclosed beverage or food product comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 1 mM to about 10 mM if the first cation is Na⁺ or K⁺, or at a concentration of from about 1 mM to about 5 mM if the first cation is Ca²⁺ or Mg²⁺; such that the second taste modulator component is present at a concentration of from about 1 mM to about 10 mM if the second cation is Na⁺ or K⁺, or at a concentration of from about 1 mM to about 5 mM if the second cation is Ca²⁺ or Mg²⁺; and such that the third taste modulator component is present at a concentration of from about 1 mM to about 10 mM if the third cation is Na⁺ or K⁺, or at a concentration of from about 1 mM to about 5 mM if the third cation is Ca²⁺ or Mg²⁺.

In various aspects, a disclosed beverage or food product comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0 mM to about 25 mM; and such that each of the second taste modulator component and the third modulator component are independently present at a concentration of from about 0 mM to about 10 mM.

In various aspects, a disclosed beverage or food product comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0 mM to about 25 mM; and such that each of the second taste modulator component and the third modulator component are independently present at a concentration of from about 0 mM to about 5 mM.

In various aspects, a disclosed beverage or food product comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0 mM to about 10 mM; and such that each of the second taste modulator component and the third modulator component are independently present at a concentration of from about 0 mM to about 5 mM.

In various aspects, a disclosed beverage or food product comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0.1 mM to about 25 mM; and such that each of the second taste modulator component and the third modulator component are independently present at a concentration of from about 0.1 mM to about 10 mM.

In various aspects, a disclosed beverage or food product comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0.1 mM to about 25 mM; and such that each of the second taste modulator component and the third modulator component are independently present at a concentration of from about 0.1 mM to about 5 mM.

In various aspects, a disclosed beverage or food product comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0.1 mM to about 10 mM; and such that each of the second taste modulator component and the third modulator component are independently present at a concentration of from about 0.1 mM to about 5 mM.

In various aspects, a disclosed beverage or food product comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 1 mM to about 25 mM; and such that each of the second taste modulator component and the third modulator component are independently present at a concentration of from about 1 mM to about 10 mM.

In various aspects, a disclosed beverage or food product comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 1 mM to about 25 mM; and such that each of the second taste modulator component and the third modulator component are independently present at a concentration of from about 1 mM to about 5 mM.

In various aspects, a disclosed beverage or food product comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 1 mM to about 10 mM; and such that each of the second taste modulator component and the third modulator component are independently present at a concentration of from about 1 mM to about 5 mM.

In various aspects, a disclosed beverage or food product comprises a first taste modulator component comprising a first salt having a first cation selected from Na⁺, K⁺, Ca²⁺, and Mg²⁺ and a first anion selected from citrate (C₆H₅O₇⁻³), chloride (Cl⁻), phosphate (PO₄⁻³), carbonate (CO₃⁻²), sulfate (SO₄⁻²), and combinations thereof; a second modulator component comprising a second salt having a second cation selected from Na⁺, K⁺, Ca²⁺, and Mg²⁺ and a second anion selected from citrate (C₆H₅O₇⁻³), chloride (Cl⁻), phosphate (PO₄⁻³), carbonate (CO₃⁻²), sulfate (SO₄⁻²), and combinations thereof; and a third modulator component comprising a third salt having a third cation selected from Na⁺, K⁺, Ca²⁺, and Mg²⁺ and a third anion selected from citrate (CH₅O₇⁻³), chloride (Cl⁻), phosphate (PO₄⁻³), carbonate (CO₃⁻²), sulfate (SO₄⁻²), and combinations thereof; such that the first taste modulator component is present at a concentration of from about 0 mM to about 10 mM; and such that each of the second taste modulator component and the third modulator component are independently present at a concentration of from about 0 mM to about 5 mM.

In various aspects, a disclosed beverage or food product comprises a first taste modulator component comprising a first salt having a first cation selected from Na⁺, K⁺, Ca²⁺, and Mg²⁺; a second modulator component comprising a second salt having a second cation selected from Na⁺, K⁺, Ca²⁺, and Mg²⁺; and a third modulator component comprising a third salt having a third cation selected from Na⁺, K⁺, Ca²⁺, and Mg²⁺; such that the first taste modulator component is present at a concentration of from about 0.1 mM to about 10 mM; and such that each of the second taste modulator component and the third modulator component are independently present at a concentration of from about 0.1 mM to about 5 mM.

In various aspects, a disclosed beverage or food product comprises a first taste modulator component comprising a first salt having a first cation selected from Na⁺, K⁺, Ca²⁺, and Mg²⁺ and a first anion selected from citrate (C₆H₅O₇⁻³), chloride (Cl⁻), phosphate (PO₄⁻³), carbonate (CO₃⁻²), sulfate (SO₄⁻²), and combinations thereof; a second modulator component comprising a second salt having a second cation selected from Na⁺, K⁺, Ca²⁺, and Mg²⁺ and a second anion selected from citrate (C₆H₅O₇⁻³), chloride (Cl⁻), phosphate (PO₄⁻³), carbonate (CO₃⁻²), sulfate (SO₄⁻²), and combinations thereof; and a third modulator component comprising a third salt having a third cation selected from Na⁺, K⁺, Ca²⁺, and Mg²⁺ and a third anion selected from citrate (C₆H₅O₇⁻³), chloride (Cl⁻), phosphate (PO₄⁻³), carbonate (CO₃⁻²), sulfate (SO₄⁻²), and combinations thereof; such that the first taste modulator component is present at a concentration of from about 0.1 mM to about 10 mM; and such that each of the second taste modulator component and the third modulator component are independently present at a concentration of from about 0.1 mM to about 5 mM.

In various aspects, a disclosed beverage or food product comprises a first taste modulator component comprising a first salt having a first cation comprising K⁺; a second modulator component comprising a second salt having a second cation comprising Mg²⁺; and a third modulator component comprising a third salt having a third cation comprising Ca²⁺; such that the first taste modulator component is present at a concentration of from about 0 mM to about 10 mM; and such that each of the second taste modulator component and the third modulator component are independently present at a concentration of from about 0 mM to about 5 mM.

In various aspects, a disclosed beverage or food product comprises a first taste modulator component comprising a first salt having a first cation comprising K⁺ and a first anion selected from citrate (C₆H₅O₇⁻³), chloride (Cl⁻), phosphate (PO₄⁻³), carbonate (CO₃⁻²), sulfate (SO₄⁻²), and combinations thereof; a second modulator component comprising a second salt having a second cation comprising Mg²⁺ and a second anion selected from citrate (C₆H₅O₇⁻³), chloride (Cl⁻), phosphate (PO₄⁻³), carbonate (CO₃⁻²), sulfate (SO₄⁻²), and combinations thereof; and a third modulator component comprising a third salt having a third cation comprising Ca²⁺ and a third anion selected from citrate (C₆H₅O₇⁻³), chloride (Cl⁻), phosphate (PO₄⁻³), carbonate (CO₃⁻²), sulfate (SO₄⁻²), and combinations thereof; such that the first taste modulator component is present at a concentration of from about 0 mM to about 10 mM; and such that each of the second taste modulator component and the third modulator component are independently present at a concentration of from about 0 mM to about 5 mM.

In various aspects, a disclosed beverage or food product comprises a first taste modulator component comprising a first salt having a first cation comprising K⁺; a second modulator component comprising a second salt having a second cation comprising Mg²⁺; and a third modulator component comprising a third salt having a third cation comprising Ca²⁺; such that the first taste modulator component is present at a concentration of from about 0.1 mM to about 10 mM; and such that each of the second taste modulator component and the third modulator component are independently present at a concentration of from about 0.1 mM to about 5 mM.

In various aspects, a disclosed beverage or food product comprises a first taste modulator component comprising a first salt having a first cation comprising K⁺ and a first anion selected from citrate (C₆H₅O₇⁻³), chloride (Cl⁻), phosphate (PO₄⁻³), carbonate (CO₃⁻²), sulfate (SO₄⁻²), and combinations thereof; a second modulator component comprising a second salt having a second cation comprising Mg²⁺ and a second anion selected from citrate (C₆H₅O₇⁻³), chloride (Cl⁻), phosphate (PO₄⁻³), carbonate (CO₃⁻²), sulfate (SO₄⁻²), and combinations thereof; and a third modulator component comprising a third salt having a third cation comprising Ca²⁺ and a third anion selected from citrate (C₆H₅O₇⁻³), chloride (Cl⁻), phosphate (PO₄⁻³), carbonate (CO₃⁻²), sulfate (SO₄⁻²), and combinations thereof; such that the first taste modulator component is present at a concentration of from about 0.1 mM to about 10 mM; and such that each of the second taste modulator component and the third modulator component are independently present at a concentration of from about 0.1 mM to about 5 mM.

In various aspects, the total concentration of the first modulator component, the second modulator component, the third modulator component, and the fourth modulator component taken together can be from about 0.1 mM to about 30 mM. For example, each of the first modulator component, the second modulator component, the third modulator component, and the fourth modulator component are independently present a concentration of from about 0 mM to about 10 mM, provided that the sum of concentrations of the first modulator component, the second modulator component, the third modulator component, and the fourth modulator component is less than about 30 mM. In a further aspect, the total concentration of the first modulator component, the second modulator component, the third modulator component, and the fourth modulator component taken together can be from about 0.1 mM to about 30 mM, about 0.2 mM to about 30 mM, about 0.3 mM to about 30 mM, about 0.4 mM to about 30 mM, about 0.5 mM to about 30 mM, about 0.6 mM to about 30 mM, about 0.7 mM to about 30 mM, about 0.8 mM to about 30 mM, about 0.9 mM to about 30 mM, about 1.0 mM to about 30 mM, about 0.1 mM to about 25 mM, about 0.2 mM to about 25 mM, about 0.3 mM to about 25 mM, about 0.4 mM to about 25 mM, about 0.5 mM to about 25 mM, about 0.6 mM to about 25 mM, about 0.7 mM to about 25 mM, about 0.8 mM to about 25 mM, about 0.9 mM to about 25 mM, about 1.0 mM to about 25 mM, about 0.1 mM to about 20 mM, about 0.2 mM to about 20 mM, about 0.3 mM to about 20 mM, about 0.4 mM to about 20 mM, about 0.5 mM to about 20 mM, about 0.6 mM to about 20 mM, about 0.7 mM to about 20 mM, about 0.8 mM to about 20 mM, about 0.9 mM to about 20 mM, about 1.0 mM to about 20 mM, about 0.1 mM to about 15 mM, about 0.2 mM to about 15 mM, about 0.3 mM to about 15 mM, about 0.4 mM to about 15 mM, about 0.5 mM to about 15 mM, about 0.6 mM to about 15 mM, about 0.7 mM to about 15 mM, about 0.8 mM to about 15 mM, about 0.9 mM to about 15 mM, about 1.0 mM to about 15 mM, about 0.1 mM to about 10 mM, about 0.2 mM to about 10 mM, about 0.3 mM to about 10 mM, about 0.4 mM to about 10 mM, about 0.5 mM to about 10 mM, about 0.6 mM to about 10 mM, about 0.7 mM to about 10 mM, about 0.8 mM to about 10 mM, about 0.9 mM to about 10 mM, about 1.0 mM to about 10 mM; or a concentration or set of concentrations within the foregoing concentration ranges; or a sub-range of any of the foregoing concentration ranges.

In a further aspect, the concentration of the first modulator component can be from about 0.1 mM to about 30 mM, about 0.2 mM to about 30 mM, about 0.3 mM to about 30 mM, about 0.4 mM to about 30 mM, about 0.5 mM to about 30 mM, about 0.6 mM to about 30 mM, about 0.7 mM to about 30 mM, about 0.8 mM to about 30 mM, about 0.9 mM to about 30 mM, about 1.0 mM to about 30 mM, about 0.1 mM to about 25 mM, about 0.2 mM to about 25 mM, about 0.3 mM to about 25 mM, about 0.4 mM to about 25 mM, about 0.5 mM to about 25 mM, about 0.6 mM to about 25 mM, about 0.7 mM to about 25 mM, about 0.8 mM to about 25 mM, about 0.9 mM to about 25 mM, about 1.0 mM to about 25 mM, about 0.1 mM to about 20 mM, about 0.2 mM to about 20 mM, about 0.3 mM to about 20 mM, about 0.4 mM to about 20 mM, about 0.5 mM to about 20 mM, about 0.6 mM to about 20 mM, about 0.7 mM to about 20 mM, about 0.8 mM to about 20 mM, about 0.9 mM to about 20 mM, about 1.0 mM to about 20 mM, about 0.1 mM to about 15 mM, about 0.2 mM to about 15 mM, about 0.3 mM to about 15 mM, about 0.4 mM to about 15 mM, about 0.5 mM to about 15 mM, about 0.6 mM to about 15 mM, about 0.7 mM to about 15 mM, about 0.8 mM to about 15 mM, about 0.9 mM to about 15 mM, about 1.0 mM to about 15 mM, about 0.1 mM to about 10 mM, about 0.2 mM to about 10 mM, about 0.3 mM to about 10 mM, about 0.4 mM to about 10 mM, about 0.5 mM to about 10 mM, about 0.6 mM to about 10 mM, about 0.7 mM to about 10 mM, about 0.8 mM to about 10 mM, about 0.9 mM to about 10 mM, about 1.0 mM to about 10 mM, about 0.1 mM to about 9 mM, about 0.2 mM to about 9 mM, about 0.3 mM to about 9 mM, about 0.4 mM to about 9 mM, about 0.5 mM to about 9 mM, about 0.6 mM to about 9 mM, about 0.7 mM to about 9 mM, about 0.8 mM to about 9 mM, about 0.9 mM to about 9 mM, about 1.0 mM to about 9 mM, about 0.1 mM to about 8 mM, about 0.2 mM to about 8 mM, about 0.3 mM to about 8 mM, about 0.4 mM to about 8 mM, about 0.5 mM to about 8 mM, about 0.6 mM to about 8 mM, about 0.7 mM to about 8 mM, about 0.8 mM to about 8 mM, about 0.9 mM to about 8 mM, about 1.0 mM to about 8 mM, about 0.1 mM to about 7 mM, about 0.2 mM to about 7 mM, about 0.3 mM to about 7 mM, about 0.4 mM to about 7 mM, about 0.5 mM to about 7 mM, about 0.6 mM to about 7 mM, about 0.7 mM to about 7 mM, about 0.8 mM to about 7 mM, about 0.9 mM to about 7 mM, about 1.0 mM to about 7 mM, about 0.1 mM to about 6 mM, about 0.2 mM to about 6 mM, about 0.3 mM to about 6 mM, about 0.4 mM to about 6 mM, about 0.5 mM to about 6 mM, about 0.6 mM to about 6 mM, about 0.7 mM to about 6 mM, about 0.8 mM to about 6 mM, about 0.9 mM to about 6 mM, about 1.0 mM to about 6 mM, about 0.1 mM to about 5 mM, about 0.2 mM to about 5 mM, about 0.3 mM to about 5 mM, about 0.4 mM to about 5 mM, about 0.5 mM to about 5 mM, about 0.6 mM to about 5 mM, about 0.7 mM to about 5 mM, about 0.8 mM to about 5 mM, about 0.9 mM to about 5 mM, about 1.0 mM to about 5 mM, about 0.1 mM to about 4 mM, about 0.2 mM to about 4 mM, about 0.3 mM to about 4 mM, about 0.4 mM to about 4 mM, about 0.5 mM to about 4 mM, about 0.6 mM to about 4 mM, about 0.7 mM to about 4 mM, about 0.8 mM to about 4 mM, about 0.9 mM to about 4 mM, about 1.0 mM to about 4 mM, about 0.1 mM to about 3 mM, about 0.2 mM to about 3 mM, about 0.3 mM to about 3 mM, about 0.4 mM to about 3 mM, about 0.5 mM to about 3 mM, about 0.6 mM to about 3 mM, about 0.7 mM to about 3 mM, about 0.8 mM to about 3 mM, about 0.9 mM to about 3 mM, about 1.0 mM to about 3 mM; or a concentration or set of concentrations within the foregoing concentration ranges; or a sub-range of any of the foregoing concentration ranges.

In a further aspect, the concentration of the second modulator component can be from about 0.1 mM to about 30 mM, about 0.2 mM to about 30 mM, about 0.3 mM to about 30 mM, about 0.4 mM to about 30 mM, about 0.5 mM to about 30 mM, about 0.6 mM to about 30 mM, about 0.7 mM to about 30 mM, about 0.8 mM to about 30 mM, about 0.9 mM to about 30 mM, about 1.0 mM to about 30 mM, about 0.1 mM to about 25 mM, about 0.2 mM to about 25 mM, about 0.3 mM to about 25 mM, about 0.4 mM to about 25 mM, about 0.5 mM to about 25 mM, about 0.6 mM to about 25 mM, about 0.7 mM to about 25 mM, about 0.8 mM to about 25 mM, about 0.9 mM to about 25 mM, about 1.0 mM to about 25 mM, about 0.1 mM to about 20 mM, about 0.2 mM to about 20 mM, about 0.3 mM to about 20 mM, about 0.4 mM to about 20 mM, about 0.5 mM to about 20 mM, about 0.6 mM to about 20 mM, about 0.7 mM to about 20 mM, about 0.8 mM to about 20 mM, about 0.9 mM to about 20 mM, about 1.0 mM to about 20 mM, about 0.1 mM to about 15 mM, about 0.2 mM to about 15 mM, about 0.3 mM to about 15 mM, about 0.4 mM to about 15 mM, about 0.5 mM to about 15 mM, about 0.6 mM to about 15 mM, about 0.7 mM to about 15 mM, about 0.8 mM to about 15 mM, about 0.9 mM to about 15 mM, about 1.0 mM to about 15 mM, about 0.1 mM to about 10 mM, about 0.2 mM to about 10 mM, about 0.3 mM to about 10 mM, about 0.4 mM to about 10 mM, about 0.5 mM to about 10 mM, about 0.6 mM to about 10 mM, about 0.7 mM to about 10 mM, about 0.8 mM to about 10 mM, about 0.9 mM to about 10 mM, about 1.0 mM to about 10 mM, about 0.1 mM to about 9 mM, about 0.2 mM to about 9 mM, about 0.3 mM to about 9 mM, about 0.4 mM to about 9 mM, about 0.5 mM to about 9 mM, about 0.6 mM to about 9 mM, about 0.7 mM to about 9 mM, about 0.8 mM to about 9 mM, about 0.9 mM to about 9 mM, about 1.0 mM to about 9 mM, about 0.1 mM to about 8 mM, about 0.2 mM to about 8 mM, about 0.3 mM to about 8 mM, about 0.4 mM to about 8 mM, about 0.5 mM to about 8 mM, about 0.6 mM to about 8 mM, about 0.7 mM to about 8 mM, about 0.8 mM to about 8 mM, about 0.9 mM to about 8 mM, about 1.0 mM to about 8 mM, about 0.1 mM to about 7 mM, about 0.2 mM to about 7 mM, about 0.3 mM to about 7 mM, about 0.4 mM to about 7 mM, about 0.5 mM to about 7 mM, about 0.6 mM to about 7 mM, about 0.7 mM to about 7 mM, about 0.8 mM to about 7 mM, about 0.9 mM to about 7 mM, about 1.0 mM to about 7 mM, about 0.1 mM to about 6 mM, about 0.2 mM to about 6 mM, about 0.3 mM to about 6 mM, about 0.4 mM to about 6 mM, about 0.5 mM to about 6 mM, about 0.6 mM to about 6 mM, about 0.7 mM to about 6 mM, about 0.8 mM to about 6 mM, about 0.9 mM to about 6 mM, about 1.0 mM to about 6 mM, about 0.1 mM to about 5 mM, about 0.2 mM to about 5 mM, about 0.3 mM to about 5 mM, about 0.4 mM to about 5 mM, about 0.5 mM to about 5 mM, about 0.6 mM to about 5 mM, about 0.7 mM to about 5 mM, about 0.8 mM to about 5 mM, about 0.9 mM to about 5 mM, about 1.0 mM to about 5 mM, about 0.1 mM to about 4 mM, about 0.2 mM to about 4 mM, about 0.3 mM to about 4 mM, about 0.4 mM to about 4 mM, about 0.5 mM to about 4 mM, about 0.6 mM to about 4 mM, about 0.7 mM to about 4 mM, about 0.8 mM to about 4 mM, about 0.9 mM to about 4 mM, about 1.0 mM to about 4 mM, about 0.1 mM to about 3 mM, about 0.2 mM to about 3 mM, about 0.3 mM to about 3 mM, about 0.4 mM to about 3 mM, about 0.5 mM to about 3 mM, about 0.6 mM to about 3 mM, about 0.7 mM to about 3 mM, about 0.8 mM to about 3 mM, about 0.9 mM to about 3 mM, about 1.0 mM to about 3 mM; or a concentration or set of concentrations within the foregoing concentration ranges; or a sub-range of any of the foregoing concentration ranges.

In a further aspect, the concentration of the third modulator component can be from about 0.1 mM to about 30 mM, about 0.2 mM to about 30 mM, about 0.3 mM to about 30 mM, about 0.4 mM to about 30 mM, about 0.5 mM to about 30 mM, about 0.6 mM to about 30 mM, about 0.7 mM to about 30 mM, about 0.8 mM to about 30 mM, about 0.9 mM to about 30 mM, about 1.0 mM to about 30 mM, about 0.1 mM to about 25 mM, about 0.2 mM to about 25 mM, about 0.3 mM to about 25 mM, about 0.4 mM to about 25 mM, about 0.5 mM to about 25 mM, about 0.6 mM to about 25 mM, about 0.7 mM to about 25 mM, about 0.8 mM to about 25 mM, about 0.9 mM to about 25 mM, about 1.0 mM to about 25 mM, about 0.1 mM to about 20 mM, about 0.2 mM to about 20 mM, about 0.3 mM to about 20 mM, about 0.4 mM to about 20 mM, about 0.5 mM to about 20 mM, about 0.6 mM to about 20 mM, about 0.7 mM to about 20 mM, about 0.8 mM to about 20 mM, about 0.9 mM to about 20 mM, about 1.0 mM to about 20 mM, about 0.1 mM to about 15 mM, about 0.2 mM to about 15 mM, about 0.3 mM to about 15 mM, about 0.4 mM to about 15 mM, about 0.5 mM to about 15 mM, about 0.6 mM to about 15 mM, about 0.7 mM to about 15 mM, about 0.8 mM to about 15 mM, about 0.9 mM to about 15 mM, about 1.0 mM to about 15 mM, about 0.1 mM to about 10 mM, about 0.2 mM to about 10 mM, about 0.3 mM to about 10 mM, about 0.4 mM to about 10 mM, about 0.5 mM to about 10 mM, about 0.6 mM to about 10 mM, about 0.7 mM to about 10 mM, about 0.8 mM to about 10 mM, about 0.9 mM to about 10 mM, about 1.0 mM to about 10 mM, about 0.1 mM to about 9 mM, about 0.2 mM to about 9 mM, about 0.3 mM to about 9 mM, about 0.4 mM to about 9 mM, about 0.5 mM to about 9 mM, about 0.6 mM to about 9 mM, about 0.7 mM to about 9 mM, about 0.8 mM to about 9 mM, about 0.9 mM to about 9 mM, about 1.0 mM to about 9 mM, about 0.1 mM to about 8 mM, about 0.2 mM to about 8 mM, about 0.3 mM to about 8 mM, about 0.4 mM to about 8 mM, about 0.5 mM to about 8 mM, about 0.6 mM to about 8 mM, about 0.7 mM to about 8 mM, about 0.8 mM to about 8 mM, about 0.9 mM to about 8 mM, about 1.0 mM to about 8 mM, about 0.1 mM to about 7 mM, about 0.2 mM to about 7 mM, about 0.3 mM to about 7 mM, about 0.4 mM to about 7 mM, about 0.5 mM to about 7 mM, about 0.6 mM to about 7 mM, about 0.7 mM to about 7 mM, about 0.8 mM to about 7 mM, about 0.9 mM to about 7 mM, about 1.0 mM to about 7 mM, about 0.1 mM to about 6 mM, about 0.2 mM to about 6 mM, about 0.3 mM to about 6 mM, about 0.4 mM to about 6 mM, about 0.5 mM to about 6 mM, about 0.6 mM to about 6 mM, about 0.7 mM to about 6 mM, about 0.8 mM to about 6 mM, about 0.9 mM to about 6 mM, about 1.0 mM to about 6 mM, about 0.1 mM to about 5 mM, about 0.2 mM to about 5 mM, about 0.3 mM to about 5 mM, about 0.4 mM to about 5 mM, about 0.5 mM to about 5 mM, about 0.6 mM to about 5 mM, about 0.7 mM to about 5 mM, about 0.8 mM to about 5 mM, about 0.9 mM to about 5 mM, about 1.0 mM to about 5 mM, about 0.1 mM to about 4 mM, about 0.2 mM to about 4 mM, about 0.3 mM to about 4 mM, about 0.4 mM to about 4 mM, about 0.5 mM to about 4 mM, about 0.6 mM to about 4 mM, about 0.7 mM to about 4 mM, about 0.8 mM to about 4 mM, about 0.9 mM to about 4 mM, about 1.0 mM to about 4 mM, about 0.1 mM to about 3 mM, about 0.2 mM to about 3 mM, about 0.3 mM to about 3 mM, about 0.4 mM to about 3 mM, about 0.5 mM to about 3 mM, about 0.6 mM to about 3 mM, about 0.7 mM to about 3 mM, about 0.8 mM to about 3 mM, about 0.9 mM to about 3 mM, about 1.0 mM to about 3 mM; or a concentration or set of concentrations within the foregoing concentration ranges; or a sub-range of any of the foregoing concentration ranges.

The disclosed beverage or food product can be at a suitable pH, e.g. a pH of from about pH 2 to about pH 9. In some instances, it may be desirable, e.g., to optimize a Taste Quality Metric such as sweetness linger, and/or body/mouthfeel, to use a lower pH, such as a pH of from about pH 2.0 to about pH 7.0, about pH 2.0 to about pH 6.9, about pH 2.0 to about pH 6.8, about pH 2.0 to about pH 6.7, about pH 2.0 to about pH 6.6, about pH 2.0 to about pH 6.5, about pH 2.0 to about pH 6.4, about pH 2.0 to about pH 6.3, about pH 2.0 to about pH 6.2, about pH 2.0 to about pH 6.1, about pH 2.0 to about pH 6.0, about pH 2.0 to about pH 5.9, about pH 2.0 to about pH 5.8, about pH 2.0 to about pH 5.7, about pH 2.0 to about pH 5.6, about pH 2.0 to about pH 5.5, about pH 2.0 to about pH 5.4, about pH 2.0 to about pH 5.3, about pH 2.0 to about pH 5.2, about pH 2.0 to about pH 5.1, about pH 2.0 to about pH 5.0, about pH 2.0 to about pH 4.9, about pH 2.0 to about pH 4.8, about pH 2.0 to about pH 4.7, about pH 2.0 to about pH 4.6, about pH 2.0 to about pH 4.5, about pH 2.0 to about pH 4.4, about pH 2.0 to about pH 4.3, about pH 2.0 to about pH 4.2, about pH 2.0 to about pH 4.1, about pH 2.0 to about pH 4.0, about pH 2.0 to about pH 3.9, about pH 2.0 to about pH 3.8, about pH 2.0 to about pH 3.7, about pH 2.0 to about pH 3.6, about pH 2.0 to about pH 3.5, about pH 2.0 to about pH 3.4, about pH 2.0 to about pH 3.3, about pH 2.0 to about pH 3.2, about pH 2.0 to about pH 3.1, about pH 2.0 to about pH 3.0, about pH 2.1 to about pH 7.0, about pH 2.1 to about pH 6.9, about pH 2.1 to about pH 6.8, about pH 2.1 to about pH 6.7, about pH 2.1 to about pH 6.6, about pH 2.1 to about pH 6.5, about pH 2.1 to about pH 6.4, about pH 2.1 to about pH 6.3, about pH 2.1 to about pH 6.2, about pH 2.1 to about pH 6.1, about pH 2.1 to about pH 6.0, about pH 2.1 to about pH 5.9, about pH 2.1 to about pH 5.8, about pH 2.1 to about pH 5.7, about pH 2.1 to about pH 5.6, about pH 2.1 to about pH 5.5, about pH 2.1 to about pH 5.4, about pH 2.1 to about pH 5.3, about pH 2.1 to about pH 5.2, about pH 2.1 to about pH 5.1, about pH 2.1 to about pH 5.0, about pH 2.1 to about pH 4.9, about pH 2.1 to about pH 4.8, about pH 2.1 to about pH 4.7, about pH 2.1 to about pH 4.6, about pH 2.1 to about pH 4.5, about pH 2.1 to about pH 4.4, about pH 2.1 to about pH 4.3, about pH 2.1 to about pH 4.2, about pH 2.1 to about pH 4.1, about pH 2.1 to about pH 4.0, about pH 2.1 to about pH 3.9, about pH 2.1 to about pH 3.8, about pH 2.1 to about pH 3.7, about pH 2.1 to about pH 3.6, about pH 2.1 to about pH 3.5, about pH 2.1 to about pH 3.4, about pH 2.1 to about pH 3.3, about pH 2.1 to about pH 3.2, about pH 2.1 to about pH 3.1, about pH 2.1 to about pH 3.0, about pH 2.2 to about pH 7.0, about pH 2.2 to about pH 6.9, about pH 2.2 to about pH 6.8, about pH 2.2 to about pH 6.7, about pH 2.2 to about pH 6.6, about pH 2.2 to about pH 6.5, about pH 2.2 to about pH 6.4, about pH 2.2 to about pH 6.3, about pH 2.2 to about pH 6.2, about pH 2.2 to about pH 6.1, about pH 2.2 to about pH 6.0, about pH 2.2 to about pH 5.9, about pH 2.2 to about pH 5.8, about pH 2.2 to about pH 5.7, about pH 2.2 to about pH 5.6, about pH 2.2 to about pH 5.5, about pH 2.2 to about pH 5.4, about pH 2.2 to about pH 5.3, about pH 2.2 to about pH 5.2, about pH 2.2 to about pH 5.1, about pH 2.2 to about pH 5.0, about pH 2.2 to about pH 4.9, about pH 2.2 to about pH 4.8, about pH 2.2 to about pH 4.7, about pH 2.2 to about pH 4.6, about pH 2.2 to about pH 4.5, about pH 2.2 to about pH 4.4, about pH 2.2 to about pH 4.3, about pH 2.2 to about pH 4.2, about pH 2.2 to about pH 4.1, about pH 2.2 to about pH 4.0, about pH 2.2 to about pH 3.9, about pH 2.2 to about pH 3.8, about pH 2.2 to about pH 3.7, about pH 2.2 to about pH 3.6, about pH 2.2 to about pH 3.5, about pH 2.2 to about pH 3.4, about pH 2.2 to about pH 3.3, about pH 2.2 to about pH 3.2, about pH 2.2 to about pH 3.1, about pH 2.2 to about pH 3.0, about pH 2.3 to about pH 7.0, about pH 2.3 to about pH 6.9, about pH 2.3 to about pH 6.8, about pH 2.3 to about pH 6.7, about pH 2.3 to about pH 6.6, about pH 2.3 to about pH 6.5, about pH 2.3 to about pH 6.4, about pH 2.3 to about pH 6.3, about pH 2.3 to about pH 6.2, about pH 2.3 to about pH 6.1, about pH 2.3 to about pH 6.0, about pH 2.3 to about pH 5.9, about pH 2.3 to about pH 5.8, about pH 2.3 to about pH 5.7, about pH 2.3 to about pH 5.6, about pH 2.3 to about pH 5.5, about pH 2.3 to about pH 5.4, about pH 2.3 to about pH 5.3, about pH 2.3 to about pH 5.2, about pH 2.3 to about pH 5.1, about pH 2.3 to about pH 5.0, about pH 2.3 to about pH 4.9, about pH 2.3 to about pH 4.8, about pH 2.3 to about pH 4.7, about pH 2.3 to about pH 4.6, about pH 2.3 to about pH 4.5, about pH 2.3 to about pH 4.4, about pH 2.3 to about pH 4.3, about pH 2.3 to about pH 4.2, about pH 2.3 to about pH 4.1, about pH 2.3 to about pH 4.0, about pH 2.3 to about pH 3.9, about pH 2.3 to about pH 3.8, about pH 2.3 to about pH 3.7, about pH 2.3 to about pH 3.6, about pH 2.3 to about pH 3.5, about pH 2.3 to about pH 3.4, about pH 2.3 to about pH 3.3, about pH 2.3 to about pH 3.2, about pH 2.3 to about pH 3.1, about pH 2.3 to about pH 3.0, about pH 2.4 to about pH 7.0, about pH 2.4 to about pH 6.9, about pH 2.4 to about pH 6.8, about pH 2.4 to about pH 6.7, about pH 2.4 to about pH 6.6, about pH 2.4 to about pH 6.5, about pH 2.4 to about pH 6.4, about pH 2.4 to about pH 6.3, about pH 2.4 to about pH 6.2, about pH 2.4 to about pH 6.1, about pH 2.4 to about pH 6.0, about pH 2.4 to about pH 5.9, about pH 2.4 to about pH 5.8, about pH 2.4 to about pH 5.7, about pH 2.4 to about pH 5.6, about pH 2.4 to about pH 5.5, about pH 2.4 to about pH 5.4, about pH 2.4 to about pH 5.3, about pH 2.4 to about pH 5.2, about pH 2.4 to about pH 5.1, about pH 2.4 to about pH 5.0, about pH 2.4 to about pH 4.9, about pH 2.4 to about pH 4.8, about pH 2.4 to about pH 4.7, about pH 2.4 to about pH 4.6, about pH 2.4 to about pH 4.5, about pH 2.4 to about pH 4.4, about pH 2.4 to about pH 4.3, about pH 2.4 to about pH 4.2, about pH 2.4 to about pH 4.1, about pH 2.4 to about pH 4.0, about pH 2.4 to about pH 3.9, about pH 2.4 to about pH 3.8, about pH 2.4 to about pH 3.7, about pH 2.4 to about pH 3.6, about pH 2.4 to about pH 3.5, about pH 2.4 to about pH 3.4, about pH 2.4 to about pH 3.3, about pH 2.4 to about pH 3.2, about pH 2.4 to about pH 3.1, about pH 2.4 to about pH 3.0, about pH 2.5 to about pH 7.0, about pH 2.5 to about pH 6.9, about pH 2.5 to about pH 6.8, about pH 2.5 to about pH 6.7, about pH 2.5 to about pH 6.6, about pH 2.5 to about pH 6.5, about pH 2.5 to about pH 6.4, about pH 2.5 to about pH 6.3, about pH 2.5 to about pH 6.2, about pH 2.5 to about pH 6.1, about pH 2.5 to about pH 6.0, about pH 2.5 to about pH 5.9, about pH 2.5 to about pH 5.8, about pH 2.5 to about pH 5.7, about pH 2.5 to about pH 5.6, about pH 2.5 to about pH 5.5, about pH 2.5 to about pH 5.4, about pH 2.5 to about pH 5.3, about pH 2.5 to about pH 5.2, about pH 2.5 to about pH 5.1, about pH 2.5 to about pH 5.0, about pH 2.5 to about pH 4.9, about pH 2.5 to about pH 4.8, about pH 2.5 to about pH 4.7, about pH 2.5 to about pH 4.6, about pH 2.5 to about pH 4.5, about pH 2.5 to about pH 4.4, about pH 2.5 to about pH 4.3, about pH 2.5 to about pH 4.2, about pH 2.5 to about pH 4.1, about pH 2.5 to about pH 4.0, about pH 2.5 to about pH 3.9, about pH 2.5 to about pH 3.8, about pH 2.5 to about pH 3.7, about pH 2.5 to about pH 3.6, about pH 2.5 to about pH 3.5, about pH 2.5 to about pH 3.4, about pH 2.5 to about pH 3.3, about pH 2.5 to about pH 3.2, about pH 2.5 to about pH 3.1, about pH 2.5 to about pH 3.0; or any pH value or sub-range within the foregoing ranges.

Flavoring Compositions, Flavor Modifier Compositions and Flavorings with Modifying Properties

The disclosed taste modulator compositions can be used as flavor or taste modifiers, such as a flavoring or flavoring agents and flavor or taste enhancers, more particularly, bitterness modifiers and bitterness inhibitors for foods, beverages, and other comestible or orally administered medicinal or nutraceutical products or compositions. In some aspects, the disclosed taste modulator compositions can be used without a flavor, flavor modifier, flavor agent, flavor enhancer, or flavoring with modifying properties for comestible products. In other aspects, the disclosed taste modulator compositions are used with one or more a flavor, flavor modifier, flavor agent, flavor enhancer, or flavoring with modifying properties for comestible products. In a further aspect, the disclosed taste modulator compositions can be used in a flavoring with modifying properties composition comprising a disclosed modulator composition and one or more flavor, flavor modifier, flavor agent, flavor enhancer, or flavoring.

The suitable amount of the disclosed taste modulator compositions to use with one or more flavor, flavor modifier, flavor agent, flavor enhancer, or flavoring with modifying properties can be assessed and further optimized using a suitable sensory testing methodology, e.g., the sensory testing methodology described herein below for sensory assessment of bitterness. Moreover, such a sensory test methodology can be useful in identifying suitable disclosed taste modulator compositions (and concentrations of same) to be used in combination with flavor, flavor modifier, flavor agent, flavor enhancer, or flavoring with modifying properties. The sensory test methodology described herein below can be well quantified and controlled by tasting the candidate compounds in aqueous solutions, as compared to control aqueous solution, or alternatively by tasting the disclosed taste modulator compositions of the disclosure in actual food compositions.

In various aspects, the disclosed taste modulator compositions, either alone or in combination with one or more flavor, flavor modifier, flavor agent, flavor enhancer, or flavoring with modifying properties have application in foods, beverages and other comestible compositions wherein in which it is desirable to modify the bitterness profile thereof. These compositions include compositions for human and animal consumption. This includes food or drinks (liquids) for consumption by agricultural animals, pets and zoo animals. Those of ordinary skill in the art of preparing and selling comestible compositions (e.g. edible foods or beverages, or precursors or flavor modifiers thereof) are well aware of a large variety of classes, subclasses and species of the comestible compositions, and utilize well-known and recognized terms of art to refer to those comestible compositions while endeavoring to prepare and sell various of those comestible compositions. Such a list of terms of art is enumerated below, and it is specifically contemplated hereby that the disclosed taste modulator compositions could be used to modify or inhibit the bitterness profile or flavor of the following list comestible compositions, either singly or in all reasonable combinations or mixtures thereof: one or more confectioneries, chocolate confectionery, tablets, boxed assortments, standard boxed assortments, twist wrapped miniatures, seasonal chocolate, chocolate with toys, other chocolate confectionery, mints, standard mints, power mints, boiled sweets, pastilles, gums, jellies and chews, toffees, caramels and nougat, medicated confectionery, lollipops, licorice, other sugar confectionery, gum, chewing gum, gum with one or more sweeteners, sugar-free gum, functional gum, bubble gum, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, cereals, family breakfast cereals, flakes, muesli, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yogurt, artisanal ice cream, dairy products, milk, fresh/pasteurized milk, full fat fresh/pasteurized milk, semi skimmed fresh/pasteurized milk, full fat milk, semi skimmed milk, fat-free milk, goat milk, condensed/evaporated milk, plain condensed/evaporated milk, flavored, functional and other condensed milk, flavored milk drinks, dairy only flavored milk drinks, flavored milk drinks with fruit juice, soy milk, sour milk drinks, fermented dairy drinks, coffee whiteners, powder milk, flavored powder milk drinks, cream, cheese, processed cheese, spreadable processed cheese, non-spreadable processed cheese, unprocessed cheese, spreadable unprocessed cheese, hard cheese, packaged hard cheese, unpackaged hard cheese, yogurt, plain/natural yogurt, flavored yogurt, fruited yogurt, probiotic yogurt, drinking yogurt, regular drinking yogurt, probiotic drinking yogurt, chilled and shelf-stable desserts, dairy-based desserts, soy-based desserts, chilled snacks, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and Seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, and yeast-based spreads.

In a further aspect, the disclosed taste modulator compositions can be used to modify or inhibit the bitterness flavor or profile of one or more of the following sub-groups of comestible compositions: confectioneries, bakery products, ice creams, dairy products, sweet snacks, snack bars, meal replacement products, ready meals, soups, pastas, noodles, canned foods, frozen foods, dried foods, chilled foods, oils and fats, baby foods, or spreads, or a mixture thereof.

In general an ingestible composition will be produced that contains a sufficient amount of the disclosed taste modulator compositions described hereinabove to produce a composition having the desired modulation or reduction in flavor or taste characteristics such as “bitterness” taste characteristics.

Typically one or more of the disclosed taste modulator compositions can be added to the comestible product, so that the bitterness flavor modified comestible product has an decreased bitterness taste as compared to the comestible product prepared without the disclosed taste modulator compositions, as judged by human beings or animals in general, or in the case of formulations testing, as judged by a majority of a panel of human taste testers, via procedures described elsewhere herein.

The concentration of taste modulator component useful to modulate or inhibit the bitterness flavor of the comestible product or composition will of course vary dependent on many variables, including the specific type of ingestible composition, what other compounds are already present and the concentrations thereof, the amount of presence of other flavors with modifying properties, and the enhancer effect of the particular compound on such bitterness taste properties. As noted, an application of the disclosed taste modulator compositions is for modulating (reducing, mitigating or inhibiting) the bitter tastes or other bitterness properties of other natural or synthetic tastants. A broad range of concentrations of the disclosed taste modulator compositions can be employed to provide such modulation of bitter taste as described herein elsewhere.

Examples of foods and beverages wherein the disclosed taste modulator compositions may be incorporated included by way of example the Wet Soup Category, the Dehydrated and Culinary Food Category, the Beverage Category, the Frozen Food Category, the Snack Food Category, and seasonings or seasoning blends. “Wet Soup Category” means wet/liquid soups regardless of concentration or container, including frozen Soups. For the purpose of this definition soup(s) means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consomme) to sauces (cream or cheese-based soups).

“Dehydrated and Culinary Food Category” means: (i) Cooking aid products such as: powders, granules, pastes, concentrated liquid products, including concentrated bouillon, bouillon and bouillon like products in pressed cubes, tablets or powder or granulated form, which are sold separately as a finished product or as an ingredient within a product, sauces and recipe mixes (regardless of technology); (ii) Meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) Meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid or frozen.

“Beverage Category” means beverages, beverage mixes and concentrates, including but not limited to, alcoholic and non-alcoholic ready to drink and dry powdered beverages. Other examples of foods and beverages wherein compounds according to the disclosure may be incorporated included byway of example carbonated and non-carbonated beverages, e.g., sodas, fruit or vegetable juices, alcoholic and non-alcoholic beverages, confectionary products, e.g., cakes, cookies, pies, candies, chewing gums, gelatins, ice creams, sorbets, puddings, jams, jellies, salad dressings, and other condiments, cereal, and other breakfast foods, canned fruits and fruit sauces and the like.

Additionally, the subject compounds can be used in flavor preparations to be added to foods and beverages. In preferred instances the composition will comprise another flavor or taste modifier such as a sweet or umami tastant.

Accordingly, in some aspects, the disclosure relates to methods for modulating the bitter taste of a comestible product comprising: a) providing at least one comestible product, or a precursor thereof, and b) combining the comestible product or precursor thereof with at least a bitter modulating amount of the disclosed taste modulator compositions, so as to form a modified comestible product.

The disclosure also relates to the modified comestible products produced by such processes, and similar processes for producing comestible products well known to those of ordinary skill in the art, especially if such compositions comprise other flavorings with modifying properties. The disclosed taste modulator compositions can be combined with or applied to the comestible or medicinal products or precursor thereof in any of innumerable ways known to cooks the world over, or producers of comestible or medicinal products. For example, the disclosed taste modulator compositions can be dissolved in or dispersed in or one of many known comestibly acceptable liquids, solids, or other carriers, such as water at neutral, acidic, or basic pH, fruit or vegetable juices, vinegar, marinades, beer, wine, natural water/fat emulsions such as milk or condensed milk, edible oils and shortenings, fatty acids, certain low molecular weight oligomers of propylene glycol, glyceryl esters of fatty acids, and dispersions or emulsions of such hydrophobic substances in aqueous media, salts such as sodium chloride, vegetable flours, solvents such as ethanol, solid edible diluents such as vegetable powders or flours, and the like, and then combined with precursors of the comestible or medicinal products, or applied directly to the comestible or medicinal products.

Steviol glycosides, Stevia-derived sweeteners, and Stevia-derived extracts provide sweetness and other taste attributes at a higher than certain threshold level of concentrations in water. Below the threshold level of concentration, the steviol glycosides, Stevia-derived sweeteners, Stevia-derived extracts, and their mixtures have no recognizable sweetness taste, but such Stevia extract below the threshold level of significant sweetness recognition can be associated with sweet and flavor profile modification in food and beverage applications.

Accordingly, in various aspects, the present disclosure pertains to flavorings with modifying properties comprising a disclosed taste modulator composition and at least one steviol glycoside, Stevia-derived sweetener, Stevia-derived extract, and combinations thereof, such that the at least one steviol glycoside, Stevia-derived sweetener, Stevia-derived extract, and combinations thereof is present in an amount less than 1.5 wt % sucrose equivalent. In some aspects, the least one steviol glycoside, Stevia-derived sweetener, Stevia-derived extract, and combinations thereof comprises one or more glucosylated steviol glycoside.

In other aspects, the present disclosure pertains to flavorings with modifying properties comprising a disclosed taste modulator composition and at least one Siraitia grosvenorii (luo han guo or monk fruit)-derived sweetener, extract, juice, and combinations thereof, such that the at least least one Siraitia grosvenorii (luo han guo or monk fruit)-derived sweetener, extract, juice, and combinations thereof is present in an amount less than 1.5 wt % sucrose equivalent.

In further aspects, the present disclosure pertains to flavorings with modifying properties comprising: (a) a disclosed taste modulator composition; (b) at least one Siraitia grosvenorii (luo han guo or monk fruit)-derived sweetener, extract, juice, and combinations thereof, such that the at least one Siraitia grosvenorii (luo han guo or monk fruit)-derived sweetener, extract, juice, and combinations thereof is present in an amount less than 1.5 wt % sucrose equivalent; and (c) at least one steviol glycoside, Stevia-derived sweetener, Stevia-derived extract, and combinations thereof, such that the at least one steviol glycoside, Stevia-derived sweetener, Stevia-derived extract, and combinations thereof is present in an amount less than 1.5 wt % sucrose equivalent.

In some aspects, the least one steviol glycoside, Stevia-derived sweetener, Stevia-derived extract, and combinations thereof comprises one or more glucosylated steviol glycoside.

The present disclosure is directed to a taste and flavor modifying composition. The disclosed taste and flavor modifying composition, which can modify the intensity of a taste and/or a flavor in a food or beverage product, comprises a disclosed taste modulator composition and at least one steviol glycoside, Stevia-derived sweetener, Stevia-derived extract, and combinations thereof, comprising one or more steviol glycosides with other water soluble molecules derived from Stevia plant, such as non-limiting examples of plant glycosides, flavonoids, labdane diterpene, triterpenes.

The present disclosure is also directed to a food or beverage product having an intense taste and flavor profile, wherein the food or beverage product comprises a disclosed taste and flavor modifying composition, which can modify the intensity of a taste and/or a flavor in a food or beverage product, comprises a disclosed taste modulator composition and at least one steviol glycoside, Stevia-derived sweetener, Stevia-derived extract, and combinations thereof, comprising one or more steviol glycosides with other water soluble molecules derived from Stevia plant, such as non-limiting examples of plant glycosides, flavonoids, labdane diterpene, triterpenes. A wide range of food and beverage products, such as, but not limited to, carbonated soft drinks, fruit juices, dairy foods, dairy beverages, baked goods, cereal products, snack foods, and table top sweeteners, may be made in accordance with the present disclosure. The taste and flavor profile of a food or beverage product comprising a taste and flavor modifying composition, wherein the taste and flavor modifying composition comprising the Stevia extract of steviol glycosides and water soluble molecules derived from Stevia plant, may be more intense than a comparative taste and flavor profile of a comparative food or beverage product which does not include the taste and flavor modifying composition. Moreover, the mouthfeel and overall taste perception of a food or beverage product comprising the taste and flavor modifying composition, wherein the taste and flavor enhancing composition includes the complex mixture of steviol glycosides and water soluble molecules, may be improved in relation to a mouthfeel and overall taste perception of a comparative food or beverage product which does not include the taste and flavor enhancing composition.

The present disclosure is further directed to a method of increasing the taste and flavor intensity of a food or beverage product, comprising the step of adding a taste and flavor enhancing composition to the food or beverage product, wherein the taste and flavor modifying composition comprising the Stevia extract of steviol glycosides and water soluble molecules derived from Stevia plant. The present disclosure is also directed to a method of improving the organoleptic properties of a food or beverage product comprising a high fructose syrup, comprising the step of adding the taste and flavor modifying composition to the food or beverage product. For example, adding the taste and flavor modifying composition may cause the high fructose syrup, such as high fructose corn syrup, to taste more like sugar. Also, if the high fructose syrup is high fructose corn syrup 42 (HFCS 42), adding the taste and flavor enhancing composition may cause the HFCS 42 to taste more like high fructose corn syrup 55 (HFCS 55).

The present disclosure is further directed to a method of increasing the taste and flavor intensity of a medical food and pharma product, comprising the step of adding a taste and flavor modifying composition to the food or beverage product, wherein the taste and flavor modifying composition comprising the Stevia extract of selected steviol glycosides and water soluble molecules derived from Stevia plant. The present disclosure is also directed to a method of improving the organoleptic properties of a medical food or pharma product containing functional food ingredients like vitamins, minerals and amino acids, comprising the step of adding the taste and flavor modifying composition to the food or beverage product. For example, adding the taste and flavor modifying composition may cause the off-taste due to vitamins, mineral, amino acids and other non-limiting functional ingredients, to improve taste and palatability.

The present disclosure is also directed to a method of making a taste and flavor enhancing composition, comprising: extracting steviol glycosides and other water soluble molecules from leaves of a Stevia rebaudiana plant, and separating the excess steviol glycosides than the amount and type of steviol glycosides required to contribute the taste and flavor modifying characteristics of the Stevia extract, and adding to the extract prepared a disclosed taste modulator composition. The present disclosure also pertains to kits for making a taste and flavor enhancing composition, comprising: (a) a disclosed taste modulator composition; and (b) instructions to utilize the disclosed taste modulator composition with one or more disclosed sweet flavorings, such as at least one Siraitia grosvenorii (luo han guo or monk fruit)-derived sweetener, extract, juice, and combinations thereof, such that the at least one Siraitia grosvenorii (luo han guo or monk fruit)-derived sweetener, extract, juice, and combinations thereof is present in an amount less than 1.5 wt % sucrose equivalent and/or at least one Siraitia grosvenorii (luo han guc or monk fruit)-derived sweetener, extract, juice, and combinations thereof, such that the at least one Siraitia grosvenorii (luo han guo or monk fruit)-derived sweetener, extract, juice, and combinations thereof is present in an amount less than 1.5 wt % sucrose equivalent. The kit can further comprise one or more disclosed sweet flavorings, such as at least one Siraitia grosvenorii (luo han guo or monk fruit)-derived sweetener, extract, juice, and combinations thereof, such that the at least one Siraitia grosvenorii (luo han guo or monk fruit)-derived sweetener, extract, juice, and combinations thereof and/or at least one Siraitia grosvenorii (luo han guo or monk fruit)-derived sweetener, extract, juice, and combinations thereof.

The present disclosure is also directed to a taste and flavor profile enhancing composition. The composition includes a disclosed glucosylated steviol glycoside composition and a disclosed taste modulator composition which can enhance the intensity of a taste and/or a flavor in a food or beverage product. In some aspects, the glucosylated steviol glycosides may include a plurality of glucose units. For example, the glucosylated steviol glycosides may include three, four, five, or more than five glucose units. As used herein, “a disclosed glucosylated steviol glycoside composition” refers to any glucosylated steviol composition comprise one or more steviol or steviol derivative as described herein throughout.

The present disclosure is also directed to a food or beverage product having an intense taste and flavor profile, wherein the food or beverage product includes a taste and flavor enhancing composition comprising a glucosylated steviol glycoside composition and a disclosed taste modulator composition. A wide range of food and beverage products, such as, but not limited to, carbonated soft drinks, fruit juices, dairy foods, dairy beverages, baked goods, cereal products, and table top sweeteners, may be made in accordance with the present disclosure. The taste and flavor profile of a food or beverage product including a taste and flavor enhancing composition, wherein the taste and flavor enhancing composition includes a disclosed glucosylated steviol glycoside composition and a disclosed taste modulator composition, may be more intense than a comparative taste and flavor profile of a comparative food or beverage product which does not include the taste and flavor enhancing composition. Moreover, the mouthfeel of a food or beverage product including the taste and flavor enhancing composition, wherein the taste and flavor enhancing composition includes a disclosed glucosylated steviol glycoside composition and a disclosed taste modulator composition, may be improved in relation to a mouthfeel of a comparative food or beverage product which does not include the taste and flavor enhancing composition.

The present disclosure is further directed to a method of increasing the taste and flavor intensity of a food or beverage product, including the step of adding a taste and flavor enhancing composition to the food or beverage product, wherein the taste and flavor enhancing composition includes a disclosed glucosylated steviol glycoside composition and a disclosed taste modulator composition. The present disclosure is also directed to a method of improving the organoleptic properties of a food or beverage product including a high fructose syrup, including the step of adding the taste and flavor enhancing composition to the food or beverage product. For example, adding the taste and flavor enhancing composition may cause the high fructose syrup, such as high fructose corn syrup, to taste more like sugar. Also, if the high fructose syrup is high fructose corn syrup 42 (HFCS 42), adding the taste and flavor enhancing composition may cause the HFCS 42 to taste more like high fructose corn syrup 55 (HFCS 55).

The present disclosure is further directed to a method of making a food or beverage product, including: adding a taste and flavor enhancing composition including a disclosed glucosylated steviol glycoside composition and a disclosed taste modulator composition, and adding a reduced amount of erythritol, wherein the reduced amount of erythritol is less than the amount of erythritol in a comparative food or beverage composition which does not include the taste and flavor enhancing composition. The mouthfeel of the food or beverage product is similar to the mouthfeel of the comparative food or beverage product, even though the comparative food or beverage product contains a higher level of erythritol.

The present disclosure is also directed to a taste and flavor profile enhancing composition. The composition includes a disclosed glucosylated steviol glycoside composition, a disclosed monk fruit composition, and a disclosed taste modulator composition which can enhance the intensity of a taste and/or a flavor in a food or beverage product. In some aspects, the glucosylated steviol glycosides may include a plurality of glucose units. For example, the glucosylated steviol glycosides may include three, four, five, or more than five glucose units. As used herein, “a disclosed glucosylated steviol glycoside composition” refers to any glucosylated steviol composition comprise one or more steviol or steviol derivative as described herein throughout. As used herein, “a disclosed monk fruit composition” refers to any monk fruit juice, extract, composition, or mixture, including a juice, extract, composition, or mixture comprising mogroside V, as described herein throughout.

The present disclosure is also directed to a food or beverage product having an intense taste and flavor profile, wherein the food or beverage product includes a taste and flavor enhancing composition comprising a glucosylated steviol glycoside composition and a disclosed taste modulator composition. A wide range of food and beverage products, such as, but not limited to, carbonated soft drinks, fruit juices, dairy foods, dairy beverages, baked goods, cereal products, and table top sweeteners, may be made in accordance with the present disclosure. The taste and flavor profile of a food or beverage product including a taste and flavor enhancing composition, wherein the taste and flavor enhancing composition includes a disclosed glucosylated steviol glycoside composition, a disclosed monk fruit composition, and a disclosed taste modulator composition, may be more intense than a comparative taste and flavor profile of a comparative food or beverage product which does not include the taste and flavor enhancing composition. Moreover, the mouthfeel of a food or beverage product including the taste and flavor enhancing composition, wherein the taste and flavor enhancing composition includes a disclosed glucosylated steviol glycoside composition, a disclosed monk fruit composition, and a disclosed taste modulator composition, may be improved in relation to a mouthfeel of a comparative food or beverage product which does not include the taste and flavor enhancing composition.

The present disclosure is further directed to a method of increasing the taste and flavor intensity of a food or beverage product, including the step of adding a taste and flavor composition to the food or beverage product, wherein the taste and flavor enhancing composition includes a disclosed glucosylated steviol glycoside composition, a disclosed monk fruit composition, and a disclosed taste modulator composition. The present disclosure is also directed to a method of improving the organoleptic properties of a food or beverage product including a high fructose syrup, including the step of adding the taste and flavor enhancing composition to the food or beverage product. For example, adding the taste and flavor enhancing composition may cause the high fructose syrup, such as high fructose corn syrup, to taste more like sugar. Also, if the high fructose syrup is high fructose corn syrup 42 (HFCS 42), adding the taste and flavor enhancing composition may cause the HFCS 42 to taste more like high fructose corn syrup 55 (HFCS 55).

The present disclosure is further directed to a method of making a food or beverage product, including: adding a taste and flavor enhancing composition including a disclosed glucosylated steviol glycoside composition, a disclosed monk fruit composition, and a disclosed taste modulator composition, and adding a reduced amount of erythritol, wherein the reduced amount of erythritol is less than the amount of erythritol in a comparative food or beverage composition which does not include the taste and flavor enhancing composition. The mouthfeel of the food or beverage product is similar to the mouthfeel of the comparative food or beverage product, even though the comparative food or beverage product contains a higher level of erythritol.

In a further aspect, the disclosed taste modulator compositions can be used to modify or enhance the flavor, e.g., sweet flavor and/or umami flavor of a food for specified health use, foods with health claims such as foods with nutrient function claims, nutritional supplements and medical foods, while at the same time modulating the bitter taste.

### Methods of Making the Disclosed Bitterness Modified Food and Beverage Compositions

In various aspects, the present disclosure, relates to methods for making a disclosed taste modulator composition comprising: mixing a taste modulator component comprising a first salt having a first cation selected from Na⁺, K⁺, Ca²⁺, and Mg²⁺; and a beverage or food composition having a bitter taste, wherein the mixing is carried out until a mixture of the taste modulator and the beverage or food composition is essentially homogenous. In various aspects, the disclosed methods of making a disclosed taste modulator composition can further comprise mixing a solvent with the taste modulator; and mixing the taste modulator, a beverage or food composition having a bitter taste, and the solvent until a solution or a suspension is essentially homogeneous. In a further aspect, the disclosed methods of making a disclosed taste modulator composition can further comprise spray-drying or lyophilizing the solution or the suspension. In some aspects, the disclosed methods of making a disclosed taste modulator composition can further comprise tableting the mixture of the taste modulator and a concentrated food or beverage composition.

### Products Comprising the Disclosed Taste Modulator Compositions

In various aspects, the present disclosure, relates to products comprising a disclosed taste modulator composition. In various aspects, the product can be a beverage, a food product, a nutraceutical, a concentrated sweetener composition, pharmaceutical, a dietary supplement, a dental hygienic composition, an edible gel composition, a cosmetic product, and a tabletop flavoring. In a further aspect, the present disclosure relates to a tabletop bitterness modifying compositions comprising a disclosed taste modulator composition and one or more disclosed concentrated food or beverage composition.

As used herein, “food product” or “food item” refers to fruits, vegetables, juices, meat products such as ham, bacon and sausage; egg products, fruit concentrates, gelatins and gelatin-like products such as jams, jellies, preserves, and the like; milk products such as ice cream, sour cream, yogurt, and sherbet; icings, syrups including molasses; corn, wheat, rye, soybean, oat, rice and barley products, cereal products, nut meats and nut products, cakes, cookies, confectionaries such as candies, gums, fruit flavored drops, and chocolates, chewing gum, mints, creams, icing, ice cream, pies and breads. “Food product” or “food item” also refers to condiments such as herbs, spices and seasonings, flavor enhancers, such as monosodium glutamate. “Food product” or “food item” further refers to also includes prepared packaged products, such as dietetic sweeteners, liquid sweeteners, tabletop flavorings, granulated flavor mixes which upon reconstitution with water provide non-carbonated drinks, instant pudding mixes, instant coffee and tea, coffee whiteners, malted milk mixes, pet foods, livestock feed, tobacco, and materials for baking applications, such as powdered baking mixes for the preparation of breads, cookies, cakes, pancakes, donuts and the like. “Food product” or “food item” also refers to diet or low-calorie food and beverages containing little or no sucrose.

As used herein, “dietary supplement(s)” refers to compounds intended to supplement the diet and provide nutrients, such as vitamins, minerals, fiber, fatty acids, amino acids, etc. that may be missing or may not be consumed in sufficient quantities in a diet. Any suitable dietary supplement known in the art may be used. Examples of suitable dietary supplements can be, for example, nutrients, vitamins, minerals, fiber, fatty acids, herbs, botanicals, amino acids, and metabolites.

As used herein, “nutraceutical(s)” refers to compounds, which includes any food or part of a food that may provide medicinal or health benefits, including the prevention and/or treatment of disease or disorder (e.g., fatigue, insomnia, effects of aging, memory loss, mood disorders, cardiovascular disease and high levels of cholesterol in the blood, diabetes, osteoporosis, inflammation, autoimmune disorders, etc.). Any suitable nutraceutical known in the art may be used. In some aspects, nutraceuticals can be used as supplements to food and beverages and as pharmaceutical formulations for enteral or parenteral applications which may be solid formulations, such as capsules or tablets, or liquid formulations, such as solutions or suspensions.

In some aspects, dietary supplements and nutraceuticals can further contain protective hydrocolloids (such as gums, proteins, modified starches), binders, film-forming agents, encapsulating agents/materials, wall/shell materials, matrix compounds, coatings, emulsifiers, surface active agents, solubilizing agents (oils, fats, waxes, lecithins, etc.), adsorbents, carriers, fillers, co-compounds, dispersing agents, wetting agents, processing aids (solvents), flowing agents, taste-masking agents, weighting agents, gelling agents, gel-forming agents, antioxidants and antimicrobials.

As used herein, a “gel” refers to a colloidal system in which a network of particles spans the volume of a liquid medium. Although gels mainly are composed of liquids, and thus exhibit densities similar to liquids, gels have the structural coherence of solids due to the network of particles that spans the liquid medium. For this reason, gels generally appear to be solid, jelly-like materials. Gels can be used in a number of applications. For example, gels can be used in foods, paints, and adhesives. Gels that can be eaten are referred to as “edible gel compositions.” Edible gel compositions typically are eaten as snacks, as desserts, as a part of staple foods, or along with staple foods. Examples of suitable edible gel compositions can be, for example, gel desserts, puddings, jams, jellies, pastes, trifles, aspics, marshmallows, gummy candies, and the like. In some aspects, edible gel mixes generally are powdered or granular solids to which a fluid may be added to form an edible gel composition. Examples of suitable fluids can be, for example, water, dairy fluids, dairy analogue fluids, juices, alcohol, alcoholic beverages, and combinations thereof. Examples of suitable dairy fluids can be, for example, milk, cultured milk, cream, fluid whey, and mixtures thereof. Examples of suitable dairy analogue fluids can be, for example, soy milk and non-dairy coffee whitener.

As used herein, the term “gelling ingredient” refers to any material that can form a colloidal system within a liquid medium. Examples of suitable gelling ingredients can be, for example, gelatin, alginate, carageenan, gum, pectin, konjac, agar, food acid, rennet, starch, starch derivatives, and combinations thereof. It is well known to those in the art that the amount of gelling ingredient used in an edible gel mix or an edible gel composition can vary considerably depending on a number of factors such as, for example, the particular gelling ingredient used, the particular fluid base used, and the desired properties of the gel.

Gel mixes and gel compositions of the present disclosure can be prepared by any suitable method known in the art. In some aspects, edible gel mixes and edible gel compositions of the present disclosure can be prepared using other ingredients in addition to a disclosed taste modulator composition and the gelling agent. Examples of other suitable ingredients can be, for example, a food acid, a salt of a food acid, a buffering system, a bulking agent, a sequestrant, a cross-linking agent, one or more flavors, one or more colors, and combinations thereof.

In various aspects, a disclosed beverage can be a carbonated beverage product and a non-carbonated beverage product. The disclosed beverage can also be, for example, a soft drink, a fountain beverage, a frozen beverage; a ready-to-drink beverage; a frozen and ready-to-drink beverage, coffee, tea, a dairy beverage, a powdered soft drink, a liquid concentrate, flavored water, enhanced water, fruit juice, a fruit juice flavored drink, a sport drink, and an energy drink. It is understood that the disclosed beverage products can include one or more beverage ingredients such as, for example, acidulants, fruit juices and/or vegetable juices, pulp, etc., flavorings, coloring, preservatives, vitamins, minerals, electrolytes, erythritol, tagatose, glycerine, and carbon dioxide.

In various aspects, disclosed beverages can have any of numerous different specific formulations or constitutions. The formulation of a beverage product of the present disclosure can vary to a certain extent, depending upon such factors as the product's intended market segment, its desired nutritional characteristics, flavor profile, and the like. For example, in certain aspects, it can generally be an option to add further ingredients to the formulation of a particular beverage product. For example, additional (i.e., more and/or other) sweeteners can be added, flavorings, electrolytes, vitamins, fruit juices or other fruit products, tastants, masking agents and the like, flavor enhancers, and/or carbonation typically may be added to any such formulations to vary the taste, mouthfeel, nutritional characteristics, etc. In some aspects, the disclosed beverage can be a cola beverage that contains water, a disclosed taste modulator composition, an acidulant, and flavoring. Exemplary flavorings can be, for example, cola flavoring, citrus flavoring, and spice flavorings. In some aspects, carbonation in the form of carbon dioxide can be added for effervescence. In other aspects, preservatives can be added, depending upon the other ingredients, production technique, desired shelf life, etc. In certain aspects, caffeine can be added. In a further aspect, a disclosed beverage can be a cola-flavored carbonated beverage, characteristically containing carbonated water, sweetener, kola nut extract and/or other flavoring, caramel coloring, one or more acids, and optionally other ingredients.

In various aspects, the product can be foods with health claims such as foods with nutrient function claims, nutritional supplements and medical foods.

In various aspects, the product can be in a form in which the consumption unit volume per time is packed or filled in a bag, box, or the like in an amount to be eaten in one meal or in a form in which a beverage, in which the beverage components are filled in a bottle or the like and mixed with a fluid, such as water, in a form to be consumed in one meal.

In one aspect of the present disclosure, a disclosed tabletop bitterness modifying composition is provided a taste modulator composition. The tabletop composition optionally can further include at least one bulking agent, additive, anti-caking agent, functional ingredient and combinations thereof. The disclosed tabletop bitterness modifying composition can be present in the form of a solid or a liquid. The disclosed liquid tabletop bitterness modifying composition can comprise water and/or, other liquid carrier, and optionally additives, such, as for example polyols (e.g. erythritol, sorbitol, propylene glycol or glycerol), acids (e.g. citric acid), antimicrobial agents (e.g. benzoic acid or a salt thereof).

In another aspect of the present disclosure, a disclosed tabletop bitterness modifying composition is provided that includes a bulking material and a taste modulator composition In still another aspect, the disclosed tabletop bitterness modifying composition may include optional ingredients such as for example, characterizing flavors and colors. Alternatively, optional ingredients may be added to the taste modulator composition. It is also possible that optional ingredients may be added to both the disclosed tabletop bitterness modifying composition and the taste modulator composition. Such optional ingredients generally are known to those of skill in the art and may include, for instance, coloring agents, carriers, flavor compounds and the like. For instance, the taste modulator composition may include a strawberry flavor compound to provide a sweetener composition capable of delivering not only a sweet flavor but also a strawberry flavor. This could then be incorporated into a strawberry yogurt product to increase the perception of strawberry flavor compared to a yogurt product without the taste modulator composition. Alternatively, a disclosed tabletop bitterness modifying composition may be colored to a golden brown color to simulate the appearance of raw sugar. Other optional ingredients may include certain carriers and inactive ingredients. These carriers and inactive ingredients may merely facilitate processing of the sweetener. Additionally, a flow agent or anti-caking agent such as tricalcium phosphate may be added to improve flowability of a tabletop sweetener.

A disclosed tabletop bitterness modifying composition may take many forms including, but not limited to, a crystal, a powder, a tablet, a cube, a glaze or coating, a granulated product, or combinations thereof.

In some cases, a disclosed tabletop bitterness modifying composition may be provided in the form of a crystal that has an appearance comparable to that of sucrose crystals, e.g., to improve end user acceptance of the sweetener compositions. It may also be desirable to provide the sweetener in the form of a crystal that has similar solubility profile to sucrose, which becomes apparent, e.g., when the sweetener is mixed into an unsweetened beverage.

In some circumstances, the disclosed tabletop bitterness modifying composition is not formulated to mimic the appearance or solubility characteristics of sucrose, is may be formulated to minimize volume, maximize solubility, maximize stability, or otherwise improve product handling and distribution.

One form of a disclosed tabletop bitterness modifying composition may be an admixture. The disclosed tabletop bitterness modifying composition may also be provided in the form of coated granules in which one or more first component of the sweetener composition is coated over one or more second component of the sweetener composition. For example, the taste modulator composition may be coated onto granules, crystals, or other forms of a flavorant or flavor, such that taste buds are first exposed to the taste modulator composition, and then to the flavorant or flavor. In this manner, the taste buds are modified by the taste modulator composition in preparation for exposure to the flavorant or flavor. In another example, the flavorant or flavor may be coated onto granules, crystals, or other forms of the taste modulator composition, such that taste buds are first exposed to the flavorant or flavor, followed by exposure to the taste modulator composition, which alters the perceived sweetness of the flavorant or flavor. This arrangement allows the taste modulator composition to potentially mask a bitter aftertaste associated with a flavorant or flavor while minimally affecting its initial perception of sweetness. In yet another example, the flavorant or flavor and taste modulator composition may be coated onto granules, crystals, or other forms of a bulking material, such that taste buds are first exposed to the flavorant or flavor and taste modulator composition, followed by exposure to the bulking material.

The disclosed tabletop bitterness modifying composition may further include one or more bulking or filling materials. In one aspect of the present disclosure, the bulking material may add bulk to the sweetener thereby making a single serving of the present compositions more similar to that of sucrose. End users of a sweetener may also find it easier to control the amount of sweetener added to a food or beverage, particularly when the serving size is similar to a known sweetener. Bulking materials may also contribute to body, viscosity, and other aspects of mouthfeel in liquids; volume, cell structure, crumb structure, and humectancy in baked goods; control over the freezing and melting points of foods and beverages; and overall visual and textural impressions of foods and beverages that include the present sweetener. In a further aspect, the bulking material itself may contribute to an increased sweet quality of the flavorant or flavor. In another aspect, the bulking material is low to non-caloric and may provide less than about 0.2 calories per gram of bulking agent.

In still another aspect of the present disclosure, the bulking material has a uniform crystalline structure, i.e. narrow particle size distribution. The uniform crystalline structure may provide for greater control over the ratio of bulking material to flavorant or flavor to taste modulator composition. In one aspect of the present disclosure, the bulking material has a size of from about 0.125 mm to about 1.0 mm. In another aspect of the present disclosure, the bulking material has a size of from about 0.21 mm to about 0.71 mm. In still another aspect of the present disclosure, the bulking material has a size of from about 0.25 mm to about 0.60 mm.

In yet another aspect of the present disclosure, the bulking agent has a solubility profile that is slower than either the flavorant or flavor or taste modulator composition. Thus, if the flavorant or flavor and taste modulator composition were to be deposited onto a bulking agent to form a tabletop sweetener product, the tabletop sweetener product may actually perform more like sugar when introduced into a beverage, particularly a cold beverage, where the granules do not immediately dissolve.

Exemplary bulking materials may be selected from the group consisting of maltodextrin, corn syrup solids, sucrose, fructose, glucose, invert sugar, sorbitol, xylose, ribulose, mannose, xylitol, mannitol, galactitol, erythritol, maltitol, lactitol, isomalt, maltose, tagatose, lactose, inulin, glycerol, propylene glycol, polyols, polydextrose, fructooligosaccharides, cellulose and cellulose derivatives, trehalose, isomaltulose, arabinogalactan, gum Arabic, gum tragacanth, guar gum and hydrolyzed guar gum, and mixtures thereof. It may also be possible to utilize certain starches and modified starches.

In one aspect of the present disclosure, the bulking material is erythritol. In another aspect, the bulking material is glycerol or propylene glycol. These particular bulking materials are available in a liquid form, which may provide for a disclosed liquid tabletop bitterness modifying composition preparation.

Exemplary filling agents include, but are not limited to, filling agents such as cellulose and its derivatives including crystal cellulose, hydroxypropyl cellulose; starch and its derivatives including wheat starch, corn starch, sodium carboxymethyl starch, and dextrin; natural high-polymer compounds such as gum arabic and sodium alginate; sugars and their derivatives including glucose, maltose, sorbitol, maltitol, and mannitol; and inorganic salts including sodium chloride, calcium carbonate, and magnesium silicate; binding agents such as guar gum, synthetic aluminum silicate, stearic acid, highly-polymer polyvinylpyrrolidone, and lactic acid; lubricants such as talc, magnesium stearate, and polyethylene glycol 6000; disintegrating agents such as adipic acid, calcium stearate, and white sugar; surface acting agents such as sucrose fatty acid esters, soybean lecithin, polyoxyethylene hydrogenated castor oil, and polyoxyethylene monostearate esters; thickening agents such as sodium carboxymethyl cellulose, carboxyvinyl polymers, xanthan gum, and gelatin; coating agents such as ethyl acrylate methyl methacrylate copolymer dispersion, caramel, carnauba wax, shellac, white sugar, and pullulan; pH adjusting agents such as citric acid, sodium citrate, acetic acid, sodium acetate, and sodium hydroxide; antioxidant agents such as ascorbic acid, tocopherol acetate, natural vitamin E, and propyl gallate; flavorings such as aspartame, licorice extract, and saccharin; preservatives, such as sodium benzoate, sodium edetate, sorbic acid, sodium sorbate, methyl parahydroxybenzoate, and butyl parahydroxybenzoate; and coloring agents such as red iron oxide, yellow iron oxide, black iron oxide, carmine, Food Blue No. 1, Food Yellow No. 4, Food Yellow No. 4 Aluminum Lake, Food Yellow No. 2, and sodium copper chlorophyllin.

The present disclosure further includes methods of preparing the disclosed tabletop bitterness modifying composition. In one aspect, the disclosed tabletop bitterness modifying composition of the present disclosure is prepared by dissolving a flavorant or flavor and a taste modulator composition in water. The flavorant or flavor and taste modulator composition may either be dissolved individually to form two aqueous solutions or in combination to form a single aqueous solution containing both the flavorant or flavor and taste modulator composition. In the event that the flavorant or flavor and taste modulator composition are dissolved in combination, the flavorant or flavor and taste modulator composition may be added in any order, including simultaneously. In the event the flavorant or flavor and taste modulator composition are dissolved individually, they may be later combined into a single, aqueous mixture. In one aspect, the temperature of the water is at room temperature. In another aspect, the temperature of the water is heated, such as for example to from about 10 to about 70° C. In still another aspect, the temperature of the water used for the flavorant or flavor is heated while the temperature of the water used for the taste modulator composition is at room temperature. Where the taste modulator composition includes more than one component, such as for example, a plurality of congruent flavor volatiles and at least one non-congruent flavor volatile, the components may be delivered in a single fraction or in more than one fraction. For example, the components may be added using a powder fraction containing a blend of dry powdered components and a liquid fraction containing and blend of the remaining components that have been dissolved in an appropriate carrier solution such as water and ethanol. The sweetener can then be processed in a number of ways, such as for example, spray drying, to reduce the moisture level of the sweetener.

In another aspect, is provided a method of preparing a disclosed tabletop bitterness modifying composition. Generally, a flavorant or flavor and a taste modulator composition are deposited onto a bulking material having a size distribution of from about 0.125 mm to about 1.0 mm. The flavorant or flavor and taste modulator composition can be deposited in any order, including simultaneously. Methods to deposit the flavorant or flavor and taste modulator composition will be generally known to one of skill in the art. For example, the bulking material can be placed in a coating vessel (positioned on the right side of the diagram) and air is blown through the vessel (from the bottom of the vessel through the top) in order to cause the bulking material to move about randomly inside of the vessel (i.e., the particles are fluidized). Next, a solution comprising flavorant or flavor in water is introduced into the vessel and is allowed to deposit on the surface of the bulking material. Heated air is blown through the coating vessel in order to dry the flavorant or flavor onto the bulking material. After coating the bulking material with flavorant or flavor, the taste modulator composition is introduced into the coating vessel as a water-based solution. Similar to the flavorant or flavor, the taste modulator composition deposits on the surface of the bulking material and is dried by blowing air through the coating vessel. In a particular aspect of the present disclosure, the air that is blown through the coating vessel is not heated. This may reduce thermal degradation of the taste modulator composition. The resulting disclosed tabletop bitterness modifying composition includes a bulking material with flavorant or flavor and a taste modulator composition deposited on its surface. The resulting tabletop sweetener may also be prepared by first introducing the taste modulator composition into the vessel and then introducing the flavorant or flavor solution into the vessel. Alternatively, the flavorant or flavor solution and liquid taste modulator composition may be added simultaneously into the vessel. In one aspect, the flavorant or flavor is rebaudioside A and the bulking material is erythritol. In another aspect, the size distribution of the bulking material is such that the tabletop sweetener has a desired taste and serving-to-serving consistency. In particular, the size distribution of the bulking material is selected to provide tabletop sweetener particles that have the desired ratio of flavorant or flavor to bulking material and flavorant or flavor to taste modulator composition. Furthermore, the tabletop sweetener particle size is similar to sugar.

In still another aspect, an flavorant or flavor is dissolved in room temperature water. A taste modulator composition, also at room temperature, is blended into the flavorant or flavor-water mixture. A bulking material is added to an agglomeration unit where it is suspended by heated air. While suspended, the mixture of flavorant or flavor, taste modulator composition and water is sprayed into the agglomeration unit in such a way as to allow the components to deposit onto the bulking material. By controlling the temperature in the agglomeration unit the water is removed and the moisture content of tabletop sweetener is comparable to the starting moisture of the bulking material.

In yet another aspect, a flavorant or flavor is dissolved in heated water. In one aspect, the heated water increases the solubility of the flavorant or flavor and therefore less water is necessary to fully dissolve the flavorant or flavor. A bulking material is added to an agglomeration unit where it is suspended by heated air. While suspended, the mixture of flavorant or flavor and water is sprayed into the agglomeration unit in such a way as to allow the components to deposit onto the bulking material. The flavorant or flavor is then dried onto the bulking material using heated air. In one aspect, the air is heated to from about 20 to about 130° C. In another aspect, the air is heated to from about 60 to about 70° C. The bulking material deposited with flavorant or flavor continues to be suspended by air only the temperature of the air is reduced. In one aspect the temperature of the air is reduced to ambient temperature. A mixture of a taste modulator composition and room temperature water is then introduced into the agglomeration unit in such a way as to allow the components to deposit onto the bulking material and flavorant or flavor. By controlling the temperature in the agglomeration unit the water is removed to obtain final moisture content of the tabletop sweetener comparable to that of the starting moisture of the bulking material. In one aspect, the air temperature is from about 20 to about 130° C. In an aspect, the method produces a tabletop sweetener in which the ratio on a dry weight basis of bulking material to flavorant or flavor to taste modulator composition is from about 225:1.80:1 to about 14,370:115:1. In a further aspect, the bulking material is erythritol and the flavorant or flavor is rebaudioside A.

Also provided in the present disclosure are food and beverage products containing either the disclosed tabletop bitterness modifying composition. Exemplary foods and beverages include baked goods, chocolate, candy and confections, chewing gum, ice cream, yogurt, breakfast cereal, oatmeal, pudding, fruit preserves and preparations, breakfast bars, protein bars, granola bars, cereal coatings, syrups, marinades, ketchup, salad dressings, baby food, pet food, animal feed, soft drinks, fruit juices, coffee, tea, sport and energy drinks, and other foods and beverages. A particular class of beverages for which the present compositions and methods are useful is diet soft drinks (or sodas), such as colas, citrus and fruit flavored beverages, and the like. Additionally, pharmaceutical and over the counter drug products may contain either the disclosed tabletop bitterness modifying composition.

### Medicaments Comprising the Disclosed Taste Modulator Compositions

In various aspects, the present disclosure relates to a medicament, e.g., a medicament comprising a pharmaceutical compositions comprising a therapeutically effective amount of at least one therapeutic agent, or a pharmaceutically acceptable salt thereof, and a pharmaceutically acceptable carrier. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one therapeutic agent, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral administration.

In accordance with the present disclosure, a medicament, such as a medicament comprising a pharmaceutical composition for oral use, may be associated with bitter taste, even if transiently in contact with mouth. Patients frequently will exhibit poor compliance with dosing regimens if they experience unpleasant effects with administration of a medicament, such as a bitter taste. In a further aspect, the medicaments disclosed herein comprise a taste modulator composition in an amount sufficient to mitigate, modulate, or lower the bitter taste of the medicament. In a still further aspect, the baseline medicament, i.e., the medicament exhibiting an undesirable bitter taste, is modified such that some or all of the pharmaceutically acceptable carrier is replaced with a disclosed taste modulator composition on a one-for-one weight basis. In a still further aspect, a percentage of the pharmaceutically acceptable carrier is replaced on a weight basis, with a disclosed taste modulator composition selected from about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%; or a range having as an lower and upper limit any two numbers selected from the foregoing; or any subset of the foregoing numbers.

In other aspects, the baseline medicament, i.e., the medicament exhibiting an undesirable bitter taste, is modified such that the pharmaceutically acceptable carrier is supplemented with a disclosed taste modulator composition, e.g., on a weight basis, by addition of a taste modulator composition that is a percentage fraction of the pharmaceutically carrier. In a still further aspect, the pharmaceutically acceptable carrier is supplemented by addition of a taste modulator composition in an amount that is a percentage of the pharmaceutically acceptable carrier selected from about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%; or a range having as an lower and upper limit any two numbers selected from the foregoing; or any subset of the foregoing numbers.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₃H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and dibutrylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulphoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

### Sensory Testing Methodology

In various aspects, the present disclosure pertains to methods of testing the disclosed taste modulator compositions, e.g., a sensory methodology or method for sweetener evaluation. In a particular aspect, the sensory methodology used is known as a “Flavor Profile Analysis” as has been previously described (see: B. T. Carr, S. D. Pecore, K. M. Gibes and G. E. DuBois, “Sensory Methods for Sweetener Evaluation”, Chapter 11 In Flavor Measurement, C. T. Ho and C. H. Manley, Eds., Marcel Dekker, New York, N.Y., 1993). The full set of sensory parameters that are assessed in the sensory testing method are: (a) Sweetness Intensity (which can be abbreviated in tables herein as “S”); (b) Sourness Intensity ((which can be abbreviated in tables herein as “So”); (c) Saltiness Intensity (which can be abbreviated in tables herein as “Sa”); (d) Bitterness Intensity (which can be abbreviated in tables herein as “B”); (e) Body/Mouthfeel Intensity (which can be abbreviated in tables herein as “B/MF”); (f) Astringency Intensity (which can be abbreviated in tables herein as “A”); (g) Sweetness Linger (which can be abbreviated in tables herein as “SL”); (h) Sweetness Appearance Time (which can be abbreviated in tables herein as “AT”); and (i) Sweetness Desensitization (which can be abbreviated in tables herein as “SD”). In conducting the sensory testing method, both individual and group scores can be averaged and subjected to further statistical analysis.

Typically, a panel of 15 subjects is recruited from a larger group based on a panelist's ability to correctly rank a series of 6, 7, 8, 9 and 10% sucrose solutions in order of increasing sweetness intensity. The panelists are then trained to gain familiarity with the following taste attributes based on the tastes of the stimuli shown in parentheses with the taste attribute: (a) Sweet (Sucrose); (b) Sour (Citric Acid); (c) Salty (Sodium Chloride); (d) Bitter (Caffeine); (e) Body/Mouthfeel (Glucose); and (f) Astringency (Alum). Following familiarization with foregoing six taste attributes, panelists are trained in the technique of intensity scaling with a range of sucrose solutions (2.5, 5, 7.5, 10, 12.5 and 15% sucrose). In the training program, panelists were provided these sucrose standard solutions for the purpose of memorization of their perceived intensities on a 0-15 scale. Panelists are then provided sucrose solutions as unknowns and required to correctly rate their intensities on a 0-15 scale. In the next stage of training, panelists are instructed to rate the intensities of sour (citric acid), salty (sodium chloride), bitter (caffeine), body/mouthfeel (glucose) and astringent (alum) samples based on the learned 0-15 sucrose intensity scale.

In addition to rating the intensities of the foregoing six attributes, panelists are also trained in the rating of sweetness onset time, sweetness linger and sweetness desensitization. Each of these briefly discussed in the following.

Sweetness Appearance Time (AT): This time begins when the sample is taken into the mouth and is until maximal sweetness intensity is perceived; Permissible Ratings are: (i) R (Rapid as for 10% Sucrose), and in scoring, R is assigned a numerical score of 0.0; (ii) D (Delay as for 500 PPM REBA), and in scoring, D is assigned a numerical score of 2.5; and (iii) SD (Significant Delay as for 3000 PPM Mono Ammonium Glycyrrhizinate), and in scoring, SD is assigned a numerical score of 5.0.

Sweetness Linger (SL): On tasting the sample and quantifying Attributes a-g, the sample is expectorated @ 15 sec and the mouth rinsed with water (1×15 mL) and the water expectorated @ 30 sec. The tongue and mouth are then held motionless and attention is paid to sweetness buildup in the mouth over the next 120 sec. The perceived sweetness intensity @ 120 sec is SL. 10% Sucrose and 500 PPM REBA, in water, are standards and exhibit SLs of 0 and 5 under these conditions.

Sweetness Desensitization (SD): At the end of the SL scaling, the level of desensitization or tongue-numbing perceived is rated. Permissible Ratings are: (i) N (None as for 10% Sucrose), and in scoring, N is assigned a numerical score of 0.0; (ii) S (Slight as for 500 PPM REBA), and in scoring, S is assigned a numerical score of 2.5; and (iii) M (Medium as for 1000 PPM REBA), and in scoring, M is assigned a numerical score of 5.0.

### ASPECTS

In various aspects, the present disclosure relates to the exemplary aspects provided herein below.

Aspect 1. A composition comprising: a taste modulator component comprising a first salt having a first cation selected from Na⁺, K⁺, Ca²⁺, and Mg²⁺.

Aspect 2. The composition of Aspect 1, wherein the first salt has a first anion comprising gluconate (CH₁₁O₇⁻), citrate (C₆H₅O₇⁻³), hydrogen citrate (C₆H₆O₇⁻²), dihydrogen citrate (C₆H₇O₇⁻), malate (C₄H₆O₅²−), hydrogen malate (C₄H₇O₅⁻), maleate (C₄H₂O₄⁻²), hydrogen maleate (C₄H₃O₄⁻), fumarate (C₄H₂O₄⁻²), hydrogen fumarate (C₄H₃O₄⁻), succinate (C₄H₄O₄⁻²), hydrogen succinate (C₄H₅O₄⁻), glutarate (C₅H₆O₄⁻²), hydrogen glutarate (C₅H₇O₄⁻), adipate (C₆H₈O₄⁻²), hydrogen adipate (C₆H₉O₄⁻), lactate (C₃H₅O₃⁻), tartrate (C₄H₄O₄⁻²), bitartrate (C₄H₅O₆⁻), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻), nitrate (NO₃⁻), carbonate (CO₃⁻²), bicarbonate (HCO₃⁻), glycerate (C₃H₅O₄⁻), glycolate (C₂H₃O₃⁻), or combinations thereof.

Aspect 3. The composition of Aspect 2, where the first anion comprises citrate (C₆H₅O₇⁻³), chloride (Cl⁻), phosphate (PO₄⁻³), carbonate (CO₃⁻²), sulfate (SO₄⁻²), bisulfate, (HSO₄) and combinations thereof.

Aspect 4. The composition of Aspect 2, where the first anion comprises citrate (C₆H₅O₇⁻³).

Aspect 5. The composition of Aspect 2, where the first anion comprises chloride (Cl⁻).

Aspect 6. The composition of any one of Aspect 1-Aspect 5, wherein the taste modulator component further comprises a second salt having a second cation selected from Na⁺, K⁺, Ca²⁺ and Mg²⁺; and wherein the first cation and the second cation are not the same.

Aspect 7. The composition of Aspect 6, wherein the second salt has a second anion comprising gluconate (C₆H₁₁O₇⁻), citrate (C₆H₅O₇⁻³), hydrogen citrate (C₆H₆O₇⁻²), dihydrogen citrate (C₆H₇O₇⁻), malate (C₄H₆O₅⁻²), hydrogen malate (C₄H₇O₅⁻), maleate (C₄H₂O₄⁻²), hydrogen maleate (C₄H₃O₄⁻), fumarate (C₄H₂O₄⁻²), hydrogen fumarate (C₄H₃O₄⁻), succinate (C₄H₄O₄⁻²), hydrogen succinate (C₄H₅O₄⁻), glutarate (C₅H₆O₄⁻²), hydrogen glutarate (C₅H₇O₄⁻), adipate (C₆H₈O₄⁻²), hydrogen adipate (C₆H₉O₄⁻), lactate (C₃H₅O₃⁻), tartrate (C₄H₄O₅⁻²), bitartrate (C₄H₅O₆⁻), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻), nitrate (NO₃⁻), carbonate (CO₃⁻²), bicarbonate (HCO₃⁻), glycerate (C₃H₅O₄⁻), glycolate (C₂H₃O₃⁻), or combinations thereof.

Aspect 8. The composition of Aspect 7, where the second anion comprises citrate (C₆H₅O₇⁻³), chloride (Cl⁻), phosphate (PO₄⁻³), carbonate (CO₃⁻²), sulfate (SO₄⁻²), bisulfate, (HSO₄⁻) and combinations thereof.

Aspect 9. The composition of Aspect 7, where the second anion comprises citrate (C₆H₅O₇⁻³).

Aspect 10. The composition of Aspect 7, where the second anion comprises chloride (Cl⁻).

Aspect 11. The composition of any one of Aspect 1-Aspect 10, wherein the taste modulator component further comprises a third salt having a third cation selected from Na⁺, K⁺, Ca²⁺, and Mg²⁺; and wherein the first cation, the second cation, and the third cation are not the same.

Aspect 12. The composition of Aspect 11, wherein the third salt has a third anion comprising gluconate (C₆H₁₁O₇⁻), citrate (C₆H₅O₇⁻³), hydrogen citrate (C₆H₆O₇⁻²), dihydrogen citrate (C₆H₇O₇⁻), malate (C₄H₆O₅⁻²), hydrogen malate (C₄H₇O₅⁻), maleate (C₄H₂O₄⁻²), hydrogen maleate (C₄H₃O₄⁻), fumarate (C₄H₂O₄⁻²), hydrogen fumarate (C₄H₃O₄⁻), succinate (C₄H₄O₄⁻²), hydrogen succinate (C₄H₅O₄⁻), glutarate (C₅H₆O₄⁻²), hydrogen glutarate (C₅H₇O₄⁻), adipate (C₆H₈O₄⁻²), hydrogen adipate (C₆H₉O₄⁻), lactate (C₃H₅O₃⁻), tartrate (C₄H₄O₅⁻²), bitartrate (C₄H₅O₆⁻), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻), nitrate (NO₃⁻), carbonate (CO₃⁻²), bicarbonate (HCO₃⁻), glycerate (C₃H₅O₄⁻), glycolate (C₂H₃O₃⁻), or combinations thereof.

Aspect 13. The composition of Aspect 12, where the third anion comprises citrate (C₆H₅O₇⁻³), chloride (Cl⁻), phosphate (PO₄⁻³), carbonate (CO₃⁻²), sulfate (SO₄⁻²), bisulfate, (HSO₄) and combinations thereof.

Aspect 14. The composition of Aspect 12, where the third anion comprises citrate (C₆H₅O₇⁻³).

Aspect 15. The composition of Aspect 12, where the third anion comprises chloride (Cl⁻).

Aspect 16. The composition of any one of Aspect 1-Aspect 15, wherein the taste modulator component further comprises a fourth salt having a fourth cation selected from Na⁺, K⁺, Ca²⁺, and Mg²⁺; and wherein the first cation, the second cation, the third cation, and the fourth cation are not the same.

Aspect 17. The composition of Aspect 16, wherein the fourth salt has a fourth anion comprising gluconate (C₆H₁₁O₇), citrate (C₆H₅O₇⁻³), hydrogen citrate (C₆H₆O₇⁻²), dihydrogen citrate (C₆H₇O₇⁻), malate (C₄H₆O₅⁻²), hydrogen malate (C₄H₇O₅⁻), maleate (C₄H₂O₄⁻²), hydrogen maleate (C₄H₃O₄⁻), fumarate (C₄H₂O₄⁻²), hydrogen fumarate (C₄H₃O₄⁻), succinate (C₄H₄O₄⁻²), hydrogen succinate (C₄H₅O₄⁻), glutarate (C₅H₆O₄⁻²), hydrogen glutarate (C₅H₇O₄⁻), adipate (C₆H₈O₄⁻²), hydrogen adipate (C₆H₉O₄⁻), lactate (C₃H₅O₃⁻), tartrate (C₄H₄O₆⁻²), bitartrate (C₄H₅O₆⁻), phosphate (PO₄⁻³), monohydrogen phosphate (HPO₄⁻²), dihydrogen phosphate (H₂PO₄⁻), fluoride (F⁻), chloride (Cl⁻), sulfate (SO₄⁻²), bisulfate (HSO₄⁻), nitrate (NO₃⁻), carbonate (CO₃⁻²), bicarbonate (HCO₃⁻), glycerate (C₃H₅O₄⁻), glycolate (C₂H₃O₃⁻), or combinations thereof.

Aspect 18. The composition of Aspect 17, where the fourth anion comprises citrate (C₆H₅O₇⁻³), chloride (Cl⁻), phosphate (PO₄⁻³), carbonate (CO₃⁻²), sulfate (SO₄⁻²), bisulfate, (HSO₄⁻) and combinations thereof.

Aspect 19. The composition of Aspect 17, where the fourth anion comprises citrate (C₆H₅O₇⁻³).

Aspect 20. The composition of Aspect 17, where the fourth anion comprises chloride (Cl⁻).

Aspect 21. The composition of any one of Aspect 1-Aspect 20, further comprising a CaSR modulator agonist, CaSR positive allosteric modulator, or combinations thereof.

Aspect 22. The composition of Aspect 21, wherein the CaSR modulator is a CaSR agonist, CaSR positive allosteric modulator, or combinations thereof.

Aspect 23. The composition of Aspect 21, wherein the CaSR modulator is a polyamine, aminoglycoside antibiotic, a salt, an amino acid, a peptide, other CaSR modulator, or combinations thereof.

Aspect 24. The composition of Aspect 23, wherein the amino acid is an L-amino acid.

Aspect 25. The composition of Aspect 24, wherein the amino acid is glycine, leucine, glutamate, aspartate, tryptophan, phenylalanine, or combinations thereof.

Aspect 26. The composition of Aspect 23, wherein the polyamine is putrescine, cadaverine, spermidine, spermine, poly-L-arginine, or combinations thereof.

Aspect 27. The composition of Aspect 23, wherein the aminoglycoside antibiotic is amikacin, gentamicin, kanamycin, neomycin, netilmicin, paromomycin, streptomycin, tobramycin, apramycin, or combinations thereof.

Aspect 28. The composition of Aspect 23, wherein the salt is an aluminum salt, an iron salt, a gadolinium salt, a zinc salt, a strontium salt.

Aspect 29. The composition of Aspect 23, wherein the peptide has 2-8 amino acids.

Aspect 30. The composition of Aspect 29, wherein the peptide has a γ-glutamyl residue.

Aspect 31. The composition of Aspect 29 or Aspect 30, wherein the peptide is Glu-Asp, Glu-Glu, Asp-Glu, Thr-Glu, Asp-Glu-Ser, Glu-Gly-Ser, Glu-Val-Glu, Gly-Leu-Pro-Asp, Gly-His-Gly-Asp, Asp-Asp-Asp-Asp, Gly-Asp-Glu-Glu-Ser-Leu-Ala, or combinations thereof.

Aspect 32. The composition of Aspect 29, wherein the peptide is glutathione.

Aspect 33. The composition of Aspect 23, wherein the other CaSR modulator is cinacalcet, calindol, or a combination thereof.

Aspect 81. The composition of any one of Aspect 1-Aspect 33, wherein the composition in a beverage, food, nutraceutical, pharmaceutical, or cosmetic is from about 0.1 mM to about 30 mM; and wherein the total cation represents a sum of the first cation, and when present, the second cation, the third cation, and the fourth cation.

Aspect 82. The composition of Aspect 82, wherein the total cation is present in an effective amount to provide a concentration when present in a beverage, food, nutraceutical, pharmaceutical, or cosmetic from about 0.1 mM to about 10 mM Na⁺, when Na⁺ is present; from about 0.1 mM to about 20 mM K⁺, when K⁺ is present; from about 0.1 mM to about 5 mM Mg+, when Mg+ is present; from about 0.1 mM to about 5 mM Ca²⁺, when Ca²⁺ is present; and combinations thereof provided that the total cation present is from about 0.1 mM to about 50 mM.

Aspect 83. The composition of Aspect 82, wherein the total cation is present in an effective amount to provide a concentration when present in a beverage, food, nutraceutical, pharmaceutical, or cosmetic from about 0.1 mM to about 40 mM.

Aspect 84. The composition of Aspect 82, wherein the total cation is present in an effective amount to provide a concentration when present in a beverage, food, nutraceutical, pharmaceutical, or cosmetic from about 0.1 mM to about 30 mM.

Aspect 85. The composition of Aspect 82, wherein the total cation is present in an effective amount to provide a concentration when present in a beverage, food, nutraceutical, pharmaceutical, or cosmetic from about 0.1 mM to about 20 mM.

Aspect 86. The composition of Aspect 82, wherein the total cation is present in an effective amount to provide a concentration when present in a beverage, food, nutraceutical, pharmaceutical, or cosmetic from about 0.1 mM to about 10 mM.

Aspect 87. The composition of Aspect 82, wherein the total cation is present in an effective amount to provide a concentration when present in a beverage, food, nutraceutical, pharmaceutical, or cosmetic from about 1 mM to about 25 mM Na⁺.

Aspect 88. The composition of Aspect 87, wherein the total cation is present in an effective amount to provide a concentration when present in a beverage, food, nutraceutical, pharmaceutical, or cosmetic from about 1 mM to about 17.5 mM Na⁺.

Aspect 89. The composition of Aspect 87, wherein the total cation is present in an effective amount to provide a concentration when present in a beverage, food, nutraceutical, pharmaceutical, or cosmetic from about 1 mM to about 10 mM Na⁺.

Aspect 90. The composition of Aspect 87, wherein the total cation is present in an effective amount to provide a concentration when present in a beverage, food, nutraceutical, pharmaceutical, or cosmetic from about 5 mM to about 10 mM Na⁺.

Aspect 91. The composition of Aspect 82, wherein the total cation is present in an effective amount to provide a concentration when present in a beverage, food, nutraceutical, pharmaceutical, or cosmetic from about 1 mM to about 25 mM K⁺.

Aspect 92. The composition of Aspect 91, wherein the total cation is present in an effective amount to provide a concentration when present in a beverage, food, nutraceutical, pharmaceutical, or cosmetic from about 1 mM to about 17.5 mM K⁺.

Aspect 93. The composition of Aspect 91, wherein the total cation is present in an effective amount to provide a concentration when present in a beverage, food, nutraceutical, pharmaceutical, or cosmetic from about 1 mM to about 10 mM K⁺.

Aspect 94. The composition of Aspect 91, wherein the total cation is present in an effective amount to provide a concentration when present in a beverage, food, nutraceutical, pharmaceutical, or cosmetic from about 5 mM to about 10 mM K⁺.

Aspect 95. The composition of Aspect 82, wherein the total cation is present in an effective amount to provide a concentration when present in a beverage, food, nutraceutical, pharmaceutical, or cosmetic from about 1 mM to about 15 mM Mg²⁺.

Aspect 96. The composition of Aspect 95, wherein the total cation is present in an effective amount to provide a concentration when present in a beverage, food, nutraceutical, pharmaceutical, or cosmetic from about 1 mM to about 10 mM Mg²⁺.

Aspect 97. The composition of Aspect 95, wherein the total cation is present in an effective amount to provide a concentration when present in a beverage, food, nutraceutical, pharmaceutical, or cosmetic from about 3 mM to about 5 mM Mg²⁺.

Aspect 98. The composition of Aspect 95, wherein the total cation is present in an effective amount to provide a concentration when present in a beverage, food, nutraceutical, pharmaceutical, or cosmetic from about 1 mM to about 5 mM Mg²⁺.

Aspect 99. The composition of Aspect 82, wherein the total cation is present in an effective amount to provide a concentration when present in a beverage, food, nutraceutical, pharmaceutical, or cosmetic from about 1 mM to about 25 mM Ca²⁺.

Aspect 100. The composition of Aspect 99, wherein the total cation is present in an effective amount to provide a concentration when present in a beverage, food, nutraceutical, pharmaceutical, or cosmetic from about 1 mM to about 17.5 mM Ca²⁺.

Aspect 101. The composition of Aspect 99, wherein the total cation is present in an effective amount to provide a concentration when present in a beverage, food, nutraceutical, pharmaceutical, or cosmetic from about 1 mM to about 10 mM Ca²⁺.

Aspect 102. The composition of Aspect 99, wherein the total cation is present in an effective amount to provide a concentration when present in a beverage, food, nutraceutical, pharmaceutical, or cosmetic from about 3 mM to about 6 mM Ca²⁺.

Aspect 103. The composition of Aspect 99, wherein the total cation is present in an effective amount to provide a concentration when present in a beverage, food, nutraceutical, pharmaceutical, or cosmetic from about 1 mM to about 5 mM Ca²⁺.

Aspect 104. The composition of Aspect 82, wherein the total cation is present in an effective amount to provide a concentration when present in a beverage, food, nutraceutical, pharmaceutical, or cosmetic from about 1 mM to about 10 mM Na⁺; and from about 1 mM to about 10 mM K⁺.

Aspect 105. The composition of Aspect 104, wherein the total cation is present in an effective amount to provide a concentration when present in a beverage, food, nutraceutical, pharmaceutical, or cosmetic from about 3 mM to about 7 mM Na⁺; and from about 3 mM to about 7 mM K⁺.

Aspect 106. The composition of Aspect 104, wherein the total cation is present in an effective amount to provide a concentration when present in a beverage, food, nutraceutical, pharmaceutical, or cosmetic from about 4 mM to about 6 mM Na⁺; and from about 4 mM to about 6 mM K⁺.

Aspect 107. The composition of Aspect 82, wherein the total cation is present in an effective amount to provide a concentration when present in a beverage, food, nutraceutical, pharmaceutical, or cosmetic from about 1 mM to about 10 mM Na⁺; and from about 1 mM to about 10 mM Mg²⁺.

Aspect 108. The composition of Aspect 107, wherein the total cation is present in an effective amount to provide a concentration when present in a beverage, food, nutraceutical, pharmaceutical, or cosmetic from about 3 mM to about 7 mM Na⁺; and from about 3 mM to about 7 mM Mg²⁺.

Aspect 109. The composition of Aspect 107, wherein the total cation is present in an effective amount to provide a concentration when present in a beverage, food, nutraceutical, pharmaceutical, or cosmetic from about 4 mM to about 6 mM Na⁺; and from about 4 mM to about 6 mM Mg²⁺.

Aspect 110. The composition of Aspect 82, wherein the total cation is present in an effective amount to provide a concentration when present in a beverage, food, nutraceutical, pharmaceutical, or cosmetic from about 1 mM to about 10 mM Na⁺; and from about 1 mM to about 10 mM Ca²⁺.

Aspect 111. The composition of Aspect 110, wherein the total cation is present in an effective amount to provide a concentration when present in a beverage, food, nutraceutical, pharmaceutical, or cosmetic from about 3 mM to about 7 mM Na⁺; and from about 3 mM to about 7 mM Ca²⁺.

Aspect 112. The composition of Aspect 110, wherein the total cation is present in an effective amount to provide a concentration when present in a beverage, food, nutraceutical, pharmaceutical, or cosmetic from about 4 mM to about 6 mM Na⁺; and from about 4 mM to about 6 mM Ca²⁺.

Aspect 113. The composition of Aspect 82, wherein the total cation is present in an effective amount to provide a concentration when present in a beverage, food, nutraceutical, pharmaceutical, or cosmetic from about 1 mM to about 10 mM K⁺; and from about 1 mM to about 10 mM Mg²⁺.

Aspect 114. The composition of Aspect 113, wherein the total cation is present in an effective amount to provide a concentration when present in a beverage, food, nutraceutical, pharmaceutical, or cosmetic from about 3 mM to about 7 mM K⁺; and from about 3 mM to about 7 mM Mg²⁺.

Aspect 115 The composition of Aspect 113, wherein the total cation is present in an effective amount to provide a concentration when present in a beverage, food, nutraceutical, pharmaceutical, or cosmetic from about 4 mM to about 6 mM K⁺; and from about 4 mM to about 6 mM Mg²⁺.

Aspect 116. The composition of Aspect 82, wherein the total cation is present in an effective amount to provide a concentration when present in a beverage, food, nutraceutical, pharmaceutical, or cosmetic from about 1 mM to about 10 mM K⁺; and from about 1 mM to about 10 mM Ca²⁺.

Aspect 117. The composition of Aspect 116, wherein the total cation is present in an effective amount to provide a concentration when present in a beverage, food, nutraceutical, pharmaceutical, or cosmetic from about 3 mM to about 7 mM K⁺; and from about 3 mM to about 7 mM Ca²⁺.

Aspect 118. The composition of Aspect 116, wherein the total cation is present in an effective amount to provide a concentration when present in a beverage, food, nutraceutical, pharmaceutical, or cosmetic from about 4 mM to about 6 mM K⁺; and from about 4 mM to about 6 mM Ca²⁺.

Aspect 119. The composition of Aspect 82, wherein the total cation is present in an effective amount to provide a concentration when present in a beverage, food, nutraceutical, pharmaceutical, or cosmetic from about 1 mM to about 10 mM Mg²⁺; and from about 1 mM to about 10 mM Ca²⁺.

Aspect 120. The composition of Aspect 119, wherein the total cation is present in an effective amount to provide a concentration when present in a beverage, food, nutraceutical, pharmaceutical, or cosmetic from about 2 mM to about 6 mM Mg²⁺; and from about 2 mM to about 6 mM Ca²⁺.

Aspect 121. The composition of Aspect 119, wherein the total cation is present in an effective amount to provide a concentration when present in a beverage, food, nutraceutical, pharmaceutical, or cosmetic from about 3 mM to about 5 mM Mg²⁺; and from about 3 mM to about 5 mM Ca²⁺.

Aspect 122. The composition of Aspect 82, wherein the total cation is present in an effective amount to provide a concentration when present in a beverage, food, nutraceutical, pharmaceutical, or cosmetic from about 1 mM to about 10 mM Na⁺; from about 1 mM to about 10 mM K⁺; and from about 1 mM to about 10 mM Mg²⁺.

Aspect 123 The composition of Aspect 122, wherein the total cation is present in an effective amount to provide a concentration when present in a beverage, food, nutraceutical, pharmaceutical, or cosmetic of about 1 mM to about 5 mM Na⁺; about 1 mM to about 5 mM K⁺; and about 1 mM to about 5 mM Mg²⁺.

Aspect 124. The composition of Aspect 122, wherein the total cation is present in an effective amount to provide a concentration when present in a beverage, food, nutraceutical, pharmaceutical, or cosmetic from about 2.5 mM to about 5 mM Na⁺; from about 2.5 mM to about 5 mM K⁺; and from about 2.5 mM to about 5 mM Mg²⁺.

Aspect 125. The composition of Aspect 82, wherein the total cation is present in an effective amount to provide a concentration when present in a beverage, food, nutraceutical, pharmaceutical, or cosmetic from about 1 mM to about 10 mM Na⁺; from about 1 mM to about 10 mM K⁺; and from about 1 mM to about 10 mM Ca²⁺.

Aspect 126. The composition of Aspect 125, wherein the total cation is present in an effective amount to provide a concentration when present in a beverage, food, nutraceutical, pharmaceutical, or cosmetic from about 1 mM to about 5 mM Na⁺; from about 1 mM to about 5 mM K⁺; and from about 1 mM to about 5 mM Ca²⁺.

Aspect 127. The composition of Aspect 125, wherein the total cation is present in an effective amount to provide a concentration when present in a beverage, food, nutraceutical, pharmaceutical, or cosmetic from about 2.5 mM to about 5 mM Na⁺; from about 2.5 mM to about 5 mM K⁺; and from about 2.5 mM to about 5 mM Ca²⁺.

Aspect 128. The composition of Aspect 82, wherein the total cation is present in an effective amount to provide a concentration when present in a beverage, food, nutraceutical, pharmaceutical, or cosmetic from about 1 mM to about 10 mM K⁺; from about 1 mM to about 10 mM Mg²⁺; and from about 1 mM to about 10 mM Ca²⁺.

Aspect 129. The composition of Aspect 128, wherein the total cation is present in an effective amount to provide a concentration when present in a beverage, food, nutraceutical, pharmaceutical, or cosmetic from about 1 mM to about 5 mM K⁺; from about 1 mM to about 5 mM Mg²⁺; and from about 1 mM to about 5 mM Ca²⁺.

Aspect 130. The composition of Aspect 128, wherein the total cation is present in an effective amount to provide a concentration when present in a beverage, food, nutraceutical, pharmaceutical, or cosmetic from about 2.5 mM to about 5 mM K⁺; from about 2.5 mM to about 5 mM Mg²⁺; and from about 2.5 mM to about 5 mM Ca²⁺.

Aspect 131. The composition of Aspect 82, wherein the total cation is present in an effective amount to provide a concentration when present in a beverage, food, nutraceutical, pharmaceutical, or cosmetic from about 1 mM to about 10 mM Na⁺; from about 1 mM to about 10 mM K⁺; from about 1 mM to about 10 mM Mg²⁺; and from about 1 mM to about 10 mM Ca²⁺.

Aspect 132. The composition of Aspect 131, wherein the total cation is present in an effective amount to provide a concentration when present in a beverage, food, nutraceutical, pharmaceutical, or cosmetic from about 1 mM to about 4 mM Na⁺; from about 1 mM to about 4 mM K⁺; from about 1 mM to about 4 mM Mg²⁺; and from about 1 mM to about 4 mM Ca²⁺.

Aspect 133. The composition of Aspect 131, wherein the total cation is present in an effective amount to provide a concentration when present in a beverage, food, nutraceutical, pharmaceutical, or cosmetic from about 1 mM to about 3 mM Na⁺; from about 1 mM to about 3 mM K⁺; from about 1 mM to about 3 mM Mg²⁺; and from about 1 mM to about 3 mM Ca²⁺.

Aspect 134 The composition of any one of Aspect 1-Aspect 133, wherein the composition is a powder, a particulate, an agglomerated solid, a solid, a gel, or combinations thereof.

Aspect 135. The composition of Aspect 134, wherein the composition is a powder.

Aspect 136. The composition of Aspect 134, wherein the composition is an agglomerated solid.

Aspect 137. The composition of Aspect 134, wherein the composition is a particulate; and wherein the particulate comprises a nanoparticule, a microparticule, or combinations thereof.

Aspect 138. The composition of any one of 1-Aspect 137, wherein the composition has inhibited bitterness, improved mouthfeel, or a combination thereof, when compared to a baseline; wherein the baseline composition consists essentially of identical components as the composition without the taste modulator component; and wherein the bitterness, mouthfeel, or a combination thereof is determined using a sensory panel study as described herein.

Aspect 139. The composition of Aspect 138, wherein the bitterness of the composition is decreased compared to the baseline composition by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or combinations thereof.

Aspect 140. The composition of Aspect 138, wherein the bitterness of the composition is decreased compared to the baseline composition by about 1.1-fold, about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 2.6-fold, about 2.7-fold, about 2.8-fold, about 2.9-fold, about 3-fold, about 3.1-fold, about 3.2-fold, about 3.3-fold, about 3.4-fold, about 3.5-fold, about 3.6-fold, about 3.7-fold, about 3.8-fold, about 3.9-fold, about 4-fold, about 4.1-fold, about 4.2-fold, about 4.3-fold, about 4.4-fold, about 4.5-fold, about 4.6-fold, about 4.7-fold, about 4.8-fold, about 4.9-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, about 20-fold, about 21-fold, about 22-fold, about 23-fold, about 24-fold, about 25-fold, about 26-fold, about 27-fold, about 28-fold, about 29-fold, about 30-fold, about 31-fold, about 32-fold, about 33-fold, about 34-fold, about 35-fold, about 36-fold, about 37-fold, about 38-fold, about 39-fold, about 40-fold, about 41-fold, about 42-fold, about 43-fold, about 44-fold, about 45-fold, about 46-fold, about 47-fold, about 48-fold, about 49-fold, about 50-fold, about 51-fold, about 52-fold, about 53-fold, about 54-fold, about 55-fold, about 56-fold, about 57-fold, about 58-fold, about 59-fold, about 60-fold, about 61-fold, about 62-fold, about 63-fold, about 64-fold, about 65-fold, about 66-fold, about 67-fold, about 68-fold, about 69-fold, about 70-fold, about 71-fold, about 72-fold, about 73-fold, about 74-fold, about 75-fold, about 76-fold, about 77-fold, about 78-fold, about 79-fold, about 80-fold, about 81-fold, about 82-fold, about 83-fold, about 84-fold, about 85-fold, about 86-fold, about 87-fold, about 88-fold, about 89-fold, about 90-fold, about 91-fold, about 92-fold, about 93-fold, about 94-fold, about 95-fold, about 96-fold, about 97-fold, about 98-fold, about 99-fold, about 100-fold, or combinations thereof.

Aspect 141. The composition of Aspect 138, wherein the mouthfeel of the composition is increased compared to the baseline composition by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%; or combinations thereof.

Aspect 142. The composition of Aspect 138, wherein the mouthfeel of the composition is increased compared to the baseline composition by about 1.1-fold, about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 2.6-fold, about 2.7-fold, about 2.8-fold, about 2.9-fold, about 3-fold, about 3.1-fold, about 3.2-fold, about 3.3-fold, about 3.4-fold, about 3.5-fold, about 3.6-fold, about 3.7-fold, about 3.8-fold, about 3.9-fold, about 4-fold, about 4.1-fold, about 4.2-fold, about 4.3-fold, about 4.4-fold, about 4.5-fold, about 4.6-fold, about 4.7-fold, about 4.8-fold, about 4.9-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, about 20-fold, about 21-fold, about 22-fold, about 23-fold, about 24-fold, about 25-fold, about 26-fold, about 27-fold, about 28-fold, about 29-fold, about 30-fold, about 31-fold, about 32-fold, about 33-fold, about 34-fold, about 35-fold, about 36-fold, about 37-fold, about 38-fold, about 39-fold, about 40-fold, about 41-fold, about 42-fold, about 43-fold, about 44-fold, about 45-fold, about 46-fold, about 47-fold, about 48-fold, about 49-fold, about 50-fold, about 51-fold, about 52-fold, about 53-fold, about 54-fold, about 55-fold, about 56-fold, about 57-fold, about 58-fold, about 59-fold, about 60-fold, about 61-fold, about 62-fold, about 63-fold, about 64-fold, about 65-fold, about 66-fold, about 67-fold, about 68-fold, about 69-fold, about 70-fold, about 71-fold, about 72-fold, about 73-fold, about 74-fold, about 75-fold, about 76-fold, about 77-fold, about 78-fold, about 79-fold, about 80-fold, about 81-fold, about 82-fold, about 83-fold, about 84-fold, about 85-fold, about 86-fold, about 87-fold, about 88-fold, about 89-fold, about 90-fold, about 91-fold, about 92-fold, about 93-fold, about 94-fold, about 95-fold, about 96-fold, about 97-fold, about 98-fold, about 99-fold, about 100-fold; or combinations thereof.

Aspect 143. A method for making the composition of any one of Aspect 1-Aspect 142, comprising: mixing a taste modulator component comprising a first salt having a first cation selected from Na⁺, K⁺, Ca²⁺, and Mg²⁺; wherein the mixing is carried out until a mixture of the taste modulator.

Aspect 144. The method of Aspect 143, wherein mixing is carried out using a rotating drum mixer.

Aspect 145. The method of Aspect 143 or Aspect 144, further comprising mixing a solvent with the taste modulator; and mixing the taste modulator and the solvent until a solution or a suspension is essentially homogeneous.

Aspect 146. The method of Aspect 145, wherein the solvent comprises water.

Aspect 147. The method of Aspect 145 or Aspect 147, further comprising spray-drying the solution or the suspension.

Aspect 148. The method of Aspect 145 or Aspect 147, further comprising lyophilizing the solution or the suspension.

Aspect 149. The method of Aspect 145 or Aspect 147, further comprising tableting the mixture.

Aspect 150. A product comprising the composition of any one of Aspect 1-Aspect 149 or a composition made by the method of any one of Aspect 143-Aspect 149.

Aspect 151. The product of Aspect 150, wherein the product is a beverage, a food, a nutraceutical, or a concentrated savory composition.

Aspect 152. The product of Aspect 151, wherein the product is a beverage.

Aspect 153. The product of any one of Aspect 150-Aspect 152, wherein the product is a carbonated beverage.

Aspect 154. The product of any one of Aspect 150-Aspect 152, wherein the product is a non-carbonated beverage.

Aspect 155. The product of any one of Aspect 150-Aspect 154, wherein the composition comprises from about 0.1 mM to about 30 mM of a total cation; and wherein the total cation represents a sum of the first cation, and when present, the second cation, the third cation, and the fourth cation.

Aspect 156. The product of Aspect 155, wherein the total cation is present in an amount from about 0.1 mM to about 10 mM Na⁺, when Na⁺ is present; from about 0.1 mM to about 20 mM K⁺, when K⁺ is present; from about 0.1 mM to about 5 mM Mg+, when Mg+ is present; from about 0.1 mM to about 5 mM Ca²⁺, when Ca²⁺ is present; and combinations thereof provided that the total cation present is from about 0.1 mM to about 50 mM.

Aspect 157. The product of Aspect 156, wherein the total cation present is from about 0.1 mM to about 40 mM.

Aspect 158. The product of Aspect 156, wherein the total cation present is from about 0.1 mM to about 30 mM.

Aspect 159. The product of Aspect 156, wherein the total cation present is from about 0.1 mM to about 20 mM.

Aspect 160. The product of Aspect 156, wherein the total cation present is from about 0.1 mM to about 10 mM.

Aspect 161. The product of Aspect 156, wherein the total cation present is from about 1 mM to about 25 mM Na⁺.

Aspect 162. The product of Aspect 161, wherein the total cation present is from about 1 mM to about 17.5 mM Na⁺.

Aspect 163. The product of Aspect 161, wherein the total cation present is from about 1 mM to about 10 mM Na⁺.

Aspect 164. The product of Aspect 161, wherein the total cation present is from about 5 mM to about 10 mM Na⁺.

Aspect 165. The product of Aspect 156, wherein the total cation present is from about 1 mM to about 25 mM K⁺.

Aspect 166. The product of Aspect 165, wherein the total cation present is from about 1 mM to about 17.5 mM K⁺.

Aspect 167. The product of Aspect 165, wherein the total cation present is from about 1 mM to about 10 mM K⁺.

Aspect 168. The product of Aspect 165, wherein the total cation present is from about 5 mM to about 10 mM K⁺.

Aspect 169. The product of Aspect 156, wherein the total cation present is from about 1 mM to about 15 mM Mg²⁺.

Aspect 170. The product of Aspect 169, wherein the total cation present is from about 1 mM to about 10 mM Mg²⁺.

Aspect 171. The product of Aspect 169, wherein the total cation present is from about 3 mM to about 5 mM Mg²⁺.

Aspect 172. The product of Aspect 169, wherein the total cation present is from about 1 mM to about 5 mM Mg²⁺.

Aspect 173 The product of Aspect 156, wherein the total cation present is from about 1 mM to about 25 mM Ca²⁺.

Aspect 174. The product of Aspect 173, wherein the total cation present is from about 1 mM to about 17.5 mM Ca²⁺.

Aspect 175. The product of Aspect 173, wherein the total cation present is from about 1 mM to about 10 mM Ca²⁺.

Aspect 176. The product of Aspect 173, wherein the total cation present is from about 3 mM to about 6 mM Ca²⁺.

Aspect 177. The product of Aspect 173, wherein the total cation present is from about 1 mM to about 5 mM Ca²⁺.

Aspect 178. The product of Aspect 156, wherein the total cation present is from about 1 mM to about 10 mM Na⁺; and from about 1 mM to about 10 mM K⁺.

Aspect 179. The product of Aspect 178, wherein the total cation present is from about 3 mM to about 7 mM Na⁺; and from about 3 mM to about 7 mM K⁺.

Aspect 180. The product of Aspect 178, wherein the total cation present is from about 4 mM to about 6 mM Na⁺; and from about 4 mM to about 6 mM K⁺.

Aspect 181. The product of Aspect 156, wherein the total cation present is from about 1 mM to about 10 mM Na⁺; and from about 1 mM to about 10 mM Mg²⁺.

Aspect 182. The product of Aspect 181, wherein the total cation present is from about 3 mM to about 7 mM Na⁺; and from about 3 mM to about 7 mM Mg²⁺.

Aspect 183. The product of Aspect 181, wherein the total cation present is from about 4 mM to about 6 mM Na⁺; and from about 4 mM to about 6 mM Mg²⁺.

Aspect 184. The product of Aspect 156, wherein the total cation present is from about 1 mM to about 10 mM Na⁺; and from about 1 mM to about 10 mM Ca²⁺.

Aspect 185. The product of Aspect 184, wherein the total cation present is from about 3 mM to about 7 mM Na⁺; and from about 3 mM to about 7 mM Ca²⁺.

Aspect 186. The product of Aspect 184, wherein the total cation present is from about 4 mM to about 6 mM Na⁺; and from about 4 mM to about 6 mM Ca²⁺.

Aspect 187. The product of Aspect 156, wherein the total cation present is from about 1 mM to about 10 mM K⁺; and from about 1 mM to about 10 mM Mg²⁺.

Aspect 188. The product of Aspect 187, wherein the total cation present is from about 3 mM to about 7 mM K⁺; and from about 3 mM to about 7 mM Mg²⁺.

Aspect 189. The product of Aspect 1871, wherein the total cation present is from about 4 mM to about 6 mM K⁺; and from about 4 mM to about 6 mM Mg²⁺.

Aspect 190. The product of Aspect 156, wherein the total cation present is from about 1 mM to about 10 mM K⁺; and from about 1 mM to about 10 mM Ca²⁺.

Aspect 191. The product of Aspect 190, wherein the total cation present is from about 3 mM to about 7 mM K⁺; and from about 3 mM to about 7 mM Ca²⁺.

Aspect 192. The product of Aspect 190, wherein the total cation present is from about 4 mM to about 6 mM K⁺; and from about 4 mM to about 6 mM Ca²⁺.

Aspect 193. The product of Aspect 156, wherein the total cation present is from about 1 mM to about 10 mM Mg²⁺; and from about 1 mM to about 10 mM Ca²⁺.

Aspect 194. The product of Aspect 193, wherein the total cation present is from about 2 mM to about 6 mM Mg²⁺; and from about 2 mM to about 6 mM Ca²⁺.

Aspect 195. The product of Aspect 193, wherein the total cation present is from about 3 mM to about 5 mM Mg²⁺; and from about 3 mM to about 5 mM Ca²⁺.

Aspect 196. The product of Aspect 156, wherein the total cation present is from about 1 mM to about 10 mM Na⁺; from about 1 mM to about 10 mM K⁺; and from about 1 mM to about 10 mM Mg²⁺.

Aspect 197. The product of Aspect 196, wherein the total cation present is from about 1 mM to about 5 mM Na⁺; from about 1 mM to about 5 mM K⁺; and from about 1 mM to about 5 mM Mg²⁺.

Aspect 198. The product of Aspect 1960, wherein the total cation present is from about 2.5 mM to about 5 mM Na⁺; from about 2.5 mM to about 5 mM K⁺; and from about 2.5 mM to about 5 mM Mg²⁺.

Aspect 199. The product of Aspect 156, wherein the total cation present is from about 1 mM to about 10 mM Na⁺; from about 1 mM to about 10 mM K⁺; and from about 1 mM to about 10 mM Ca²⁺.

Aspect 200. The product of Aspect 199, wherein the total cation present is from about 1 mM to about 5 mM Na⁺; from about 1 mM to about 5 mM K⁺; and from about 1 mM to about 5 mM Ca²⁺.

Aspect 201. The product of Aspect 199, wherein the total cation present is from about 2.5 mM to about 5 mM Na⁺; from about 2.5 mM to about 5 mM K⁺; and from about 2.5 mM to about 5 mM Ca²⁺.

Aspect 202. The product of Aspect 156, wherein the total cation present is from about 1 mM to about 10 mM K⁺; from about 1 mM to about 10 mM Mg²⁺; and from about 1 mM to about 10 mM Ca²⁺.

Aspect 203. The product of Aspect 202, wherein the total cation present is from about 1 mM to about 5 mM K⁺; from about 1 mM to about 5 mM Mg²⁺; and from about 1 mM to about 5 mM Ca²⁺.

Aspect 204. The product of Aspect 202, wherein the total cation present is from about 2.5 mM to about 5 mM K⁺; from about 2.5 mM to about 5 mM Mg²⁺; and from about 2.5 mM to about 5 mM Ca²⁺.

Aspect 205. The product of Aspect 156, wherein the total cation present is from about 1 mM to about 10 mM Na⁺; from about 1 mM to about 10 mM K⁺; from about 1 mM to about 10 mM Mg²⁺; and from about 1 mM to about 10 mM Ca²⁺.

Aspect 206. The product of Aspect 205, wherein the total cation present is from about 1 mM to about 4 mM Na⁺; from about 1 mM to about 4 mM K⁺; from about 1 mM to about 4 mM Mg²⁺; and from about 1 mM to about 4 mM Ca²⁺.

Aspect 207. The product of Aspect 205, wherein the total cation present is from about 1 mM to about 3 mM Na⁺; from about 1 mM to about 3 mM K⁺; from about 1 mM to about 3 mM Mg²⁺; and from about 1 mM to about 3 mM Ca²⁺.

Aspect 208. A product comprising the composition of any one of Aspect 1-Aspect 149 or a composition made by the method of any one of Aspect 143-Aspect 149.

Aspect 209. The product of Aspect 208, wherein the product is a savory composition.

Aspect 210. The product of Aspect 209, wherein the savory composition is a food product.

Aspect 211. The product of Aspect 210, wherein the food product is a condiment, a cereal product, a rice product, a pasta product, a tapioca product, a sago product, a baker's product, a biscuit product, a pastry product, a bread product, a yeast product, a mustard product, a vinegar product, a processed food product, a cooked vegetable product, a meat, a meat product, a meat substitute product, an egg product, a dairy product, a cheese product, a dairy substitute product, a soy product, an edible oil, or a fat product.

Aspect 212. The product of Aspect 210, wherein the food product is a snack product such as potato chips, crisps, nuts, tortilla-tostada, pretzels, cheese snacks, corn snacks, potato-snacks, ready-to-eat popcorn, microwaveable popcorn, pork rinds, nuts, crackers, cracker snacks; an aspic product; a cured meat product such as a ham or bacon; a luncheon or breakfast meat product, such as hotdogs, cold cuts, and sausages; a tomato product; a margarine product; a peanut butter product; a soup product such as clear soups, canned soups, cream soups, instant soups, and vegetable or meat broth products; a canned vegetable product; or a pasta sauce product.

Aspect 213. A method for inhibiting bitterness, the method comprising: adding a taste modulator composition to food, beverage, or medicament; wherein the taste modulator composition comprises a first taste modulator component consisting essentially of a first salt having a first cation Mg²⁺ and a first anion; a second taste modulator component consisting essentially of a second salt having a second cation Ca²⁺ and a second anion; and optionally a third taste modulator component consisting essentially of a third salt having a third cation selected from K⁺ and Na⁺ and a third anion; wherein the first taste modulator component is at a concentration of from about 0.1 mM to about 10 mM; wherein the second taste modulator component is at a concentration of from about 0.1 mM to about 10 mM; wherein the third taste modulator component, when present, is at a concentration of from about 0.1 mM to about 25 mM; and wherein the bitterness is inhibited when compared to a baseline product; wherein the baseline product consists essentially of identical components as the product without the taste modulator component; and wherein the bitterness is determined using a sensory panel study.

Aspect 214. The method of Aspect 213, wherein the bitterness is inhibited compared to the baseline product by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, about 101%, about 102%, about 103%, about 104%, about 105%, about 106%, about 107%, about 108%, about 109%, about 110%, about 111%, about 112%, about 113%, about 114%, about 115%, about 116%, about 117%, about 118%, about 119%, about 120%, about 121%, about 122%, about 123%, about 124%, about 125%, about 126%, about 127%, about 128%, about 129%, about 130%, about 131%, about 132%, about 133%, about 134%, about 135%, about 136%, about 137%, about 138%, about 139%, about 140%, about 141%, about 142%, about 143%, about 144%, about 145%, about 146%, about 147%, about 148%, about 149%, about 150%, about 151%, about 152%, about 153%, about 154%, about 155%, about 156%, about 157%, about 158%, about 159%, about 160%, about 161%, about 162%, about 163%, about 164%, about 165%, about 166%, about 167%, about 168%, about 169%, about 170%, about 171%, about 172%, about 173%, about 174%, about 175%, about 176%, about 177%, about 178%, about 179%, about 180%, about 181%, about 182%, about 183%, about 184%, about 185%, about 186%, about 187%, about 188%, about 189%, about 190%, about 191%, about 192%, about 193%, about 194%, about 195%, about 196%, about 197%, about 198%, about 199%, about 200%, or combinations thereof.

Aspect 215. The method of Aspect 214, wherein bitterness is inhibited compared to the baseline product by from about 10% to about 200%.

Aspect 216. The method of Aspect 214, wherein bitterness is inhibited compared to the baseline product by from about 50% to about 200%.

Aspect 217. The method of Aspect 214, wherein bitterness is inhibited compared to the baseline product by from about 100% to about 200%.

Aspect 218. The method of Aspect 214, wherein bitterness is inhibited compared to the baseline product by from about 150% to about 200%.

Aspect 219. The method of Aspect 213, wherein bitterness is inhibited compared to the baseline product by at least 25%.

Aspect 220. The method of Aspect 219, wherein bitterness is inhibited compared to the baseline product by at least 30%.

Aspect 221. The method of Aspect 219, wherein bitterness is inhibited compared to the baseline product by at least 40%.

Aspect 222. The method of Aspect 219, wherein bitterness is inhibited compared to the baseline product by at least 50%.

Aspect 223. The method of Aspect 219, wherein bitterness is inhibited compared to the baseline product by at least 75%.

Aspect 224. The method of Aspect 219, wherein bitterness is inhibited compared to the baseline product by at least 100%.

Aspect 225. The method of Aspect 213, wherein the bitterness of the composition is decreased compared to the baseline composition by about 1.1-fold, about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 2.6-fold, about 2.7-fold, about 2.8-fold, about 2.9-fold, about 3-fold, about 3.1-fold, about 3.2-fold, about 3.3-fold, about 3.4-fold, about 3.5-fold, about 3.6-fold, about 3.7-fold, about 3.8-fold, about 3.9-fold, about 4-fold, about 4.1-fold, about 4.2-fold, about 4.3-fold, about 4.4-fold, about 4.5-fold, about 4.6-fold, about 4.7-fold, about 4.8-fold, about 4.9-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, about 20-fold, about 21-fold, about 22-fold, about 23-fold, about 24-fold, about 25-fold, about 26-fold, about 27-fold, about 28-fold, about 29-fold, about 30-fold, about 31-fold, about 32-fold, about 33-fold, about 34-fold, about 35-fold, about 36-fold, about 37-fold, about 38-fold, about 39-fold, about 40-fold, about 41-fold, about 42-fold, about 43-fold, about 44-fold, about 45-fold, about 46-fold, about 47-fold, about 48-fold, about 49-fold, about 50-fold, about 51-fold, about 52-fold, about 53-fold, about 54-fold, about 55-fold, about 56-fold, about 57-fold, about 58-fold, about 59-fold, about 60-fold, about 61-fold, about 62-fold, about 63-fold, about 64-fold, about 65-fold, about 66-fold, about 67-fold, about 68-fold, about 69-fold, about 70-fold, about 71-fold, about 72-fold, about 73-fold, about 74-fold, about 75-fold, about 76-fold, about 77-fold, about 78-fold, about 79-fold, about 80-fold, about 81-fold, about 82-fold, about 83-fold, about 84-fold, about 85-fold, about 86-fold, about 87-fold, about 88-fold, about 89-fold, about 90-fold, about 91-fold, about 92-fold, about 93-fold, about 94-fold, about 95-fold, about 96-fold, about 97-fold, about 98-fold, about 99-fold, about 100-fold, about 100-fold, about 101-fold, about 102-fold, about 103-fold, about 104-fold, about 105-fold, about 106-fold, about 107-fold, about 108-fold, about 109-fold, about 110-fold, about 111-fold, about 112-fold, about 113-fold, about 114-fold, about 115-fold, about 116-fold, about 117-fold, about 118-fold, about 119-fold, about 120-fold, about 121-fold, about 122-fold, about 123-fold, about 124-fold, about 125-fold, about 126-fold, about 127-fold, about 128-fold, about 129-fold, about 130-fold, about 131-fold, about 132-fold, about 133-fold, about 134-fold, about 135-fold, about 136-fold, about 137-fold, about 138-fold, about 139-fold, about 140-fold, about 141-fold, about 142-fold, about 143-fold, about 144-fold, about 145-fold, about 146-fold, about 147-fold, about 148-fold, about 149-fold, about 150-fold, about 151-fold, about 152-fold, about 153-fold, about 154-fold, about 155-fold, about 156-fold, about 157-fold, about 158-fold, about 159-fold, about 160-fold, about 161-fold, about 162-fold, about 163-fold, about 164-fold, about 165-fold, about 166-fold, about 167-fold, about 168-fold, about 169-fold, about 170-fold, about 171-fold, about 172-fold, about 173-fold, about 174-fold, about 175-fold, about 176-fold, about 177-fold, about 178-fold, about 179-fold, about 180-fold, about 181-fold, about 182-fold, about 183-fold, about 184-fold, about 185-fold, about 186-fold, about 187-fold, about 188-fold, about 189-fold, about 190-fold, about 191-fold, about 192-fold, about 193-fold, about 194-fold, about 195-fold, about 196-fold, about 197-fold, about 198-fold, about 199-fold, about 200-fold or combinations thereof.

Aspect 226. The method of any one of Aspect 213-Aspect 225, wherein the product is a food product.

Aspect 227. The method of Aspect 226, wherein the food product is a condiment, a cereal product, a rice product, a pasta product, a tapioca product, a sago product, a baker's product, a biscuit product, a pastry product, a bread product, a yeast product, a mustard product, a vinegar product, a processed food product, a cooked vegetable product, a meat, a meat product, a meat substitute product, an egg product, a dairy product, a cheese product, a dairy substitute product, a soy product, an edible oil, or a fat product.

Aspect 228. The method of Aspect 226, wherein the food product is a snack product such as potato chips, crisps, nuts, tortilla-tostada, pretzels, cheese snacks, corn snacks, potato-snacks, ready-to-eat popcorn, microwaveable popcorn, pork rinds, nuts, crackers, cracker snacks; an aspic product; a cured meat product such as a ham or bacon; a luncheon or breakfast meat product, such as hotdogs, cold cuts, and sausages; a tomato product; a margarine product; a peanut butter product; a soup product such as clear soups, canned soups, cream soups, instant soups, and vegetable or meat broth products; a canned vegetable product; or a pasta sauce product.

Aspect 229. The method of any one of Aspect 213-Aspect 228, wherein the product is a beverage.

Aspect 230. The method of Aspect 229, wherein the beverage is a juice product; a fruit juice product; a vegetable juice product; a carbonated soft drink product; a beer; a wine; a hot chocolate product; a tea; or a coffee beverage.

Aspect 231. The method of any one of Aspect 213-Aspect 230, wherein the first taste modulator component is at a concentration of from about 1 mM to about 10 mM; and wherein the second taste modulator component is at a concentration of from about 1 mM to about 10 mM.

Aspect 232. The method of Aspect 231, wherein the first taste modulator component is at a concentration of from about 1 mM to about 5 mM; and wherein the second taste modulator component is at a concentration of from about 1 mM to about 5 mM.

Aspect 233. The method of any one of Aspect 213-Aspect 232, wherein the first anion is selected from citrate, chloride, phosphate, carbonate, sulfate, and combinations thereof.

Aspect 234. The method of Aspect 223, wherein the first anion is selected from citrate, sulfate, chloride, and combinations thereof.

Aspect 235. The method of any one of Aspect 213-Aspect 234, wherein the second anion is selected from citrate, chloride, phosphate, carbonate, sulfate, and combinations thereof.

Aspect 236. The composition of Aspect 235, wherein the second anion is selected from citrate, sulfate, chloride, and combinations thereof.

Aspect 237. The method of any one of Aspect 213-Aspect 236, wherein the third taste modulator component is at a concentration of from about 1 mM to about 25 mM.

Aspect 238. The composition of Aspect 237, wherein the third taste modulator component is at a concentration of from about 1 mM to about 15 mM.

Aspect 239. The composition of Aspect 238, wherein the third taste modulator component is at a concentration of from about 5 mM to about 15 mM.

Aspect 240. The method of any one of Aspect 213-Aspect 239, wherein the third anion is selected from citrate, chloride, phosphate, carbonate, sulfate, and combinations thereof.

Aspect 241. The composition of Aspect 240, wherein the third anion is selected from citrate, sulfate, chloride, and combinations thereof.

Aspect 242. The method of Aspect 213-Aspect 241, wherein the concentration of the first taste modulator component is from about 0.1 mM to about 5 mM; wherein the concentration of the second taste modulator component is from about 0.1 mM to about 5 mM; and where the concentration of the third taste modulator component is from about 0.1 mM to about 25 mM.

Aspect 243. The method of Aspect 213-Aspect 242, wherein the pH of the product has a pH from about pH 2.5 to about pH 7.

In various further aspects, the present disclosure relates to the further exemplary aspects provided herein below.

Further Aspect 1. A product comprising: a taste modulator composition comprising of a first taste modulator component comprising a first salt having a first cation Mg²⁺ and a first anion; a second taste modulator component consisting essentially of a second salt having a second cation Ca²⁺ and a second anion; and optionally a third taste modulator component consisting essentially of a third salt having a third cation selected from K⁺ and Na⁺ and a third anion; wherein the first taste modulator component is at a concentration of from about 0.1 mM to about 10 mM; wherein the second taste modulator component is at a concentration of from about 0.1 mM to about 10 mM; wherein the third taste modulator component, when present, is at a concentration of from about 0.1 mM to about 25 mM; wherein the product is a food product, a beverage product, or a medicament; wherein the product is associated with a bitter taste in the absence of the taste modulator composition; wherein the product comprising the taste modulator composition has a bitterness that is lower compared to a baseline product; wherein the baseline product consists essentially of the same components as the product without the taste modulator component; and wherein the bitterness is determined using a sensory panel study.

Further Aspect 2. The product of Further Aspect 1, wherein the first taste modulator component is at a concentration of from about 1 mM to about 10 mM; and wherein the second taste modulator component is at a concentration of from about 1 mM to about 10 mM.

Further Aspect 3. The product of Further Aspect 1, wherein the first taste modulator component is at a concentration of from about 1 mM to about 5 mM; and wherein the second taste modulator component is at a concentration of from about 1 mM to about 5 mM.

Further Aspect 4. The product of Further Aspect 1, wherein the first anion is selected from citrate, chloride, phosphate, carbonate, sulfate, and combinations thereof.

Further Aspect 5. The product of Further Aspect 4, wherein the first anion is selected from citrate, sulfate, chloride, and combinations thereof.

Further Aspect 6. The product of Further Aspect 1, wherein the second anion is selected from citrate, chloride, phosphate, carbonate, sulfate, and combinations thereof.

Further Aspect 7. The product of Further Aspect 6, wherein the second anion is selected from citrate, sulfate, chloride, and combinations thereof.

Further Aspect 8. The product of Further Aspect 1, wherein the third taste modulator component is at a concentration of from about 1 mM to about 25 mM.

Further Aspect 9. The product of Further Aspect 8, wherein the third taste modulator component is at a concentration of from about 1 mM to about 15 mM.

Further Aspect 10. The product of Further Aspect 8, wherein the third taste modulator component is at a concentration of from about 5 mM to about 15.

Further Aspect 11. The product of Further Aspect 1, wherein the third anion is selected from citrate, chloride, phosphate, carbonate, sulfate, and combinations thereof.

Further Aspect 12. The product of Further Aspect 1, wherein the third anion is selected from citrate, sulfate, chloride, and combinations thereof.

Further Aspect 13. The product of Further Aspect 1, wherein the concentration of the first taste modulator component is from about 0.1 mM to about 5 mM; wherein the concentration of the second taste modulator component is from about 0.1 mM to about 5 mM; and where the concentration of the third taste modulator component is from about 0.1 mM to about 25 mM.

Further Aspect 14. The product of Further Aspect 1, wherein the taste modulator composition comprises the first taste modulator component, the second taste modulator component, and optionally the third taste modulator component in an amount sufficient to diminish bitterness by at least 10% compared to the baseline product.

Further Aspect 15. The product of Further Aspect 14, wherein the taste modulator composition comprises the first taste modulator component, the second taste modulator component, and optionally the third taste modulator component in an amount sufficient to diminish bitterness by at least 20% compared to the baseline product.

Further Aspect 16. The product of Further Aspect 14, wherein the taste modulator composition comprises the first taste modulator component, the second taste modulator component, and optionally the third taste modulator component in an amount sufficient to diminish bitterness by at least 30% compared to the baseline product.

Further Aspect 17. The product of Further Aspect 14, wherein the taste modulator composition comprises the first taste modulator component, the second taste modulator component, and optionally the third taste modulator component in an amount sufficient to diminish bitterness by at least 40% compared to the baseline product.

Further Aspect 18. The product of Further Aspect 14, wherein the taste modulator composition comprises the first taste modulator component, the second taste modulator component, and optionally the third taste modulator component in an amount sufficient to diminish bitterness by at least 50% compared to the baseline product.

Further Aspect 19. The product of Further Aspect 14, wherein the taste modulator composition comprises the first taste modulator component, the second taste modulator component, and optionally the third taste modulator component in an amount sufficient to diminish bitterness by at least 60% compared to the baseline product.

Further Aspect 20. The product of Further Aspect 14, wherein the taste modulator composition comprises the first taste modulator component, the second taste modulator component, and optionally the third taste modulator component in an amount sufficient to diminish bitterness by at least 70% compared to the baseline product.

Further Aspect 21. The product of Further Aspect 14, wherein the taste modulator composition comprises the first taste modulator component, the second taste modulator component, and optionally the third taste modulator component in an amount sufficient to diminish bitterness by at least 80% compared to the baseline product.

Further Aspect 22. The product of Further Aspect 14, wherein the taste modulator composition comprises the first taste modulator component, the second taste modulator component, and optionally the third taste modulator component in an amount sufficient to diminish bitterness by at least 90% compared to the baseline product.

Further Aspect 23. The product of Further Aspect 14, wherein the taste modulator composition comprises the first taste modulator component, the second taste modulator component, and optionally the third taste modulator component in an amount sufficient to diminish bitterness by at least 100% compared to the baseline product.

Further Aspect 24. The product of any one of 1-23, wherein the bitterness of the product is lowered by at least 10% compared to the baseline product.

Further Aspect 25. The product of Further Aspect 24, wherein the bitterness the product is lowered by at least 20% compared to the baseline product.

Further Aspect 26. The product of Further Aspect 24, wherein the bitterness of the product is lowered by at least 30% compared to the baseline product.

Further Aspect 27. The product of Further Aspect 24, wherein the bitterness of the product is lowered by at least 40% compared to the baseline product.

Further Aspect 28. The product of Further Aspect 24, wherein the bitterness of the product is lowered by at least 50% compared to the baseline product.

Further Aspect 29. The product of Further Aspect 24, wherein the bitterness of the product is lowered by at least 60% compared to the baseline product.

Further Aspect 30. The product of Further Aspect 24, wherein the bitterness of the product is lowered by at least 70% compared to the baseline product.

Further Aspect 31. The product of Further Aspect 24, wherein the bitterness of the product is lowered by at least 80% compared to the baseline product.

Further Aspect 32. The product of Further Aspect 24, wherein the bitterness of the product is lowered by at least 90% compared to the baseline product.

Further Aspect 33. The product of Further Aspect 24, wherein the bitterness of the product is lowered by at least 100% compared to the baseline product.

Further Aspect 34. The product of any one of 1-33, wherein the product is a beverage product.

Further Aspect 35. The product of Further Aspect 34, wherein the product comprises cranberry juice, grapefruit juice, lime juice, lemon juice, or combinations thereof.

Further Aspect 36. The product of Further Aspect 34, wherein the product comprises cranberry flavor, grapefruit flavor, lime flavor, lemon flavor, or combinations thereof.

Further Aspect 37. The product of Further Aspect 1, wherein the product is a medicament.

Further Aspect 38. The product of Further Aspect 37, wherein the medicament is administered orally.

Further Aspect 39. The product of Further Aspect 38, wherein the medicament is formulated as a pill, powder, granule, elixir, tincture, suspension, syrup, or emulsion.

Further Aspect 40. A method for inhibiting bitterness in a product, the method comprising: adding a taste modulator composition to a product; wherein the taste modulator composition comprises a first taste modulator component consisting essentially of a first salt having a first cation Mg²⁺ and a first anion; a second taste modulator component consisting essentially of a second salt having a second cation Ca²⁺ and a second anion; and optionally a third taste modulator component consisting essentially of a third salt having a third cation selected from K⁺ and Na⁺ and a third anion; wherein the first taste modulator component is at a concentration of from about 0.1 mM to about 10 mM; wherein the second taste modulator component is at a concentration of from about 0.1 mM to about 10 mM; wherein the third taste modulator component, when present, is at a concentration of from about 0.1 mM to about 25 mM; wherein the product is a food product, a beverage product, or a medicament; wherein the product is associated with a bitter taste in the absence of adding the taste modulator composition; wherein the product comprising the taste modulator composition has a bitterness that is lower compared to a baseline product; wherein the baseline product consists essentially of the same components as the product without the taste modulator component; and wherein the bitterness is determined using a sensory panel study.

Further Aspect 41. The method of Further Aspect 40, wherein bitterness is diminished by at least 10% compared to the baseline product.

Further Aspect 42. The method of Further Aspect 41, wherein bitterness is diminished by at least 20% compared to the baseline product.

Further Aspect 43. The method of Further Aspect 41, wherein bitterness is diminished by at least 30% compared to the baseline product.

Further Aspect 44. The method of Further Aspect 41, wherein bitterness is diminished by at least 40% compared to the baseline product.

Further Aspect 45. The method of Further Aspect 41, wherein bitterness is diminished by at least 50% compared to the baseline product.

Further Aspect 46. The method of Further Aspect 41, wherein bitterness is diminished by at least 60% compared to the baseline product.

Further Aspect 47. The method of Further Aspect 41, wherein bitterness is diminished by at least 70% compared to the baseline product.

Further Aspect 48. The method of Further Aspect 41, wherein bitterness is diminished by at least 80% compared to the baseline product.

Further Aspect 49. The method of Further Aspect 41, wherein bitterness is diminished by at least 90% compared to the baseline product.

Further Aspect 50. The method of Further Aspect 41, wherein bitterness is diminished by at least 100% compared to the baseline product.

Further Aspect 51. The method of any one of Further Aspects 40-50, wherein the product is a beverage product.

Further Aspect 52. The method of Further Aspect 51, wherein the product comprises cranberry juice, grapefruit juice, lime juice, lemon juice, or combinations thereof.

Further Aspect 53. The method of Further Aspect 51, wherein the product comprises cranberry flavor, grapefruit flavor, lime flavor, lemon flavor, or combinations thereof.

Further Aspect 54. The method of any one of Further Aspects 40-Further Aspect 53, wherein the product is a medicament.

Further Aspect 55. The method of Further Aspect 54, wherein the medicament is administered orally.

Further Aspect 56. The method of Further Aspect 55, wherein the medicament is formulated as a pill, powder, granule, elixir, tincture, suspension, syrup, or emulsion.

Further Aspect 57. The method of any one of Further Aspects 40-56, wherein the first taste modulator component is at a concentration of from about 1 mM to about 10 mM; and wherein the second taste modulator component is at a concentration of from about 1 mM to about 10 mM.

Further Aspect 58. The method of any one of Further Aspects 40-56, wherein the first taste modulator component is at a concentration of from about 1 mM to about 5 mM; and wherein the second taste modulator component is at a concentration of from about 1 mM to about 5 mM.

Further Aspect 59. The method of any one of Further Aspects 40-56, wherein the first anion is selected from citrate, chloride, phosphate, carbonate, sulfate, and combinations thereof.

Further Aspect 60. The method of Further Aspect 59, wherein the first anion is selected from citrate, sulfate, chloride, and combinations thereof.

Further Aspect 61. The method of any one of Further Aspects 40-60, wherein the second anion is selected from citrate, chloride, phosphate, carbonate, sulfate, and combinations thereof.

Further Aspect 62. The method of Further Aspect 61, wherein the second anion is selected from citrate, sulfate, chloride, and combinations thereof.

Further Aspect 63. The method of any one of Further Aspects 40-62, wherein the third taste modulator component is at a concentration of from about 1 mM to about 25 mM.

Further Aspect 64. The method of Further Aspect 63, wherein the third taste modulator component is at a concentration of from about 1 mM to about 15 mM.

Further Aspect 65. The product of Further Aspect 63, wherein the third taste modulator component is at a concentration of from about 5 mM to about 15.

Further Aspect 66. The method of any one of Further Aspects 40-65, wherein the third anion is selected from citrate, chloride, phosphate, carbonate, sulfate, and combinations thereof.

Further Aspect 67. The method of any one of Further Aspects 40-66, wherein the third anion is selected from citrate, sulfate, chloride, and combinations thereof.

Further Aspect 68. The product of any one of Further Aspects 1-39, wherein taste modulator composition consists essentially of a first taste modulator component consisting essentially of a first salt having a first cation Mg²⁺ and a first anion; a second taste modulator component consisting essentially of a second salt having a second cation Ca²⁺ and a second anion; and optionally a third taste modulator component consisting essentially of a third salt having a third cation selected from K⁺ and Na⁺ and a third anion; wherein the first taste modulator component is at a concentration of from about 0.1 mM to about 10 mM; wherein the second taste modulator component is at a concentration of from about 0.1 mM to about 10 mM; wherein the third taste modulator component, when present, is at a concentration of from about 0.1 mM to about 25 mM.

Further Aspect 69. The method of any one of Further Aspects 40-67, wherein the taste modulator composition consists essentially of a first taste modulator component consisting essentially of a first salt having a first cation Mg²⁺ and a first anion; a second taste modulator component consisting essentially of a second salt having a second cation Ca²⁺ and a second anion; and optionally a third taste modulator component consisting essentially of a third salt having a third cation selected from K⁺ and Na⁺ and a third anion; wherein the first taste modulator component is at a concentration of from about 0.1 mM to about 10 mM; wherein the second taste modulator component is at a concentration of from about 0.1 mM to about 10 mM; wherein the third taste modulator component, when present, is at a concentration of from about 0.1 mM to about 25 mM.

From the foregoing, it will be seen that aspects herein are well adapted to attain all the ends and objects hereinabove set forth together with other advantages which are obvious and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and sub-combinations are of utility and may be employed without reference to other features and sub-combinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, ingredient concentrations are weight/volume (e.g., mg/L) or molar/millimolar, temperature is in ° C. or is at ambient temperature, and pressure is at or near atmospheric.

### Example 1. Effect of a Representative Disclosed Taste Modulation Formulation on Bitterness with Stevia Sweetener Preparations

It is known that some Stevia sweetener preparations are associated with a bitter off-taste sensory sensation, and the level of bitterness can vary with processing method and overall purity of the preparation. This study assessed the effects of a disclosed taste modulation formulation on sensory perceptions, e.g., Sweetness Intensity (SI), Sourness Intensity (Sol), Saltiness Intensity (Sal), Bitterness Intensity (BI), Mouthfeel Intensity (MF), Astringency Intensity (AI), Sweetness Appearance Time (AT), Sweetness Linger (SL) and Sweetness Desensitization (SD) using various commercial Stevia sweetener preparations.

### Experiment 1

Materials: The Stevia sweetener preparations are as described herein. The 3 commercial tabletop sweeteners purchased were as follows:
Commercial Tabletop Stevia Sweetener 1 (TSS 1): The package label indicated this product to be 2.0 g/packet of erythritol and Stevia leaf extract and for 1 packet to be equivalent to 2 teaspoons of sugar. Further the package label indicated the Stevia extract to be rebaudioside D to taste like sugar and have no aftertaste.
Commercial Tabletop Stevia Sweetener 2 (TSS 2): The package label indicated this product to be 1.0 g/packet organic erythritol and organic Stevia extract. Further the package label indicated that one packet is equivalent to 2 teaspoons of sugar.
Commercial Tabletop Stevia Sweetener 3 (TSS 3): The package label indicated this product to be 1.0 g/packet dextrose and Stevia leaf extract. Further the package label indicated each packet to have the sweetness of 2 teaspoons of sugar.

The sensory protocol was generally as described above, but with Sweetness Intensity and Bitterness Intensity as the only descriptors rated; they were rated on 0-15 scales.

The commercial tabletop Stevia sweeteners were evaluated at (a) 1 packet/4 oz (120 mL); and (b) at 2 packets/4 oz (120 mL) distilled water. The results of this evaluation are shown in Table 1 below.

TABLE 1

| | One Packet/4 Oz | | Two Packets/4 Oz | |
|---|---|---|---|---|
| Sweetener | Sweetness | Bitterness | Sweetness | Bitterness |
| TSS 1 | 6 | 0 | 8 | 0 |
| TSS 2 | 6 | 2 | 8 | 5 |
| TSS 3 | 4 | 0 | 8 | 3 |

The data obtained shows that certain commercial Stevia preparations are associated with a concentration-dependent bitterness sensory component.

From the foregoing analysis it was determined that the TSS 2 sweetener was a suitable Stevia sweetener preparation to test for bitterness inhibition using a disclosed taste modulation composition. The following two samples were then prepared:
(a) 2 packets of TSS 2 tabletop sweetener in 100 mL distilled water; and
(b) 2 packets of TSS 2 tabletop sweetener in 100 mL distilled water with KCl @ 10 mM, MgCl₂.6H₂O @ 3 mM and CaCl₂ @ 3 mM.

The two samples were then evaluated in duplicate with the following sensory protocol described above and briefly summarized here:
(1) taste the TSS 2/10 mM KCl/3 mM MgCl₂/3 mM CaCl₂ sample swirling gently in the mouth for 15 sec and expectorate;
(2) rinse with 15 mL water and expectorate @ 30 sec;
(3) rate intensities of Sweetness, Saltiness, Bitterness, Mouthfeel, Astringency on a 0-15 scales and Sweetness Appearance Time [R=Rapid (0.0), D=Delay (2.5), SD=Significant Delay (5.0)];
(4) observe the Sweetness Rebound after the water rinse and rate Sweetness Linger @ 2 min 30 sec on a 0-5 scale;
(5) rinse with 15 mL water, observe Sweetness Desensitization and rate [N=None (0.0), S=Slight (2.5), M=Medium (5.0)]; and
(6) repeat Steps 1-5 w/the TSS 2 Sample after a 15 min break.

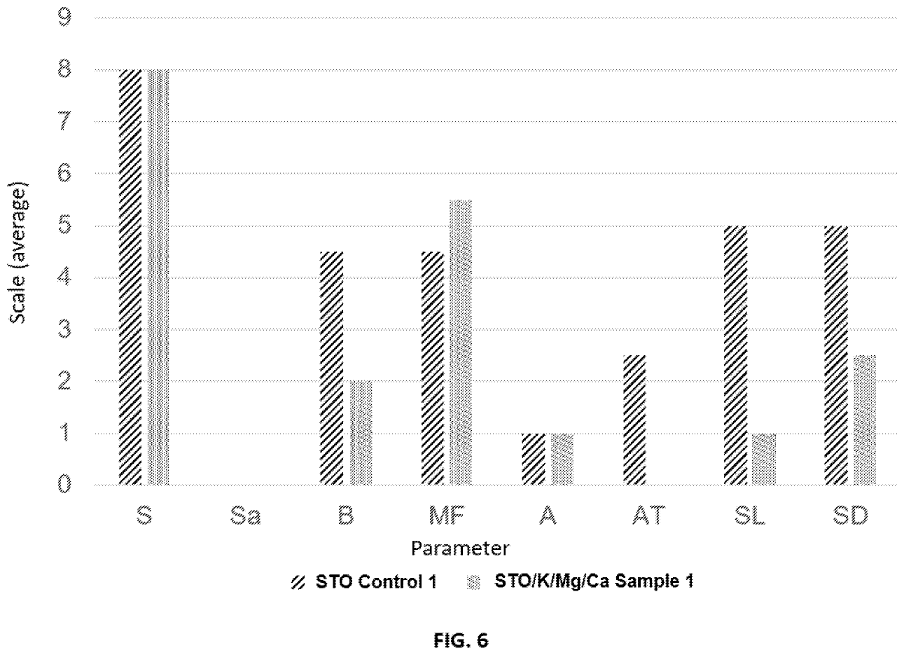

The results obtained in the foregoing study are shown below in Table 2 and FIG. 6.

TABLE 2

| Sample | S | So | Sa | B | MF | A | AT | SL | SD |
|---|---|---|---|---|---|---|---|---|---|
| TSS 2 w/KCl/MgCl₂/ | 8 | 0 | 0 | 2 | 5.5 | 1 | 0 | 1 | 2.5 |
| CaCl₂ | | | | | | | | | |
| TSS 2 | 8 | 0 | 0 | 4.5 | 4.5 | 1 | 2.5 | 5 | 5 |

The data show that a representative taste modulator composition, i.e., 10 mM KCl/3 mM MgCl₂/3 mM CaCl₂, markedly reduces the bitter off taste of the TSS 2 tabletop sweetener. At the same time, this taste modulator composition also increases the Mouthfeel of the TSS 2 tabletop sweetener, accelerates its sweetness Appearance Time, reduces its Sweetness Linger and also reduces the Sweetness Desensitization observed for this commercial product. It is noteworthy that the TSS 2 Stevia sweetener preparation exhibits significant Mouthfeel even in the absence of the taste modulator. Without wishing to be bound by a particular theory, it is believed that significant baseline Mouthfeel observed with the TSS 2 Stevia sweetener preparation is a consequence of the high level of erythritol in the product.

### Experiment 2

Materials: a commercial Stevia sweetener preparation having a weak bitter off-taste was tested. The commercial Stevia sweetener preparation, referred to herein as “STEV”, comprises not less than 95 wt % steviol glycosides, of which, not less than 50 wt % is Rebaudioside A, and the balance comprises a mixture of compounds comprising a steviol backbone conjugated to any number or combination of the principal sugar moieties (glucose, rhamnose, xylose, fructose, arabinose, galactose and deoxyglucose) in any of the orientations occurring in the leaves of Stevia rebaudiana Bertoni” (JECFA 2017 definition). All other materials were as described above for Example 1.

In this experiment, the effectiveness of a representative disclosed taste modulator composition comprising KCl/MgCl₂.6H₂O/CaCl₂ was evaluated for inhibition of bitterness observed in STEV, both in distilled water and in citric acid buffer (CAB), prepared by addition of 10% KOH to 1.50 g citric acid monohydrate in 1 L distilled water until a pH of 3.2 was reached. The following samples were prepared:

(1) 500 mg/L STEV in water;

(2) 500 mg/L STEV and 5 mM KCl/3 mM MgCl₂.6H₂O/3 mM CaCl₂ in water;

(3) 500 mg/L STEV in CAB; and

(4) 500 mg/L STEV and 5 mM KCl/3 mM MgCl₂.6H₂O/3 mM CaCl₂ in CAB.

The foregoing four samples were then evaluated with the sensory protocol as described immediately above in Experiment 1 of Example 1.

The results of this analysis are as shown in Table 3 below.

TABLE 3

| Sample | S | So | Sa | B | MF | A | AT | SL | SD |
|---|---|---|---|---|---|---|---|---|---|
| 500 mg/L STEV in Water | 8 | 0 | 0 | 2 | 0 | 1 | 2.5 | 5 | 5 |
| 500 mg/L STEV/5 mM | 8 | 0 | 0 | 1 | 5 | 1 | 0 | 3 | 5 |
| KCl/3 mM MgCl₂/3 mM | | | | | | | | | |
| CaCl₂ in Water | | | | | | | | | |
| 500 mg/L STEV in CAB | 7 | 2 | 0 | 1 | 0 | 2 | 0 | 3 | 2.5 |
| 500 mg/L STEV/5 mM | 8 | 2 | 0 | 0 | 4 | 2 | 0 | 1 | 0 |
| KCl/3 mM MgCl₂/3 mM | | | | | | | | | |
| CaCl₂ in CAB | | | | | | | | | |

The data show a marked improvement in mouthfeel for this Stevia sweetener preparation in both water and citric acid buffer, as well as clear reduction in bitterness off-taste.

### Example 2. Effect of Representative Disclosed Taste Modulation Formulation on Bitterness of Amino Acids

Experiment 1. A 30 mM solution of L-Phenylalanine (L-Phe) in pH 3.2 Citric Acid Buffer (CAB) was prepared and serial dilutions from 30 mM to 1.88 mM were prepared with CAB. The serial dilutions were then tasted in ascending order and the Bitter Taste Threshold in CAB was estimated to be ca. 3 mM. The 7.5 mM L-Phe solution was estimated to have a bitterness intensity of 5 on the 0-15 intensity scale described in Example 1. A 7.5 mM solution of L-Phe in CAB was then prepared in combination with 10 mM KCl/3 mM MgCl₂.6H₂O/3 mM CaCl₂. This L-Phe solution exhibited no detectable bitterness.

Experiment 2. A 30 mM solution of L-Tryptophan (L-Trp) in pH 3.2 Citric Acid Buffer (CAB) was prepared and serial dilutions from 30 mM to 1.88 mM were prepared with CAB. The serial dilutions were then tasted in ascending order and the Bitter Taste Threshold in CAB was estimated to be ca. 3 mM. The 7.5 mM L-Trp solution was estimated to have a bitterness intensity of 5 on the 0-15 intensity scale described in Example 1. The bitterness of L-Trp in CAB was noticeably slow in onset and quite lingering. The 7.5 mM solution of L-Trp in CAB was then prepared in combination with 10 mM KCl/3 mM MgCl₂.6H₂O/3 mM CaCl₂. This L-Trp solution exhibited a marked reduction in bitterness estimated to be 2 on the 0-15 intensity scale described in Example 1.

### Example 3. Effect of Representative Disclosed Taste Modulation Formulation on Bitterness of Commercial Juice, Protein Hydrolysate and Other Beverages

Experiment 1. The effect of representative disclosed taste modulation formulations on the bitterness of tonic water was tested. Quinine salts are used as the bittering agents in commercial tonic waters. A commercial tonic water was evaluated after addition of the 5 mM KCl/3 mM MgCl₂.6H₂O/3 mM CaCl₂ taste modulator and found to be very significantly less bitter than the beverage lacking the taste modulator. Similarly, the same commercial tonic water was evaluated in the presence of a 10 mM KCl/3 mM MgSO₄.7H₂O/Ca(Lactate)₂.5H₂O taste modulator and also observed to be very significantly less bitter than the beverage lacking the taste modulator. Thus, it is clear that both the 5 mM KCl/3 mM MgCl₂.6H₂O/3 mM CaCl₂ taste modulator and the 10 mM KCl/3 mM MgSO₄.7H₂O/Ca(Lactate)₂.5H₂O taste modulator are very effective in inhibition of the bitterness of quinine.

Experiment 2. The effect of representative disclosed taste modulation formulations on the bitterness of grapefruit juice beverages was tested. Naringin is the dominant bitter principle in grapefruit juice. A commercial grapefruit juice was evaluated in the presence of a 10 mM KCl/3 mM MgSO₄.7H₂O/Ca(Lactate)₂.5H₂O taste modulator. It was observed to be significantly less bitter than the grapefruit juice beverage lacking the taste modulator. Similarly, a commercial sugar free red grapefruit juice sweetened with sucralose and acesulfame-K was evaluated in the presence of a 10 mM KCl/3 mM MgSO₄.7H₂O/Ca(Lactate)₂.5H₂O taste modulator. It also was observed to be significantly less bitter than the sugar-free red grapefruit juice beverage lacking the taste modulator. Thus, it is clear that the 10 mM KCl/3 mM MgSO₄.7H₂O/Ca(Lactate)₂.5H₂O taste modulator is very effective in inhibition of the bitterness of naringin.

Experiment 3. The effect of representative disclosed taste modulation formulations on the bitterness of a cranberry-lime juice beverage was tested. Cranberry juice is known to contain several natural products with bitter and astringent off tastes including flavonoids, proanthocyanidins, anthocyanins, phenolic acids and ellagitannins. A commercial sugar-free cranberry-lime beverage sweetened with sucralose and acesulfame-K was evaluated in the presence of a 10 mM KCl/3 mM MgSO₄.7H₂O/Ca(Lactate)₂.5H₂O taste modulator. It was observed to be very significantly less bitter than the sugar-free cranberry-lime juice beverage lacking the taste modulator. Thus, it is clear that the 10 mM KCl/3 mM MgSO₄.7H₂O/Ca(Lactate)₂.5H₂O taste modulator is effective in inhibition of the bitter off tastes of some or all of the bitter natural products in cranberry and lime juices.

Experiment 4. The effect of representative disclosed taste modulation formulations on the bitterness of protein hydrolysate beverages was tested. Protein hydrolysates are commonly known to contain many peptides which exhibit bitter off tastes. A commercial vanilla-créme flavored milk protein hydrolysate beverage sweetened with sucralose and acesulfame-K was evaluated after addition of the 5 mM KCl/3 mM MgCl₂.6H₂O/3 mM CaCl₂ taste modulator and found to be very significantly less bitter than the beverage lacking the taste modulator. The taste modulator fortified beverage was also observed to be less astringent and to have a much improved quality of taste. Similarly, organic pea protein hydrolysate rebaudioside A/erythritol sweetened beverages were prepared with and without the 5 mM KCl/3 mM MgCl₂.6H₂O/3 mM CaCl₂ taste modulator. Again, the formulation containing the taste modulator exhibited significantly less bitter off taste. Thus, it is clear that the 5 mM KCl/3 mM MgCl₂.6H₂O/3 mM CaCl₂ taste modulator is very effective in inhibition of the bitter off tastes of peptides formed in protein hydrolysis.

Experiment 5. The effect of representative disclosed taste modulation formulations on the bitterness of energy beverages was tested. Caffeine is known to be a bittering compound in many beverage applications. A commercial energy beverage containing 114 mg of caffeine per 12 oz beverage and sweetened with aspartame and acesulfame-K was evaluated after addition of the 5 mM KCl/3 mM MgCl₂.6H₂O/3 mM CaCl₂ taste modulator and found to be very significantly less bitter than the beverage lacking the taste modulator. The taste modulator fortified beverage was also observed to be less astringent and to have a much improved quality of taste. Very similar results in bitter off taste reduction were made on addition of 10 mM KCl/3 mM MgSO₄.7H₂O/3 mM Ca(Lactate)₂.5H₂O to the commercial energy beverage. Thus, it is clear that the 5 mM KCl/3 mM MgCl₂.6H₂O/3 mM CaCl₂ taste modulator as well as the 10 mM KCl/3 mM MgSO₄.7H₂O/3 mM Ca(Lactate)₂.5H₂O taste modulator are very effective in inhibition of the bitter off taste of caffeine.

Experiment 6. The effect of representative disclosed taste modulation formulations on the bitterness of beer and ale beverages was tested. Beers and ales are well known to have a strong bitter taste derived from isohumulones and other components of hops. A commercial India Pale Ale was evaluated after addition of the 5 mM KCl/3 mM MgCl₂.6H₂O/3 mM CaCl₂ taste modulator and found to be markedly less bitter than the beverage lacking the taste modulator and to be quite pleasant in taste. In addition, the bitterness in the beverage containing the taste modulator did not build in intensity over time as was the case for the commercial ale beverage.

It should be emphasized that the above-described aspects, including the tables herein, of the present disclosure are merely possible examples of implementations set forth for a clear understanding of the principles of the disclosure. Many variations and modifications may be made to the above-described aspect(s) without departing substantially from the spirit and principles of the disclosure. All such modifications and variations are intended to be included herein within the scope of this disclosure and protected by the following claims.

What is claimed:

1. A method for inhibiting bitterness in a product, the method comprising:
adding a taste modulator composition to a product;
wherein the taste modulator composition consists essentially of
a first taste modulator component consisting essentially of a first salt having a first cation Mg²⁺ and a first anion;
a second taste modulator component consisting essentially of a second salt having a second cation Ca²⁺ and a second anion; and optionally
a third taste modulator component consisting essentially of a third salt having a third cation selected from K⁺ and Na⁺ and a third anion;
wherein the first taste modulator component is at a concentration of from about 0.1 mM to about 10 mM;
wherein the second taste modulator component is at a concentration of from about 0.1 mM to about 10 mM;
wherein the third taste modulator component, when present, is at a concentration of from about 0.1 mM to about 25 mM;
wherein the product is a food product, a beverage product, or a medicament;
wherein the product is associated with a bitter taste in the absence of adding the taste modulator composition;
wherein the product comprising the taste modulator composition has a bitterness that is lower compared to a baseline product;
wherein the baseline product consists essentially of the same components as the product without the taste modulator component; and
wherein the bitterness is determined using a sensory panel study.

2. The method of claim 1, wherein bitterness is diminished by at least 10% compared to the baseline product.

3. The method of claim 2, wherein bitterness is diminished by at least 20% compared to the baseline product.

4. The method of claim 2, wherein bitterness is diminished by at least 30% compared to the baseline product.

5. The method of claim 2, wherein bitterness is diminished by at least 40% compared to the baseline product.

6. The method of claim 2, wherein bitterness is diminished by at least 50% compared to the baseline product.

7. The method of claim 2, wherein bitterness is diminished by at least 60% compared to the baseline product.

8. The method of claim 2, wherein bitterness is diminished by at least 70% compared to the baseline product.

9. The method of claim 2, wherein bitterness is diminished by at least 80% compared to the baseline product.

10. The method of claim 2, wherein bitterness is diminished by at least 90% compared to the baseline product.

11. The method of claim 2, wherein bitterness is diminished by at least 100% compared to the baseline product.

12. The method claim 1, wherein the product is a beverage product.

13. The method of claim 12, wherein the product comprises cranberry juice, grapefruit juice, lime juice, lemon juice, or combinations thereof.

14. The method of claim 12, wherein the product comprises cranberry flavor, grapefruit flavor, lime flavor, lemon flavor, or combinations thereof.

15. The method claim 1, wherein the product is a medicament.

16. The method of claim 15, wherein the medicament is administered orally.

17. The method of claim 16, wherein the medicament is formulated as a pill, powder, granule, elixir, tincture, suspension, syrup, or emulsion.

18. The method claim 1, wherein the first taste modulator component is at a concentration of from about 1 mM to about 10 mM; and wherein the second taste modulator component is at a concentration of from about 1 mM to about 10 mM.

19. The method claim 1, wherein the first taste modulator component is at a concentration of from about 1 mM to about 5 mM; and wherein the second taste modulator component is at a concentration of from about 1 mM to about 5 mM.

20. The method claim 1, wherein the first anion is selected from citrate, chloride, phosphate, carbonate, sulfate, and combinations thereof.

21. The method of claim 20, wherein the first anion is selected from citrate, sulfate, chloride, and combinations thereof.

22. The method claim 1, wherein the second anion is selected from citrate, chloride, phosphate, carbonate, sulfate, and combinations thereof.

23. The method of claim 22, wherein the second anion is selected from citrate, sulfate, chloride, and combinations thereof.

24. The method claim 1, wherein the third taste modulator component is at a concentration of from about 1 mM to about 25 mM.

25. The method of claim 24, wherein the third taste modulator component is at a concentration of from about 1 mM to about 15 mM.

26. The product of claim 24, wherein the third taste modulator component is at a concentration of from about 5 mM to about 15.

27. The method claim 1, wherein the third anion is selected from citrate, chloride, phosphate, carbonate, sulfate, and combinations thereof.

28. The method claim 1, wherein the third anion is selected from citrate, sulfate, chloride, and combinations thereof.

29. A product comprising:
a taste modulator composition consisting essentially of
a first taste modulator component consisting essentially of a first salt having a first cation Mg²⁺ and a first anion;
a second taste modulator component consisting essentially of a second salt having a second cation Ca²⁺ and a second anion; and optionally
a third taste modulator component consisting essentially of a third salt having a third cation selected from K⁺ and Na⁺ and a third anion;
wherein the first taste modulator component is at a concentration of from about 0.1 mM to about 10 mM;
wherein the second taste modulator component is at a concentration of from about 0.1 mM to about 10 mM;
wherein the third taste modulator component, when present, is at a concentration of from about 0.1 mM to about 25 mM;
wherein the product is a food product, a beverage product, or a medicament;
wherein the product is associated with a bitter taste in the absence of the taste modulator composition;
wherein the product comprising the taste modulator composition has a bitterness that is lower compared to a baseline product;
wherein the baseline product consists essentially of the same components as the product without the taste modulator component; and
wherein the bitterness is determined using a sensory panel study.

30. The product of claim 29, wherein the first taste modulator component is at a concentration of from about 1 mM to about 10 mM; and wherein the second taste modulator component is at a concentration of from about 1 mM to about 10 mM.
